SEQUENCE LISTING (1 sequence)

SEQ ID NO: 1 - DNA, 3315 bp, Homo sapiens
atgtccttcgagggagccaggctcagcatgaggagccgcagaaatggtactatgggcagcacccggaccctgtactccagtgtatctcggagcacagacgtgtcctacagtgacagtgatttggtgaattttattcaggcaaattttaaaaaacgagaatgtgtcttctttaccagagactccaaggccatggagaacatatgcaagtgtggttatgcccagagccagcacatcgaaggcacccagatcaaccaaaatgagaagtggaactacaaaaaacataccaaggagtttccaacagacgccttcggggacattcagtttgagactctggggaagaaaggcaagtacttacgcttgtcctgtgacaccgactctgaaactctctacgaactgctgacccagcactggcacctcaaaacacccaacctggtcatttcagtgacgggtggagccaaaaactttgctttgaagccacgcatgcgcaagatcttcagcaggctgatttacatcgcacagtctaaaggtgcgtggattctcactggaggcactcactacggcctgatgaagtacataggcgaggtggtgagagacaacaccatcagcaggaactcagaagagaacatcgtggccattggcatcgcagcatggggcatggtctccaacagggacaccctcatcaggagctgtgatgatgagggacatttttcagctcaatacatcatggatgactttaccagagaccctctatacatcctggacaacaaccatacccacctgctgcttgtggacaacggttgtcatggacaccccacagtggaagccaagctccggaatcagctggaaaagtacatctctgagcgcaccagtcaagattccaactatggtggtaagatccccatcgtgtgttttgcccaaggaggtggaagagagactctaaaagccatcaacacctctgtcaaaagcaagatcccttgtgtggtggtggaaggctcggggcagattgctgatgtgatcgccagcctggtggaggtggaggatgttttaacctcttccatggtcaaagagaagctggtacgctttttaccacgcactgtgtcccggctgcctgaagaggaaattgagagctggatcaaatggctcaaagaaattcttgagagttctcacctactcacagtaattaagatggaagaggctggagatgagattgtgagcaacgccatttcctatgcgctgtacaaagccttcagcactaatgagcaagacaaggacaactggaatggacagctgaagcttctgctggagtggaaccagttggaccttgccagtgatgagatcttcaccaatgatcgccgctgggagtctgccgaccttcaggaggtcatgttcacggctctcataaaggacagacccaagtttgtccgcctctttctggagaatggcctgaatctgcagaagtttctcaccaatgaagtcctcacagagctcttctccacccacttcagcaccctagtgtaccggaatctgcagatcgccaagaactcctacaatgacgcactcctcacctttgtctggaagttggtggcaaacttccgtcgaagcttctggaaagaggacagaagcagcagggaggacttggatgtggaactccatgatgcatctctcaccacccggcacccgctgcaagctctcttcatctgggccattcttcagaacaagaaggaactctccaaggtcatttgggagcagaccaaaggctgtactctggcagccttgggggccagcaagcttctgaagaccctggccaaagttaagaatgatatcaacgctgctggggaatcggaggaactggccaatgaatatgagacccgagcagtggagttgttcaccgagtgttacagcaatgatgaagacttggcagaacagctactggtctactcctgcgaagcctggggtgggagcaactgtctggagctggcagtggaggctacagatcagcatttcatcgctcagcctggggtccagaatttcctttctaagcaatggtatggagagatttcccgagacacgaagaactggaagattatcctgtgtctattcatcatccccttagtgggctgtggcctcgtatcatttaggaagaaacccattgacaagcacaagaagctgctgtggtactatgtggccttcttcacgtcgcccttcgtggtcttctcctggaacgtggtcttctacatcgccttcctcctgctgtttgcctatgtgctgctcatggacttccactcagtgccacacacccccgagctgatcctctacgccctggtcttcgtcctcttctgtgatgaagtgaggcagtggtacatgaacggagtgaattatttcaccgacctatggaacgttatggacaccctgggactcttctacttcatagcgggtattgtattccggctccactcttctaataaaagctcgttgtactctgggcgcgtcattttctgtctggattacattatattcacgctaaggctcatccacattttcaccgtcagcaggaacttgggacccaagattataatgctgcagcggatgctgatcgacgttttcttcttcctgttcctctttgctgtgtggatggtggcctttggcgtggccagacaggggatcctaaggcaaaatgaacagcgctggagatggatcttccgctctgtcatctatgagccctacctggccatgtttggccaggttcccagtgacgtggatagtaccacatatgacttctcccactgtaccttctcgggaaatgagtccaagccactgtgtgtggagctggatgagcacaacctgccccgcttccctgagtggatcaccattccgctggtgtgcatctacatgctctccaccaatatccttctggtcaacctcctggtcgccatgtttggctacacggtaggcattgtacaggagaacaacgaccaggtctggaaattccagcggtacttcctggtgcaggagtactgcaaccgcctaaacatccccttccccttcgttgtcttcgcttatttctacatggtggtgaagaagtgtttcaaatgctgctgtaaagagaagaatatggagtctaatgcctgctgtttcagaaatgaggacaatgagactttggcgtgggagggtgtcatgaaggagaattaccttgtcaagatcaacacgaaagccaacgacaactcagaggagatgaggcatcggtttagacaactggactcaaagcttaacgacctcaaaagtcttctgaaagagattgctaataacatcaagtaa

(19) United States
(12) Patent Application Publication
(10) Pub. No.: US 2018/0312463 A1
(43) Pub. Date: Nov. 1, 2018

(54) Synthesis of Cyclohexane Carboxamide Derivatives Useful as Sensates in Consumer Products

(71) Applicant: The Procter & Gamble Company, Cincinnati, OH (US)

(72) Inventors: Kenneth Edward YELM, Hamilton, OH (US); John August WOS, Mason, OH (US); Gregory Mark BUNKE, Lawrenceburg, IN (US); Heath FREDERICK, Cincinnati, OH (US); John Christian HAUGHT, West Chester, OH (US); Steven Hamilton HOKE, West Chester, OH (US); Koti Tatachar SREEKRISHNA, Cincinnati, OH (US); Yakang LIN, Liberty Township, OH (US)

(21) Appl. No.: 16/030,382

(22) Filed: Jul. 9, 2018

Related U.S. Application Data
(63) Continuation of application No. 15/331,783, filed on Oct. 21, 2016.
(60) Provisional application No. 62/245,192, filed on Oct. 22, 2015.

(51) Int. Cl. C07C 237/10; C07C 269/06; C07C 231/02; C07D 213/81; C07C 319/20; A61K 8/41; A61Q 11/00; C07C 233/62; C07C 271/20; C07C 271/22; C07C 237/22; C07C 233/78; C07C 321/14

(52) U.S. Cl. CPC C07C 237/10; C07C 321/14; C07C 269/06; C07C 231/02; C07B 2200/07; C07D 213/81; C07C 319/20; C07C 2601/14; A61K 8/41; A61Q 11/00; C07C 233/62; C07C 271/20; C07C 271/22; C07C 237/22; C07C 233/78; A61K 2800/244

(57) ABSTRACT

Synthesis methods to produce a series of carboxamides built off of an (S)-2-amino acid backbone or an (R)-2-amino acid backbone, depending upon the desired diastereomer of the end product.

### FIELD OF THE INVENTION

The present invention relates to the synthesis of cyclohexane-based derivatives useful as sensates. In particular the present synthetic route can be used to prepare various isomers of cyclohexane-based carboxamide coolants.

### BACKGROUND OF THE INVENTION

Oral care products, such as dentifrice and mouthwash, are routinely used by consumers as part of their oral care hygiene regimens. It is well known that oral care products can provide both therapeutic and cosmetic hygiene benefits to consumers. Therapeutic benefits include caries prevention, which is typically delivered through the use of various fluoride salts; gingivitis prevention, by the use of an antimicrobial agent such as stannous fluoride, triclosan, essential oils; or hypersensitivity control through the use of ingredients such as strontium chloride or potassium nitrate. Cosmetic benefits provided by oral care products include the control of plaque and calculus formation, removal and prevention of tooth stain, tooth whitening, breath freshening, and overall improvements in mouth feel impression, which can be broadly characterized as mouth feel aesthetics. Calculus and plaque along with behavioral and environmental factors lead to formation of dental stains, significantly affecting the aesthetic appearance of teeth. Behavioral and environmental factors that contribute to teeth staining propensity include regular use of coffee, tea, cola or tobacco products, and also the use of certain oral products containing ingredients that promote staining, such as cationic antimicrobials and metal salts.

Thus daily oral care at home requires products with multiple ingredients working by different mechanisms to provide the complete range of therapeutic and aesthetic benefits, including anticaries, antimicrobial, antigingivitis, antiplaque, anticalculus and anti-erosion, as well as antiodor, mouth refreshment, stain removal, stain control and tooth whitening. In order for daily use oral care products, such as dentifrice and rinses to provide complete oral care it is often necessary to combine actives and additives, many of which have the disadvantage of causing negative aesthetics during use, in particular unpleasant taste, unpleasant sensations and stain promotion. The unpleasant taste and mouth sensations have been described as having one or more of bitter, metallic, astringent, salty, numbing, stinging, burning, or prickling, and even irritating aspects. Typical ingredients for oral care use that are associated with these aesthetic negatives include antimicrobial agents such as cetyl pyridinium chloride, chlorhexidine, stannous and zinc salts, tooth bleaching agents such as peroxides; antitartar agents such as pyrophosphate, tripolyphosphate and hexametaphosphate; and excipients such as baking soda and surfactants. To mitigate the aesthetic negatives from these ingredients, oral care products are typically formulated with flavoring agents, sweeteners and coolants to taste as good as possible and provide a pleasant experience. In particular, it is desirable for oral care products to provide a refreshing cooling sensation during and after use. In addition to mitigation of negative sensations, sensate molecules are formulated into oral care compositions to convey a signal of efficacy. Such signals of efficacy include cooling, tingling, numbing, warming, sweetness, and rheological sensations such as phase change and fizzing or bubbling.

A large number of coolant compounds of natural or synthetic origin have been described. The most well-known compound is menthol, particularly 1-menthol, which is found naturally in peppermint oil, notably of Mentha arvensis L and Mentha viridis L. Of the menthol isomers, the 1-isomer occurs most widely in nature and is typically what is referred by the name menthol having coolant properties. L-menthol has the characteristic peppermint odor, has a clean fresh taste and exerts a cooling sensation when applied to the skin and mucosal surfaces. Other isomers of menthol (neomenthol, isomenthol and neoisomenthol) have somewhat similar, but not identical odor and taste, i.e., some having disagreeable notes described as earthy, camphor, musty. The principal difference among the isomers is in their cooling potency. L-menthol provides the most potent cooling, i.e., having the lowest cooling threshold of about 800 ppb, i.e., the concentration where the cooling effect could be clearly recognized. At this level, there is no cooling effect for the other isomers. For example, d-neomenthol is reported to have a cooling threshold of about 25,000 ppb and 1-neomenthol about 3,000 ppb. (R. Emberger and R. Hopp, “Synthesis and Sensory Characterization of Menthol Enantiomers and Their Derivatives for the Use in Nature Identical Peppermint Oils,” Specialty Chemicals (1987), 7(3), 193-201). This study demonstrated the outstanding sensory properties of 1-menthol in terms of cooling and freshness and the influence of stereochemistry on the activity of these molecules. Among synthetic coolants, many are derivatives of or are structurally related to menthol, i.e., containing the cyclohexane moiety, and derivatized with functional groups including carboxamide, ketal, ester, ether and alcohol. Examples include the p-menthanecarboxamide compounds, such as N-ethyl-p-menthan-3-carboxamide, known commercially as “WS-3”, and others in the series, such as WS-5 (N-ethoxycarbonylmethyl-p-menthan-3-carboxamide), WS-12 [N-(4-methoxyphenyl)-ρ-menthan-3-carboxamidel and WS-14 (N-tert-butyl-ρ-menthan-3-carboxamide). Examples of menthane carboxy esters include WS-4 and WS-30. An example of a synthetic carboxamide coolant that is structurally unrelated to menthol is N,2,3-trimethyl-2-isopropylbutanamide, known as “WS-23”. Additional examples of synthetic coolants include alcohol derivatives such as 3-(1-menthoxy)-propane-1,2-diol known as TK-10, isopulegol (under the tradename Coolact P) and ρ-menthane-3,8-diol (under the tradename Coolact 38D); menthone glycerol acetal known as MGA; menthyl esters such as menthyl acetate, menthyl acetoacetate, menthyl lactate known as Frescolat* supplied by Haarmann and Reimer, and monomenthyl succinate under the tradename Physcool from V. Mane. TK-10 is described in U.S. Pat. No. 4,459,425. Other alcohol and ether derivatives of menthol are described e.g., in GB 1,315,626 and in U.S. Pat. Nos. 4,029,759; 5,608,119; and 6,956,139. WS-3 and other carboxamide cooling agents are described for example in U.S. Pat. Nos. 4,136,163; 4,150,052; 4,153,679; 4,157,384; 4,178,459 and 4,230,688. Additional N-substituted ρ-menthane carboxamides are described in WO 2005/049553A1 including N-(4-cyanomethylphenyl)-ρ-menthanecarboxamide, N-(4-sulfamoylphenyl)-ρ-menthanecarboxamide, N-(4-cyanophenyl)-ρ-menthanecarboxamide, N-(4-acetylphenyl)-ρ-menthanecarboxamide, N-(4-hydroxymethylphenyl)-ρ-menthanecarboxamide and N-(3-hydroxy-4-methoxyphenyl)-ρ-menthanecarboxamide. Other N-substituted p-menthane carboxamides include amino acid derivatives such as those disclosed in WO 2006/103401 and in U.S. Pat. Nos. 4,136,163; 4,178,459 and 7,189,760 such as N-((5-methyl-2-(1-methylethyl)cyclohexyl)carbonyl)glycine ethyl ester and N((5-methyl-2-(1-methylethyl)cyclohexyl)carbonyealanine ethyl ester. Menthyl esters including those of amino acids such as glycine and alanine are disclosed e.g., in EP 310 299 and in U.S. Pat. Nos. 3,111,127; 3,917,613; 3,991,178; 5,703,123; 5,725,865; 5,843,466; 6,365,215; 6,451,844; and 6,884,903. Ketal derivatives are described, e.g., in U.S. Pat. Nos. 5,266,592; 5,977,166 and 5,451,404. Additional agents that are structurally unrelated to menthol but have been reported to have a similar physiological cooling effect include alpha-keto enamine derivatives described in U.S. Pat. No. 6,592,884 including 3-methyl-2-(1-pyrrolidinyl)-2-cyclopenten-1-one (3-MPC), 5-methyl-2-(1-pyrrolidinyl)-2-cyclopenten-1-one (5-MPC), and 2,5-dimethyl-4-(1-pyrrolidinyl)-3(2H)-furanone (DMPF); icilin (also known as AG-3-5, chemical name 1-[2-hydroxyphenyl]-4-[2-nitrophenyl]-1,2,3,6-tetrahydropyrimidine-2-one) described in Wei et al., J. Pharm. Pharmacol. (1983), 35:110-112. Reviews on the coolant activity of menthol and synthetic coolants include H. R. Watson, et al. J. Soc. Cosmet. Chem. (1978), 29, 185-200 and R. Eccles, J. Pharm. Pharmacol., (1994), 46, 618-630.

Molecules with chiral centers can drive different biological responses depending upon the spatial orientation of specific moieties on those molecules. The biological responses tend to differ where these molecules interact with a receptor. In the flavor and fragrance realm, a well-known example of such chiral diversity is Carvone. The R-(−) enantiomers of Carvone connote a spearmint taste and scent, where the S-(+) enantiomer has a taste and smell like caraway seeds. Limonene is another molecule where the spatial orientation of the chiral center affects its scent. For example, the R-(+) isomer of limonene has a citrus scent, where the S-(−) isomer smells like turpentine. For synthetic molecules, the ability to control the stereochemistry during the synthesis steps, gives the ability to select for the finished molecule with the desired sensorial properties. The object of this invention is a method of synthesis to control the stereochemistry of amino acid substituted cyclohexane carboxamides.

The present invention provides one or more methods for synthesizing carboxamides having a desired stereochemistry and provide a cooling sensation.

### SUMMARY OF THE INVENTION

A method is provided for preparing an N-menthanecarboxamide derivative of the following formula (I) comprising:

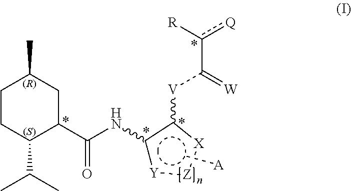

R₁ is independently selected from H, alkyl, aryl, amino alkyl, alkoxy, alkoxy carbonyl, alkyl carbonyl, aryl carbonyl, heteroaryl carbonyl
Q=H₂, O, OR₁, N(R₁)₂
V═—(CH₂)ₘN(R₁)₂, O, OR₁; m=0 to 6
W═H₂, O
X, Y=independently selected from H, alkyl, aryl, arylalkyl, cycloalkyl, naphthyl for n=0
X, Y=aliphatic CH₂ or aromatic CH for n≥1 and Z is selected from aliphatic CH₂, aromatic CH, or heteroatom
A=lower alkoxy, lower alkylthio, aryl, subsitituted aryl or fused aryl and stereochemistry is variable at the positions marked*, and pharmaceutically acceptable salts thereof;
A) a coupling reaction between an activated derivative of the p-menthane-3-carboxylic acid of Formula (II):

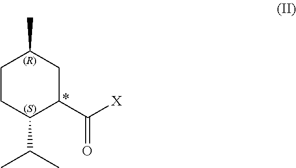

Wherein X=a suitable leaving group; and a primary amine of the general formula (III):

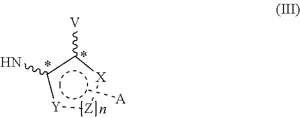

to produce a compound of the general formula (IV):

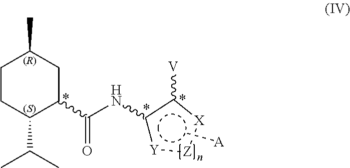

A method is provided for preparing an N-menthanecarboxamide derivative of the following formula (VI) comprising:

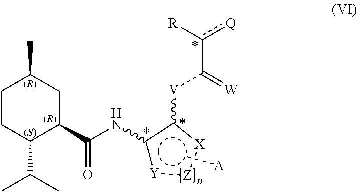

R₁ is independently selected from H, alkyl, aryl, amino alkyl, alkoxy, alkoxy carbonyl, alkyl carbonyl, aryl carbonyl, heteroaryl carbonyl
Q=H₂, O, OR₁, N(R₁)₂
V═—(CH₂)ₘN(R₁)₂, O, OR₁; m=0 to 6
W═H₂, O
X, Y=independently selected from H, alkyl, aryl, arylalkyl, cycloalkyl, naphthyl for n=0
X, Y=aliphatic CH₂ or aromatic CH for n≥1 and Z is selected from aliphatic CH₂, aromatic CH, or heteroatom
A=lower alkoxy, lower alkylthio, aryl, subsitituted aryl or fused aryl and stereochemistry is variable at the positions marked*, and pharmaceutically acceptable salts thereof;
A) a coupling reaction between an activated derivative of the p-menthane-3-carboxylic acid of Formula (VII):

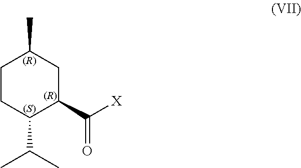

wherein X=a suitable leaving group; and a primary amine of the general formula (VIII):

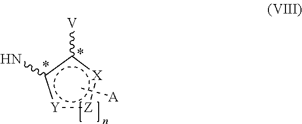

to produce a compound of the general formula (IX):

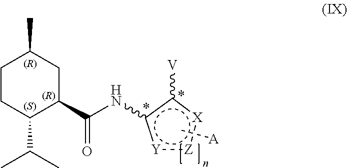

### DETAILED DESCRIPTION OF THE INVENTION

The present invention involves synthesis methods to produce a series of carboxamides built off of an (S)-amino acid backbone or an (R)-2-amino acid backbone, depending upon the desired diastereomer of the end product. Where the amino acid can be in the D or L form and may be natural or unnatural. Examples of amino acids that can be substituted on this backbone include either (D)-alanine, (L)-alanine, or glycine. These molecules have low EC50 values on TRPM8 and drive a neural stimulated cooling response.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. unless otherwise specified.

As used herein, the word “or” when used as a connector of two or more elements is meant to include the elements individually and in combination; for example X or Y, means X or Y or both.

As used herein, the articles “a” and “an” are understood to mean one or more of the material that is claimed or described, for example, “an oral care composition” or “a bleaching agent.”

By “personal care composition” is meant a product, which in the ordinary course of usage is applied to or contacted with a body surface to provide a beneficial effect. Body surface includes skin, for example dermal or mucosal; body surface also includes structures associated with the body surface for example hair, teeth, or nails. Examples of personal care compositions include a product applied to a human body for improving appearance, cleansing, and odor control or general aesthetics. Non-limiting examples of personal care compositions include oral care compositions, such as, dentifrice, mouth rinse, mousse, foam, mouth spray, lozenge, chewable tablet, chewing gum, tooth whitening strips, floss and floss coatings, breath freshening dissolvable strips, denture care product, denture adhesive product; after shave gels and creams, pre-shave preparations, shaving gels, creams, or foams, moisturizers and lotions; cough and cold compositions, gels, gel caps, liquids, and throat sprays; leave-on skin lotions and creams, shampoos, body washes, body rubs, such as Vicks Vaporub; hair conditioners, hair dyeing and bleaching compositions, mousses, shower gels, bar soaps, antiperspirants, deodorants, depilatories, lipsticks, foundations, mascara, sunless tanners and sunscreen lotions; feminine care compositions, such as lotions and lotion compositions directed towards absorbent articles; baby care compositions directed towards absorbent or disposable articles; and oral cleaning compositions for animals, such as dogs and cats.

The term “dentifrice”, as used herein, includes tooth or subgingival -paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

The term “dispenser”, as used herein, means any pump, tube, or container suitable for dispensing compositions such as dentifrices.

The term “teeth”, as used herein, refers to natural teeth as well as artificial teeth or dental prosthesis.

The term “orally acceptable carrier or excipients” includes safe and effective materials and conventional additives used in oral care compositions including but not limited to fluoride ion sources, anti-calculus or anti-tartar agents, buffers, abrasives such as silica, alkali metal bicarbonate salts, thickening materials, humectants, water, surfactants, titanium dioxide, flavorants, sweetening agents, xylitol, coloring agents, and mixtures thereof.

Herein, the terms “tartar” and “calculus” are used interchangeably and refer to mineralized dental plaque biofilms.

The present invention is also directed towards “oral health compositions” as used herein refers to compositions in a form that is deliverable to a mammal in need via the oral cavity, mouth, throat, nasal passage or combinations thereof. Nonlimiting examples include liquid compositions, cough syrups, respiratory preparations, beverage, supplemental water, pills, soft gels, tablets, capsules, gel compositions, foam compositions, saline wash and combinations thereof. Liquid compositions, gel compositions can be in a form that is directly deliverable to the mouth and throat. These compositions or preparations can be delivered by a delivery device selected from droppers, pump, sprayers, liquid dropper, saline wash delivered via nasal passageway, cup, bottle, liquid filled gel, liquid filled gummy, center filled gum, chews, films, center filled lozenge, gum filled lozenge, pressurized sprayers, atomizers, air inhalation devices, liquid filled compressed tablet, liquid filled gelatin capsule, liquid filled capsule, squeezable sachets, power shots, and other packaging and equipment, and combinations thereof. The sprayer, atomizer, and air inhalation devices can be associated with a battery or electric power source.

The present invention is also directed towards a respiratory preparation. In one embodiment the respiratory preparation comprises a film forming agent and a thickening agent. The preparation provides on demand relief. The preparation can work to physically coat the mouth and throat creating a soothing barrier over the epithelial cells that line the throat layer. The preparation can additionally, reduce inflammation and relieve minor pain associated with a cough or sore throat.

The present invention is also directed to lotion compositions and to absorbent articles, particularly disposable absorbent articles, having a lotion treatment composition applied thereon. Disposable absorbent articles can be baby diapers or feminine hygiene articles, including incontinence devices and catamenial products, such as tampons, sanitary napkins, pantiliners, interlabial products, and the like.

An absorbent article may comprise any known or otherwise effective topsheet, such as one which is compliant, soft feeling, and non-irritating to the body of the wearer. Suitable topsheet materials include a liquid pervious material that is oriented towards and contacts the body of the wearer, thereby permitting body discharges to rapidly penetrate through the topsheet without allowing fluid to flow back through the topsheet to the skin of the wearer. The topsheet, while capable of allowing rapid transfer of fluid through it, also provides for the transfer or migration of the lotion composition onto an external or internal portion of a body of the wearer. A suitable topsheet can be made of various materials, such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof.

A lotion composition may comprise at least one rheology structurant, which typically is a solid. The lotion composition can further comprise other optional ingredients, like surface energy modifiers. In certain embodiments, a lotion composition may comprise a rheology structurant, such as a microcrystalline wax, alkyl dimethicone, ethylene glycol dibehenate, ethylene glycol distearate, glycerol tribehenate, glycerol tristearate, and ethylene bisoleamide.

In preparing a lotioned catamenial device according to the present invention, the lotion composition can be applied to the outer surface of the absorbent article, such as, for example, the outer surface of the topsheet. Any of a variety of application methods that distribute lubricious materials having a molten or liquid consistency can be used. Suitable methods include but are not limited to spraying, printing (e.g., flexographic printing), coating (e.g., gravure coating), extrusion, dipping, or combinations of these application techniques, e.g., spraying the lotion composition on a rotating surface, such as a calender roll, that then transfers the composition to the outer surface of the sanitary napkin topsheet.

The following paragraphs describe one or more methods for producing carboxamide structures.

Unlike traditional carboxamide structures, the carboxamides of the present invention were built off of a (S)-2-phenyl glycine backbone or an (R)-2-phenyl glycine backbone, depending upon the desired diastereomer at position 2. It was important to control the stereochemistry at position 2, in order for the end product to be a substantially pure diastereomer and not a mixture of diastereomers. Once the 2-phenylglycine methyl ester was made, the spatial orientation of the amine at position 2 would be locked and carried through the reaction process into the end product. From the 2-phenylglycine methyl ester, the menthyl carboxamide was subsequently converted to the specified coolant product.

General Description for Synthesis of Carboxamide Derivatives (Scheme 1)

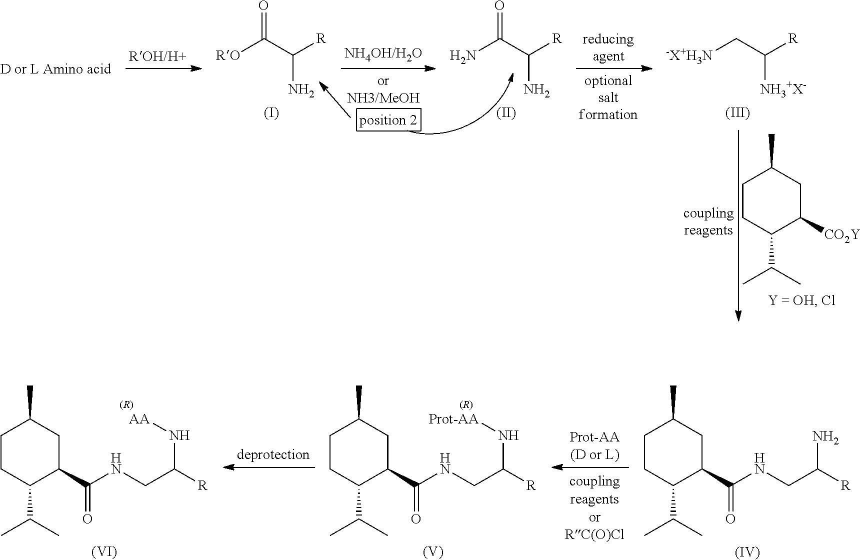

In general, the described carboxamide analogs can be synthesized by the route described in Scheme 1. In a general description, the D or L amino acid is converted to an ester (I) by known esterification methods common in the art (ie: J. Med. Chem., 2015, 3144). The ester can then be coverted to an amide via amidation using one of several forms of ammonia to provide the amide (II) , as is described in the art (ie: Cao, Sheldon; et al PCT 2012171506). The following amides (II) can be prepared, based on the amidation transformation described in Scheme 1, and in TABLE 1.

TABLE 1
R-group conversion to amide (II)

| # | R-group | Product (II) |
|---|---|---|
| 1 | (S)-alanine | (S)-2-aminopropanamide |
| 2 | (S)-phenylalanine | (S)-2-amino-3-phenylpropanamide |
| 3 | (S)-histidine | (S)-2-amino-3-(1H-imidazol-4-yl) propanamide |
| 4 | (S)-isoleucine | (2S,3S)-2-amino-3-methylpentanamide |
| 5 | (S)-leucine | (S)-2-amino-4-methylpentanamide |
| 6 | (S)-serine | (S)-2-amino-3-hydroxypropanamide |
| 7 | (S)-threonine | (2S,3R)-2-amino-3-hydroxybutanamide |
| 8 | (S)-valine | (S)-2-amino-3-methylbutanamide |
| 9 | (S)-tryptophan | (S)-2-amino-3-(1H-indol-3-yl)propanamide |
| 10 | (S)-tyrosine | (S)-2-amino-3-(4-hydroxyphenyl)propanamide |
| 11 | (S)-naphthyl alanine | (S)-2-amino-3-(naphthalene-2-yl)propanamide |
| 12 | (S)-phenyl glycine | (S)-2-amino-2-phenylacetamide |

The amide (II) can be reduced to the diamine (III) using a reducing agent, such as lithium aluminum hydride (LAH) or other reducing agent as disclosed in the art (ie: US Pub. No. 2014/206673). The resulting diamine (III) can be optionally converted to a salt for isolation and purification purposes, or can be used as the diamine directly in the coupling step. The following diamines (III) can be prepared, based on the reduction step described in Scheme 1 (II)-(III), and in TABLE 2.

TABLE 2
R-group conversion to diamine (III)

| # | R-group | Product (III) |
|---|---|---|
| 13 | (S)-alanine | (S)-propane-1,2-diamine dihydrochloride |
| 14 | (S)-phenylalanine | (S)-3-phenylpropane-1,2-diamine dihydrochloride |
| 15 | (S)-histidine | (S)-3-(1H-imidazol-4-yl)propane-1,2-diamine |
| | | dihydrochloride |
| 16 | (S)-isoleucine | (2S,3S)-3-methylpentane-1,2-diamine dihydrochloride |
| 17 | (S)-leucine | (S)-4-methylpentane-1,2-diamine dihydrochloride |
| 18 | (S)-serine | (S)-2,3-diaminopropan-1-ol dihydrochloride |
| 19 | (S)-threonine | (2S,3R)-diaminobutan-2-ol dihydrochloride |
| 20 | (S)-valine | (S)-3-methylbutane-1,2-diamine |
| 21 | (S)-tryptophan | (S)-3-(1H-indol-3-yl)propane-1,2-diamine dihydrochloride |
| 22 | (S)-tyrosine | (S)-4-(2,3-diaminopropyl)phenol dihydrochloride |
| 23 | (S)-naphthyl alanine | (S)-3-(naphthalene-2-yl)propane-1,2-diamine |
| | | dihydrochloride |
| 24 | (S)-phenyl glycine | (S)-1-phenylethane-1,2-diamine dihydrochloride salt |

The diamine (III) can then be coupled to the appropriately functionalized (1R,2S,5R)-N-((S)-2-amino-2-phenylethyl)-2-isopropyl-5-methylcyclohexane derivative to provide the cyclohexane carboxamide derivative (IV) using conditions and reagents such as those disclosed in the art (ie: U.S. Pat. No. 9,181,226). The following carboxamides (IV) can be prepared, based on the coupling step (III)-(IV) in Scheme 1, and in TABLE 3.

TABLE 3
Carboxamide (IV)

| # | R-group | Product (IV) |
|---|---|---|
| 25 | (S)-alanine | (1R,2S,5R)-N((S)-2-aminopropyl)-2-isopropyl-5- |
| | | methylcyclohexane-1-carboxamide |
| 26 | (S)-phenylalanine | (1R,2S,5R)-N((S)-2-amino-3-phenylpropyl)-2-isopropyl-5- |
| | | methylcylohexane-1-carboxamide |
| 27 | (S)-histidine | (1R,2S,5R)-N((S)-2-amino-3-(1H-imidazol-4-yl)propyl)-2- |
| | | isopropyl-5-methylcyclohexane-1-carboxamide |
| 28 | (S)-isoleucine | (1R,2S,5R)-N((S)-2-amino-3-methylpentyl)-2-isopropyl-5- |
| | | methylcyclohexane-1-carboxamide |
| 29 | (S)-leucine | (1R,2S,5R)-N((S)-2-amino-4-methylpentyl)-2-isopropyl-5- |
| | | methylcyclohexane-1-carboxamide |
| 30 | (S)-serine | (1R,2S,5R)-N((R)-2-amino-3-hydroxypropyl)-2-isoproyl-5- |
| | | methylcyclohexane-1-carboxamide |
| 31 | (S)-threonine | (1R,2S,5R)-N((R)-2-amino-3-hydroxybutyl)-2-isopropyl-5- |
| | | methylcyclohexane-1-carboxamide |
| 32 | (S)-valine | (1R,2S,5R)-N((S)-2-amino-3-methylbutyl)-2-isopropyl-5- |
| | | methylcyclohexane-1-carboxamide |
| 33 | (S)-tryptophan | (1R,2S,5R)-N((S)-2-amino-3-(1H-indol-3-yl)propyl)-2- |
| | | isopropyl-5-methylcyclohexane-1-carboxamide |
| 34 | (S)-tyrosine | (1R,2S,5R)-N((S)-2-amino-3-(4-hydroxyphenyl)propyl-2- |
| | | isopropyl-5-methylcyclohexane-1-carboxamide |
| 35 | (S)-naphthyl alanine | (1R,2S,5R)-N((S)-2-amino-3-(naphthalene-2-yl)propyl)-2- |
| | | isopropyl-5-methylcylohexane-1-carboxamide |
| 36 | (S)-phenyl glycine | (1R,2S,5R)-N-((S)-2-amino-2-phenylethyl)-2-isopropyl-5- |
| | | methylcyclohexane-1-carboxamide |

The carboxamide (IV) can subsequently be capped via acylation or alkylation to provide a variety of N-substituted carboxamides (V) which can be further manipulated via deprotection or tested for TRPV activity as independent chemical entities. For example if the capping group is Prot-AA equivqlent to (D)-Boc Ala-OH, then the following N-capped carboxamides would be available as described in TABLE 4, steps (IV-V) in Scheme 1.

TABLE 4
Carboxamide (V)

| # | R-group | Product (D) Boc-Ala (V) |
|---|---|---|
| 37 | (S)-alanine | tert-butyl ((R)-1-(((S)-1-((1R,2S,5R)-2-isopropyl-5-methylcyclohexane-1- |
| | | carboxamido)propan-2-yl)amino)-1-oxopropan- 2-yl)carbamate |
| 38 | (S)-phenylalanine | tert-butyl ((R)-1-(((S)-1-((1R,2S,5R)-2-isopropyl-5-methylcyclohexane-1- |
| | | carboxamido)-3-phenylpropan-2-yl)amino)-1- oxopropan-2-yl)carbamate |
| 39 | (S)-histidine | tert-butyl ((R)-1-(((S)-1-(1H-imidazol-4-yl)-3-((1R,2S,5R)-2-isopropyl-5- |
| | | methylcyclohexane-1-carboxamido)propan-2- yl)amino)-1-oxopropan-2-yl)carbamate |
| 40 | (S)-isoleucine | tert-butyl ((2R)-1-(((2S)-1-((1R,2S,5R)-2-isopropyl-5-methylcyclohexane-1- |
| | | carboxamido)-3-methylpentan-2-yl)amino)-1- oxopropan-2-yl)carbamate |
| 41 | (S)-leucine | tert-butyl ((R)-1-(((S)-1-((1R,2S,5R)-2-isopropyl-5-methylcyclohexane-1- |
| | | carboxamido)-4-methyl-pentan-2-yl)amino)-1- oxopropan-2-yl)carbamate |
| 42 | (S)-serine | tert-butyl ((R)-1-(((R)-1-hydroxy-3-((1R,2S,5R)-2-isopropyl-5-methylcyclohexane- |
| | | 1-carboxamido)propan-2-yl)amino)-1- oxopropan-2-yl)carbamate |
| 43 | (S)-threonine | tert-butyl ((2S)-1-(((3R)-3-hydroxy-1-((1R,2S,5R)-2-isopropyl-5-methylcyclohexane- |
| | | 1-carboxamido)butan-2-yl)amino)-1- oxopropan-2-yl)carbamate |
| 44 | (S)-valine | tert-butyl ((R)-1-(((S)-1-((1R,2S,5R)-2-isopropyl-5-methylcyclohexane-1- |
| | | carboxamido)-3-methyl-butan-2-yl)amino)-1- oxopropan-2-yl)carbamate |
| 45 | (S)-tryptophan | tert-butyl ((R)-1-(((S)-1-(1H-indol-3-yl)-3-((1R,2S,5R)-2-isopropyl- |
| | | 5-methylcyclohexane-1-carboxamido)propan-2-yl) amino)-1-oxopropan-2-yl)carbamate |
| 46 | (S)-tyrosine | tert-butyl ((R)-1-(((S)-1-(4-hydroxyphenyl)-3-((1R,2S,5R)-2-isopropyl- |
| | | 5-methylcyclohexane-1-carboxamido)propan-2-yl) amino)-1-oxopropan-2-yl)carbamate |
| 47 | (S)-naphthyl | tert-butyl ((R)-1-(((S)-1-((1R,2S,5R)-2-isopropyl-5-methylcyclohexane- |
| | alanine | 1-carboxamido)-3-(naphthalen-2-yl)propan-2-yl) amino)-1-oxopropan-2-yl)carbamate |
| 48 | (S)-phenyl | tert-butyl ((R)-1-(((S)-2-((1R,2S,5R)-2-isopropyl-5-methylcyclohexane-1- |
| | glycine | carboxamido)-1-phenylethyl)amino)-1-oxopropan- 2-yl)carbamate |

In a final step in Scheme 1, the N-capping group can be optionally de-protected to provide the carboxamides (VI) as final products. As an example, using (D) Boc-Ala-OH as the N-capping group in the pentultimate intermediate and removing the Boc protecting group, the carboxamides (VI) would be accessible as illustrated in TABLE 5 and in (V-VI) in Scheme 1.

TABLE 5
Carboxamide (VI)

| # | R-group | Product (VI) from (D) Ala-OH N-capping group |
|---|---|---|
| 49 | (S)-alanine | (1R,2S,5R)-N-((S)-2-((R)-2-aminopropanamido) |
| | | propyl)-2-isopropyl-5-methylcyclohexane-1-carboxamide |
| 50 | (S)-phenylalanine | (1R,2S,5R)-N-((S)-2-((R)-2-aminopropanamido)-3-phenylpropyl)-2- |
| | | isopropyl-5-methylcyclo-hexane-1-carboxamide |
| 51 | (S)-histidine | (1R,2S,5R)-N-((S)-2-((R)-2-aminopropanamido)-3-(1H-imidazol-4-yl) |
| | | propyl)-2-isopropyl-5-methylcyclohexane-1-carboxamide |
| 52 | (S)-isoleucine | (1R,2S,5R)-N-((2S)-2-((R)-2-aminopropanamido)-3-methylpentyl)-2- |
| | | isopropyl-5-methylcyclohexane-1-carboxamide |
| 53 | (S)-leucine | (1R,2S,5R)-N-((S)-2-((R)-2-aminopropanamido)-4-methylpentyl)-2- |
| | | isopropyl-5-methylcyclohexane-1-carboxamide |
| 54 | (S)-serine | (1R,2S,5R)-N-((R)-2-((R)-2-aminopropanamido)-3- |
| | | hydroxypropyl)-2-isopropyl-5-methylcyclohexane-1-carboxamide |
| 55 | (S)-threonine | (1R,2S,5R)-N-((3R)-2-((S)-2-aminopropanamido)-3- |
| | | hydroxybutyl)-2-isopropyl-5-methylcyclohexane-1-carboxamide |
| 56 | (S)-valine | (1R,2S,5R)-N-((S)-2-((R)-2-aminopropanamido)-3- |
| | | methylbutyl)-2-isopropyl-5-methylcyclohexane-1-carboxamide |
| 57 | (S)-tryptophan | (1R,2S,5R)-N-((S)-2-((R)-2-aminopropanamido)-3- |
| | | (1H-indol-3-yl)propyl)-2-isopropyl-5-methyl-cyclohexane-1- |
| | | carboxamide |
| 58 | (S)-tyrosine | (1R,2S,5R)-N-((S)-2-((R)-2-aminopropanamido)-3-(4-hydroxyphenyl)propyl)-2- |
| | | isopropyl-5-methyl-cyclohexane-1-carboxamide |
| 59 | (S)-naphthyl | (1R,2S,5R)-N-((S)-2-((R)-2-aminopropanamido-3-(naphthalene-2-yl)propyl)-2- |
| | alanine | isoprpyl-5-methyl-cyclohexane-1-carboxamide |
| 60 | (S)-phenyl | (1R,2S,5R)-N-((S)-2-((R)-2-aminopropanamido)-2-phenylethyl)-2-isopropyl-5- |
| | glycine | methylcyclohexane-1-carboxamide |

General Description for Synthesis of Carboxamide Derivatives (Scheme 2)

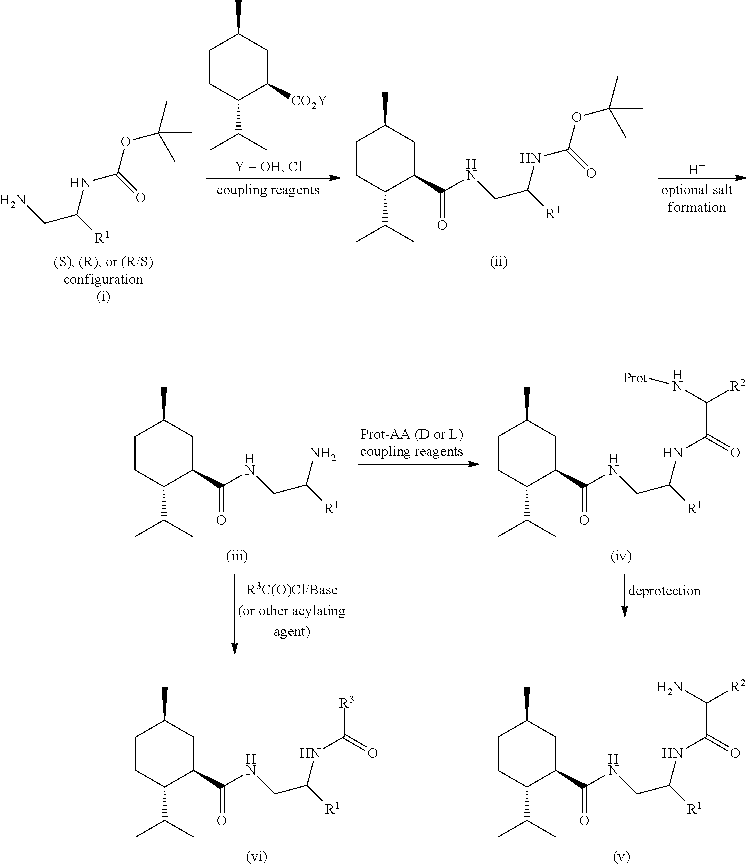

R¹, R², and R³ can be chosen from but are not limited to H, methyl, ethyl, linear or branched C₃-C₁₈ alkyl, heteroatom-substituted alkyl, phenyl, napthyl, other aryl, heteroaryl, benzyl, and other alkylaryl or alkyl heteroaryl groups. R³ can also include substituted alkoxy or amino groups.

In general, carboxamide analogs described can be synthesized by the route descibed in Scheme 2. In a general description the monoprotected (R)-, (S)-, or racemic diamines (i), are available via known art (e.g., US Pub. No. 2014/94462; A European Journal; vol. 12; nb. 26; (2006); p. 6910-6929; Angewandte Chemie—International Edition; vol. 45; nb. 1; (2006); p. 117-120; Journal of Medicinal Chemistry; vol. 37; nb. 12; (1994); p. 1810-1822; Heterocycles; vol. 69; nb. 1; (2006); p. 179-192; Journal of the American Chemical Society; vol. 126; nb. 11; (2004); p. 3418-3419; Journal of Medicinal Chemistry; vol. 48; nb. 13; (2005); p. 4237-4246; Journal of Medicinal Chemistry; vol. 56; nb. 20; (2013); p. 8049-8065).

The monoprotected diamine (i); (from Scheme 2) can then be coupled to the appropriately functionalized (1R,2S,5R)-2-isopropyl-5-methylcyclohexane-1-carboxylic acid derivative to provide the cyclohexane carboxamide derivative (ii) using conditions and reagents such as those disclosed in the art (e.g.: U.S. Pat. No. 9,181,226).

The carboxamide (iii) can subsequently be capped via acylation with activated carboxylic acids (acid chlorides, anhydrides, etc.) to provide N-substituted carboxamide (vi). The carboxamide (iii) can also be capped via acylation with protected amino acids to provide a variety of N-substituted carboxamides (iv) which can be further manipulated via deprotection or tested for TRPV activity as independent chemical entities. Upon deprotection of these materials (iv) the carboxamides (v) are produced.

### EXAMPLES

The following non-limiting EXAMPLES represent molecules synthesized using one or more methods of the present invention. All EXAMPLES were run at room temperature (RT), standard pressure and atmosphere, unless otherwise noted. The water used in the EXAMPLES was deionized water, unless otherwise noted.

### Example 1

### (S)-1-phenylethane-1,2-diamine dihydrochloride salt (B) or (S)-tert-butyl-2-amino-1-phenylethylcarbamate:

### SYNTHESIS of (S)-1-phenylethane-1,2-diamine

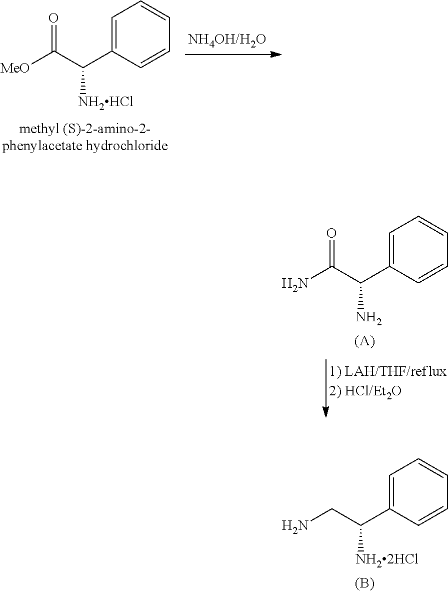

Option A: Synthesis of (S)-1-phenylethane-1,2-diamine dihydrochloride salt (B) Step 1: (S)-2-amino-2-phenylacetamide (A):

Into a 1000 mL 24/40 joint single neck round bottomed flask equipped with a stir bar under nitrogen sparge at room temperature (RT) was added a solution of 50 grams methyl (S)-2-amino-2-phenylacetate hydrochloride (CAS# 15028-39-4, Sigma-Aldrich Corp., St. Louis, Mo.) in water (25 mL). Aqueous NH₄OH (28-30%, 400 mL) was then added dropwise over approximately 20 minutes. The reaction was stirred five days at room temperature under N2 atmosphere, and then concentrated under vacuum (˜5-10 mm Hg) on a rotovap (Buchi Rotovapor R-124, BÜCHI Labortechnik AG, Switzerland). The residue was dissolved in water (250 mL), and extracted with CH₂Cl₂ (5×500 mL) using a separatory funnel. The organic layers were combined into a 4L Erlenmeyerflask, then dried (anhydrous Na₂SO4), filtered to remove drying agent and concentrated via rotovap under vacuum (5-10 mm Hg) to provide a white solid (11.2 grams); MS (ESI): 150.

Step 2:

(S)-1-phenylethane-1,2-diamine dihydrochloride salt (B):

In a 1000 mL 24/40 joint three-neck round-bottom flask equipped with a reflux condenser, a stir bar for stirring, and a nitrogen inlet port, was added (S)-2-amino-2-phenylacetamide (A) (8 g, 0.0533 mol). The reaction was sparged with N₂, then 500 mL anhydrous tetrahydrofuran (THF) was added via cannula. The reaction was stirred 10 minutes to dissolve the acetamide, then solid lithium aluminum hydride (6 g, 95% powder, 0.150 mol) was added portion wise in 6×1 g portions with rapid stirring over approximately 30 minutes. The reaction was then refluxed using a heating mantle until the starting material was consumed (4-6 hrs) as determined by thin layer chromatography (15% MeOH/CH₂Cl₂ as eluent). The reaction was then cooled to room temperature (RT) by removal of the heating mantle and waiting for one hr. The reaction was placed in an ice-water bath for 30 minutes, as stirring was continued; and 10% aqueous NaOH solution (50 mL) was added via additional funnel slowly over 1 hr to quench excess lithium aluminum hydride. After the reaction was quenched, as determined by the ceasing of gas evolution, Filtrol 150 clay (20 g) and Celite 545 (20 g) were added portion wise using a spatula and the mixture was stirred using a stir bar at RT for 2 hr. The mixture was then vacuum filtered (5-10 mm Hg) using a Buchner funnel through a pad of Celite 545 and the pad was washed thoroughly with THF (4×250 mL, 1L total volume). The filtrate was collected in a 2L filter flask and concentrated on a rotovap (Buchi Rotovapor R-124, 5-10 mm Hg) to provide a yellow oil. The oil was dissolved in THF (150 mL) and 2 M HCl/Et₂O (150 mL) was added drop wise with an addition funnel over 15 minutes. The reaction was stirred using a stir bar overnight and the product was filtered off under vacuum (5-10 mm Hg) using a Buchner funnel to provide (S)-1-phenylethane-1,2-diamine dihydrochloride salt (B) as a white solid. 7.3 g; MS: (ESI) 136.

Option B: A Synthesis of (S)-tert-butyl-2-amino-1-phenylethylcarbamate

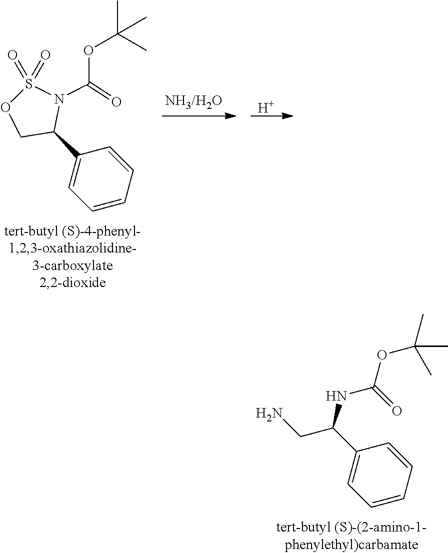

Concentrated ammonium hydroxide (4.0 mL) was added to a 25 mL round bottom flask containing 0.081 g of tert-butyl (S)-4-phenyl-1,2,3-oxathiazolidine-3-carboxylate 2,2-dioxide and a magnetic stir bar. The slurry was stirred (partially dissolved) for 3 h. After cooling the reaction mixture in an ice bath, 6 mL of concentrated hydrochloric acid was added over 10 min. More white precipitate formed and after stirring 2.5 h in the ice bath the mixture was made basic by the addition of 4 mL of 50% aqueous sodium hydroxide solution. The mixture was approximately pH 11 (pH test strips) with less suspended solids present. An additional 10 mL of water was added and the mixture was extracted twice with 15 mL of CH₂Cl₂. The combined organic layers were extracted with 20 mL of water, then with 10 mL of saturated aqueous sodium chloride, and dried over sodium sulfate. The sodium sulfate was removed by filtration and the solvent was evaporated by rotary evaporation under vacuum to give the product. MS (ESI): m/z 237 (40%, MH+for product), 181 (100%,-56,-isobutylene).

### Example 2

### Synthesis of (R)-tert-butyl-2-amino-1-phenylethylcarbamate

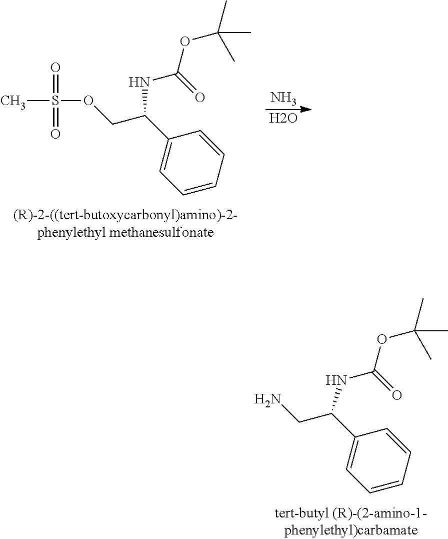

A 20 mL stainless steel Parr pressure reactor containing a magnetic stir bar was charged with 0.111 g (0.352 mmol) of (R)-2-((tert-butoxycarbonyl)amino)-2-phenylethyl methanesulfonate and 5 mL of concentrated ammonium hydroxide to make a white suspension of solid. The system was pressurized with 150 psi of argon and vented five times before leaving under 100 psi of argon. The reactor was placed in a 65° C. oil bath and stirred. The reactor attained a pressure of 119 psi. After 15.25 h the reactor was cooled and vented. The contained solution was nearly homogenous. The reactor contents were transferred to a 100 mL conical flask and rinsed with 15 mL of water. The mixture was concentrated on a rotary evaporator under vacuum (up to 40° C., 45 min) to provide a white solid which was taken up in 1 mL of methanol plus 15 mL of methylene chloride. This solution was extracted successively with 15 mL 0.1 M NaOH, 15 mL water, 15 mL brine, and the organic phase was dried over sodium sulfate. After filtering off the sodium sulfate the solution was concentrated on a rotary evaporator under vacuum and the residue was purified by flash chromatography on 5 g of silica gel 60 (10×110 mm column) using ethyl acetate as eluant to yield the product. MS (ESI): m/z 237 (25%, MH⁺ for product), 181 (100%,-56,-isobutylene).

This method would also apply to the preparation of (S)-tert-Butyl-2-amino-1-phenylethylcarbamate from the corresponding enantiomeric starting material.

### Example 3

### Synthesis of (S)-2-amino-2-phenylethyl-5-methyl-2-(1-methylethyl)-cyclohexanecarboxamide

Option A Synthesis of (1R,2S,5R)-N-((S)-2-amino-2-phenylethyl)-2-isopropyl-5-methylcyclohexane-1-carboxamide via Carbodiimide Coupling

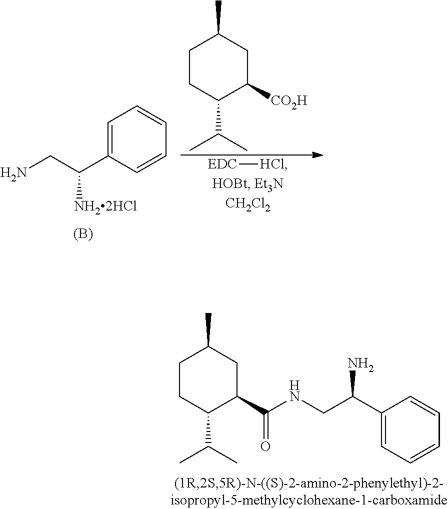

In a 250 mL 24/40 joint round bottom flask equipped with an N₂ inlet was added solid (1R,2S,5R)-2-isopropyl-5-methylcyclohexane-1-carboxylic acid (1 g, 5.43 mmol). Anhydrous CH₂Cl₂ (100 mL) under N₂ atmosphere was added via cannula. The reaction was cooled to 0° C. with an ice-water bath. Hydroxybenzotriazole powder (HOBt) (1.1. g, 8.15 mmol), and solid (S)-1-phenylethane-1,2-diamine dihydrochloride salt (B) (1.25 g, 5.97 mmol) were added with a spatula. Triethylamine (2.3 mL, 16.29 mmol) was then added drop wise over 10 minutes with a syringe. Finally, 3-(ethyliminomethylideneamino)-N,N-dimethyl EDC.HCl (1.35 g, 7.05 mmol) was added with a spatula, the ice-bath was removed, and the reaction was warmed to (RT) over 2 hr. The reaction was stirred with a stirring bar overnight and then saturated NaCl solution (200 mL) was added. The resulting organic and aqueous layers were separated in a separatory funnel and the organic layer was washed with saturated NaCl solution (100 mL) and then dried over anhydrous Na₂SO₄ for 1 hr. The organic layer was concentrated under vacuum (Buchi Rotovapor R-124, 5-10 mm Hg) and the residue was purified by column chromatography (SiO₂, 3% MeOH-CH2Cl2) to provide (1R,2S ,5R)-N-((S)-2-amino-2-phenylethyl)-2-isopropyl-5-methylcyclohexane-1-carboxamide

300 mg; MS (ESI): 302.

Option B Synthesis of (1R,2S,5R)-N-((S)-2-amino-2-phenylethyl)-2-isopropyl-5-methylcyclohexane-1-carboxamide (via Acid Chloride Coupling)

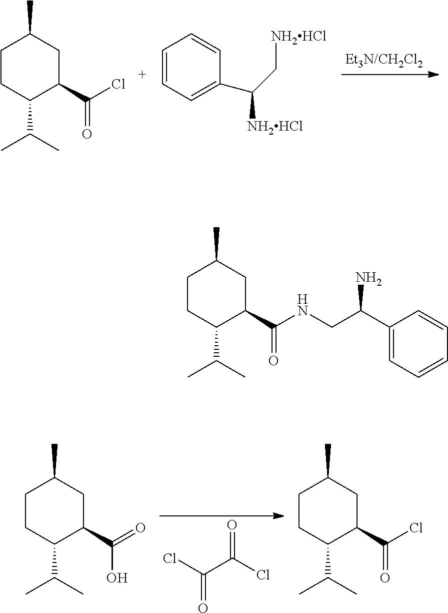

(1R,2S,5R)-2-Isopropyl-5-methylcyclohexane-1-carbonyl chloride

A 250 ml single neck round bottom flask was charged with (1R,2S,5R)-2-Isopropyl-5-methylcyclohexanecarboxylic acid (10.001 gram, 0.054 mol), and 92 ml of oxalyl chloride (138 g, 1.08 mol). The solution was stirred under a positive pressure nitrogen atmosphere for 18 hours then concentrated under vacuum. Product was recovered as 10.42 grams of a clear, colorless liquid.

Coupling Procedure

In a 300 mL 2-neck round bottom flask equipped with stir bar, N₂ inlet for inert gas and an additional funnel was dissolved (1R,2S,5R)-2-isopropyl-5-methylcyclohexane-1-carbonyl chloride (0.882 g, 4.3 mmol) in anhydrous CH₂Cl₂ (100 mL) under N₂ atmosphere. The reaction was cooled to 0° C. with an ice-water bath and solid (S)-1-phenylethane-1,2-diamine dihydrochloride salt was added in a single portion via spatula (1 g, 4.8 mmol) while Et₃N (2.5 mL) simultaneously was added via the addition funnel. The reaction was maintained at 0° C. (ice bath) for 1 hr, then the bath was removed and the reaction was warmed to RT and stirred with a stir bar overnight. The reaction was quenched by pouring it into a 500 mL separatory funnel containing saturated NaCl solution (100 mL). The layers were separated, and the aqueous layer was extracted with CH₂Cl₂ (3×100 mL). The organic layers were combined in a 1L Erlenmeyer flask and then dried over anhydrous Na₂ SO₄ for 1 hr. The drying agent was removed by gravity filtration and the filtrate was concentrated under vacuum (Buchi Rotovapor R-124, 5-10 mm Hg), to provide an off-white solid. The solid was chromatographed (SiO₂, 5%MeOH-CH₂Cl₂) to provide (1R,2S,5R)-N-((S)-2-amino-2-phenylethyl)-2-isopropyl-5-methylcyclohexane-1-carboxamide (C); 250 mg. MS(ESI): 302.

Option C Synthesis of (1R,2S,5R)-N-((S)-2-amino-2-phenylethyl)-2-isopropyl-5-methylcyclohexane-1-carboxamide via Acid Chloride Coupling of Protected (S)-1-phenylethane-1,2-diamine

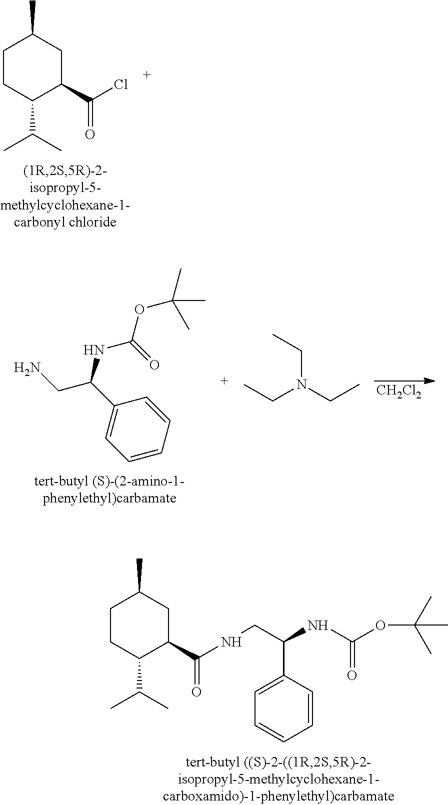

tert-Butyl((S)-2-((1R,2S,5R)-2-isopropyl-5-methylcyclohexane-1-carboxamido)-1-phenylethyl)carbamate

A 3-neck round bottom flask equipped with a side-arm addition funnel, condenser with outlet to a Firestone valve (positive argon pressure), and magnetic stir bar was charged with (1R,2S,5R)-2-isopropyl-5-methylcyclohexane-1-carbonyl chloride (2.0273 grams, 0.010 mol) and dissolved in CH₂Cl₂ (14 mL) with stirring in an ice water bath under argon. (S)-tert-Butyl-2-amino-1-phenylethylcarbamate (2.3633 grams, 0.010 mol) made by conventional methods from compound B was dissolved in 10 mL of CH₂Cl₂ and the solution was transferred to an addition funnel making a complete transfer with 3×1.0 mL of CH₂Cl₂. Triethylamine (1.0604 g, 0.0105 mol) was added to the same addition funnel. The combined solution of amines was added drop-wise to the reaction flask at a rate to maintain a reaction temperature near 5° C. The addition funnel was rinsed with 3×0.5 mL of CH₂Cl₂. The ice water bath was removed and stirring was continued at room temperature. An additional 5 mL of CH₂Cl₂ was added and stirring was continued at room temperature under argon overnight. The reaction mixture was transferred to a separatory funnel along with 3×5 mL rinses of CH₂Cl₂. The CH₂Cl₂ solution was extracted with 1N HCl solution (2×50 mL), distilled H₂O (3×75 mL), dried over anhydrous magnesium sulfate, vacuum filtered, and concentrated under vacuum at 28° C. The product was recovered as a white powder (3.1445 g); MS (ESI) m/z 403 (MH⁺).

(1R,2S,5R)-N-((S)-2-Amino-2-phenylethyl)-2-isopropyl-5-methylcyclohexane-1-carboxamide

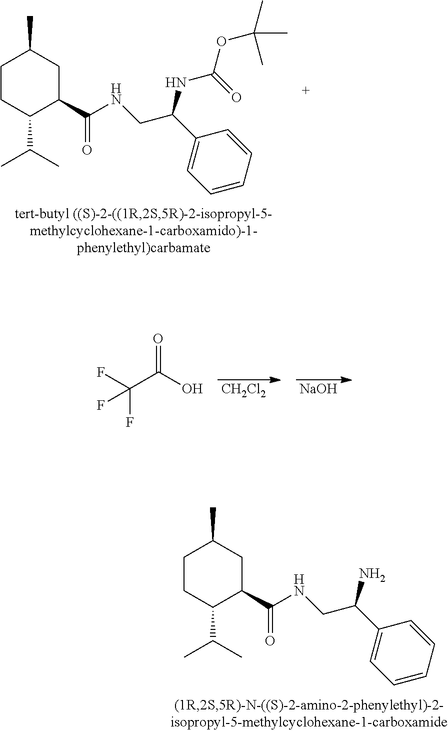

A 100 mL single neck round bottom flask was charged with 1.207 g (3.0 mmol) of tert-butyl ((S)-2-((1R,2S ,5R)-2-isopropyl-5-methylcyclohexane-1-carboxamido)-1-phenylethyl)carbamate and 40 mL of anhydrous dichloromethane. The homogenous solution was magnetically stirred under positive nitrogen pressure. Trifluoroacetic acid (6.0 mL, 8.94 g, 78 mmol) was added to the reaction flask and stirring was continued for 80 minutes. The reaction mixture was slowly added to 100 mL of 1 M NaOH solution in a 250 mL separatory funnel over a 20 minute period, mixing occasionally to avoid dichloromethane boil off (heat generation). The reaction flask was rinsed with 3×2 mL CH₂Cl₂ and added to the separatory funnel to make a complete transfer. The mixture was shaken repeatedly and the two phases allowed to separate. The lower organic phase was removed and the aqueous phase extracted 1×50 mL of CH₂Cl₂. The organic layers were combined and washed 2×25 mL of saturated sodium chloride solution. The organic phase was dried over anhydrous sodium sulfate for 30 min, vacuum filtered, and concentrated on a rotary evaporator (maximum bath temp 38° C.). The product (0.812 g) was recovered as a light beige colored solid. MS(ESI) m/z 303 (MH⁺).

### Example 4

### Synthesis of (1R,2S,5R)-N-((R)-2-Amino-2-phenylethyl)-2-isopropyl-5-methylcyclohexane-1-carboxamide

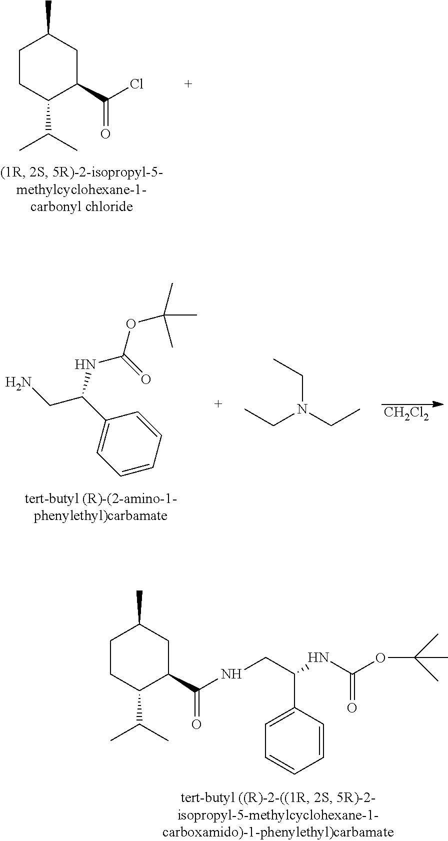

This material was prepared in the same manner as described for tert-butyl ((S)-2-((1R,2S,5R)-2-isopropyl-5-methylcyclohexane-1-carboxamido)-1-phenylethyl)carbamate (EXAMPLE 3; Option C) starting from (R)-tert-Butyl-2-amino-1-phenylethylcarbamate. MS(ESI) m/z 403 (MH⁺). (1R,2S,5R)-N-((R)-2-Amino-2-phenylethyl)-2-isopropyl-5-methylcyclohexane-1-carboxamide

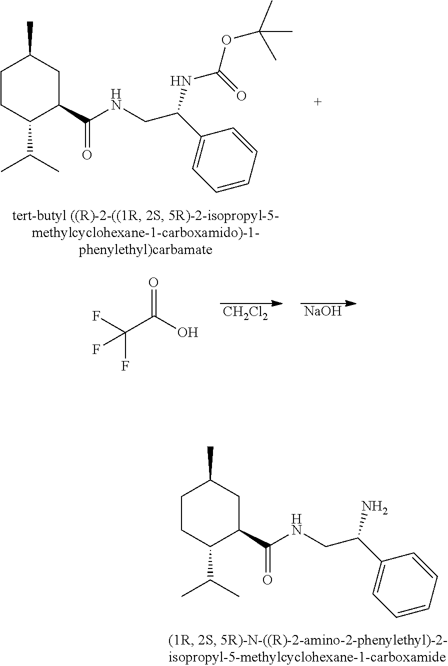

This material was prepared in the same manner as described for (1R,2S,5R)-N-((S)-2-amino-2-phenylethyl)-2-isopropyl-5-methylcyclohexane-1-carboxamide (EXAMPLE 3; Option C) starting from tert-butyl ((R)-2-((1R,2S,5R)-2-isopropyl-5-methylcyclohexane-1-carboxamido)-1-phenylethyl)carbamate. MS(ESI) m/z 303 (MH⁺).

### Example 5

### Synthesis of (1R,2S,5R)-N-((S)-2-acetamido-2-phenylethyl)-2-isopropyl-5-methylcyclohexane-1-carboxamide

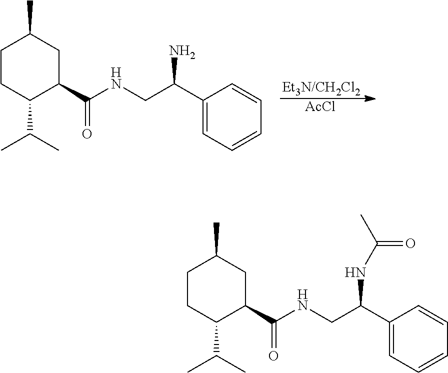 [SMILES: "AcCl" ; CC(C)[C@@H]1CC[C@@H](C)C[C@H]1C(=O)NC[C@@H](N)c1ccccc1 ; CC(=O)N[C@H](CNC(=O)[C@@H]1C[C@H](C)CC[C@H]1C(C)C)c1ccccc1]

To a solution of (1R,2S,5R)-N-((S)-2-amino-2-phenylethyl)-2-isopropyl-5-methylcyclohexane-1-carboxamide (100 mg, 0.331 mmol, 1 equiv.) in anhydrous CH₂Cl₂ (20 mL) cooled 0° C. was added trimethylamine (70 μL. 0.500 mmol, 1.5 equiv) via syringe. The reaction was stirred 10 minutes at 0° C. and then acetyl chloride (90 uL, 0.403 mmol, 1.2 equiv) was added dropwise over 10 minutes via syringe. The reaction was warmed to RT and stirred overnight. The reaction was poured onto saturated aqueous NaHCO₃ and extracted 3×100 mL CH₂Cl₂ using a separatory funnel. The organic layers were combined, dried over anhydrous Na₂SO₄, filtered to remove drying agent, and concentrated under vacuum to give an orange solid. This material was purified by column chromatography (SiO₂, 50/50 hexanes/EtOAc) to provide a yellow solid; 10 mg. MS/ESI (M⁺H⁺): 345.

### Example 6

### Synthesis of (1R,2S,5R)-2-isopropyl-5-methyl-N-((S)-2-palmitamido-2-phenylethyl)cyclohexane-1-carboxamide

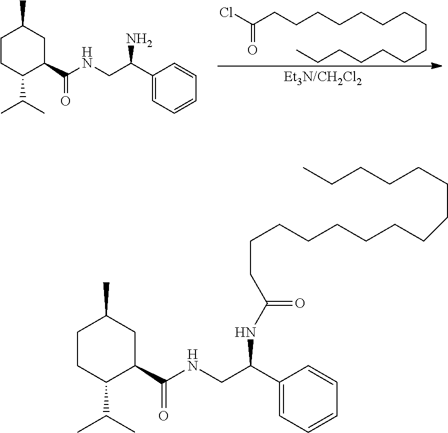 [SMILES: CC(C)[C@@H]1CC[C@@H](C)C[C@H]1C(=O)NC[C@@H](N)c1ccccc1 ; CCCCCCCCCCCCCCCC(=O)Cl ; CCCCCCCCCCCCCCCC(=O)N[C@H](CNC(=O)[C@@H]1C[C@H](C)CC[C@H]1C(C)C)c1ccccc1]

To a solution of (1R,2S,5R)-N-((S)-2-amino-2-phenylethyl)-2-isopropyl-5-methylcyclohexane-1-carboxamide (C) (180 mg, 0.596 mmol, 1 equiv), in CH₂Cl₂ (20 mL) at 0° C. (ice bath) was added Et₃N (100 uL, 1.35 mmol, 2.35 equiv.), followed by palmitoyl chloride (180 mg, 0.892 mmol, 1.5 equiv.). The reaction was warmed to RT and stirred overnight. Saturated aqueous NaHCO₃ was added and the layers were separated. The organic layer was separated using a separatory funnel, dried (Na₂SO₄), filtered to remove drying agent, and then the solvent was removed under vacuum (5 mm Hg) using a rotary evaporator. The solid was crystallized from acetone/MeOH to give the product as a white solid; 185 mg.

MS (ESI/M⁺H⁺): 541.

### Example 7

### Synthesis of (1R,2S,5R)-N-((S)-2-isopropyl-5-methylcyclohexane-1-carboxamide-1-phenyethyl)benzamide

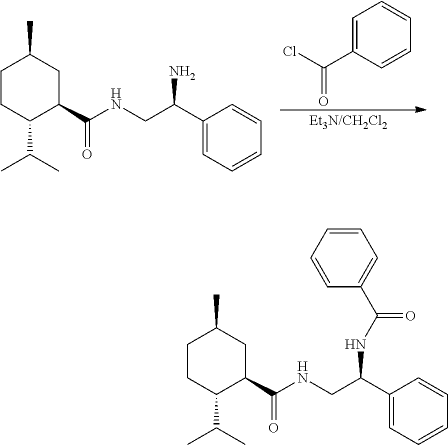 [SMILES: CC(C)[C@@H]1CC[C@@H](C)C[C@H]1C(=O)NC[C@@H](N)c1ccccc1 ; O=C(Cl)c1ccccc1 ; CC(C)[C@@H]1CC[C@@H](C)C[C@H]1C(=O)NC[C@@H](NC(=O)c1ccccc1)c1ccccc1]

A solution of (1R,2S,5R)-N-((S)-2-amino-2-phenylethyl)-2-isopropyl-5-methylcyclohexane-1-carboxamide (C) (180 mg, 0.596 mmol, 1 equiv.) in CH₂Cl₂ (20 mL) was cooled to 0° C. with an ice bath. Et₃N (0.250 mL, 1.79 mmol, 3 equiv.) was added dropwise via syringe and stirred 10 minutes. To this solution was added benzoyl chloride (92 mg, 0.655 mmol, 1.1 equiv) via syringe over 10 minutes. The reaction was stirred overnight, then poured onto 100 mL CH₂Cl₂/10 mL10% HCl and extracted 3×50 mL with CH₂Cl₂ using a separatory funnel. The organic layers were separated, combined, washed with saturated NaCl solution (100 mL), dried (Na₂SO₄), filtered to remove drying agent, and then concentrated under vacuum using a rotary evaporator. The resulting solid was triturated with hexanes, and the crystals were collected via filtration using a 60 mL Buchner funnel. The resulting off-white solid was dried overnight under house vacuum (5-10 mmHg) to provide 65 mg of the final product; MS/ESI (M+H+): 407.

### Example 8

### Synthesis of (1R,2S,5R)-N-((S)-2-((R)-2-Aminopropanamido)-2-phenylethyl)-2-isopropyl-5-methylcyclohexane-1-carboxamide

tert-Butyl ((R)-1-(((S)-2-((1R,2S,5R)-2-isopropyl-5-methylcyclohexane-1-carboxamido)-1-phenylethyl)amino)-1-oxopropan-2-yl)carbamate

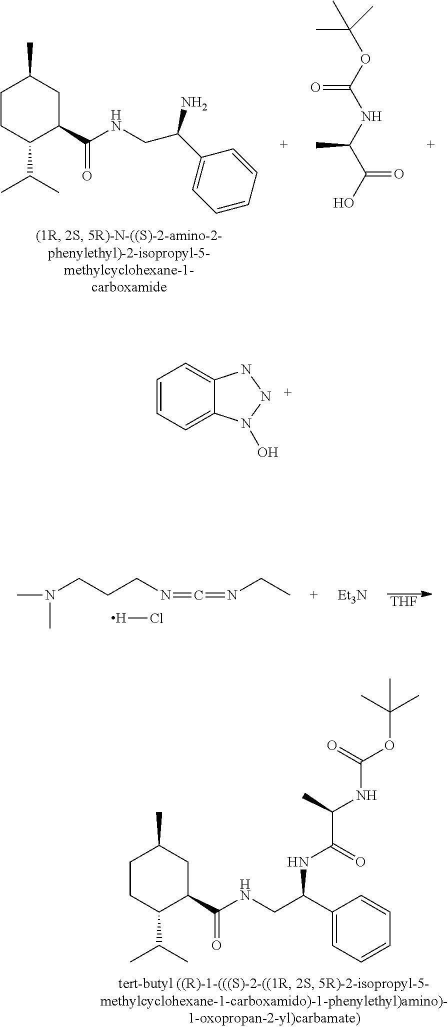

A 250 mL 3-neck round bottom flask equipped with a condenser with an outlet to a Firestone valve (positive nitrogen pressure) and a magnetic stir bar was charged with Boc-D-Ala (0.511 g, 2.70 mmol), HOBt (0.364 g, 2.69 mmol), EDC-HCl (0.520 g, 2.71 mmol), and 95 mL anhydrous tetrahydrofuran. The solution was stirred at room temperature under nitrogen and triethylamine (380 μL, 0.273 g, 2.70 mmol) was added. To the heterogeneous mixture was added (1R,2S,5R)-N-((S)-2-amino-2-phenylethyl)-2-isopropyl-5-methylcyclohexane-1-carboxamide (0.741 g, 2.45 mmol) dissolved in 40 mL of THF. The heterogeneous mixture was stirred under a nitrogen atmosphere for 3 hours. The reaction mixture was transferred to a 1L separatory funnel containing ethyl acetate (100 mL) and water (100 mL). The aqueous layer was separated and extracted again with 2×80 mL of ethyl acetate. The combined organic phases were washed with 1N HCl solution (2×50 mL), H₂O (1×50 mL), saturated sodium bicarbonate solution (3×50 mL), and brine (1×50 mL). The solution was dried over anhydrous sodium sulfate, filtered, and concentrated under vacuum at 38° C. to give 1.13 grams of a white solid; MS (ESI) m/z 474 (100%,MH⁺), 418 (25%), 374 (10%).

(1R,2S,5R)-N-((S)-2-((R)-2-Aminopropanamido)-2-phenylethyl)-2-isopropyl-5-methylcyclohexane-1-carboxamide

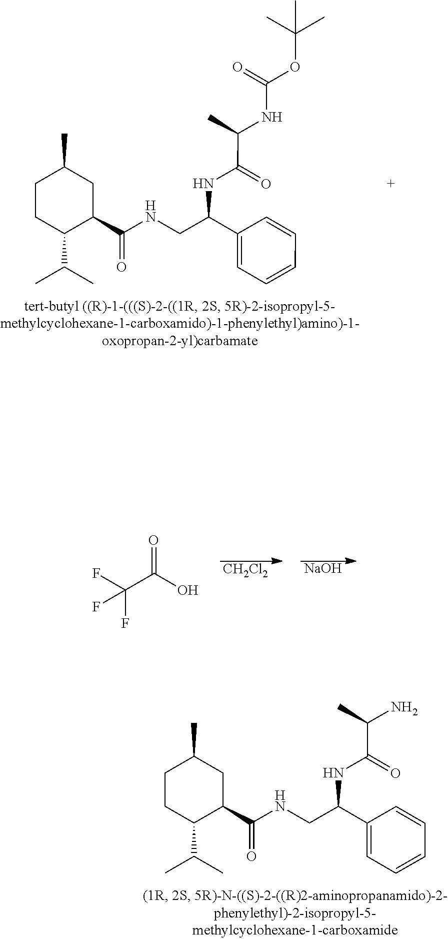

A 250 mL 3-neck round bottom flask equipped with a magnetic stir bar, condenser with outlet to a Firestone valve (positive nitrogen pressure), and a magnetic stir bar was charged with (0.534 grams, 1.12 mmol) of tert-butyl ((R)-1-(((S)-2-((1R,2S ,5R)-2-isopropyl-5-methylcyclohexane-1-carboxamido)-1-phenylethyl)amino)-1-oxopropan-2-yl)carbamate, 80 mL of anhydrous dichloromethane, and 6 mL of trifluoroacetic acid. The solution was stirred at room temperature under a nitrogen atmosphere for 95 minutes. The reaction solution was added slowly to a separatory funnel containing 100 mL of 1N NaOH solution over a 25 minute period. The mixture was shaken repeatedly and the phases were allowed to separate. The lower organic phase was removed and the aqueous phase extracted with another 50 mL of CH₂Cl₂. The organic layers were combined and washed with 2×25 mL of saturated sodium chloride solution, dried over anhydrous sodium sulfate, vacuum filtered, and concentrated under vacuum at 38° C. to provide 0.42 grams of the product as a white solid; MS(ESI) m/z 374 (MH⁺).

### Example 9

### tert-Butyl ((R)-1-(((R)-2-((1R,2S ,5R)-2-isopropyl-5-methylcyclohexane-1-carboxamido)-1-phenylethyl)amino)-1-oxopropan-2-yl)carbamate

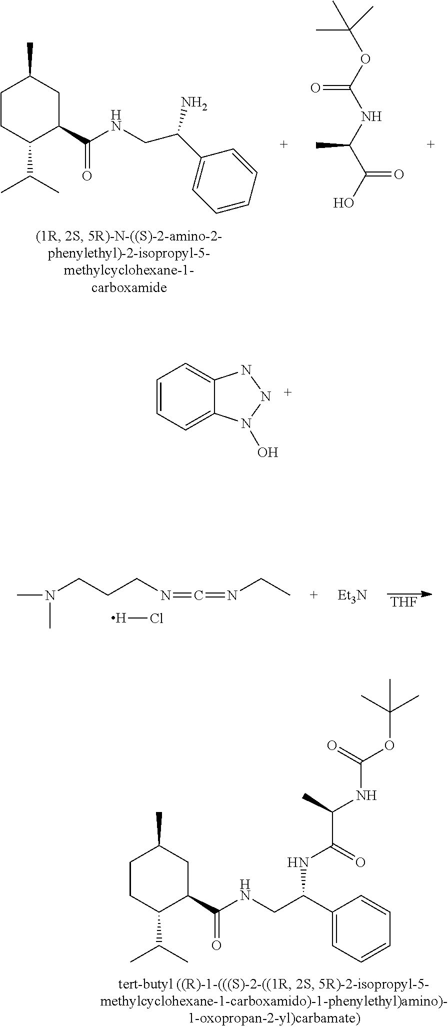

This material was prepared in the same manner as described for tert-Butyl ((R)-1-(((S)-2-((1R,2S ,5R)-2-isopropyl-5-methylcyclohexane-1-carboxamido)-1-phenylethyl)amino)-1-oxopropan-2-yl)carbamate (EXAMPLE 8) starting from (1R,2S,5R)-N-((R)-2-amino-2-pheylethy)-2-isopropyl-5-methylcyclohexane-1-carboxamide MS(ESI) m/z 474.5 (20%), 418 (40%), 374.4 (100%).

(1R,2S ,5R)-N-((R)-2-((R)-2-Aminopropanamido)-2-phenylethyl)-2-isopropyl-5-methylcyclohexane-1-carboxamide

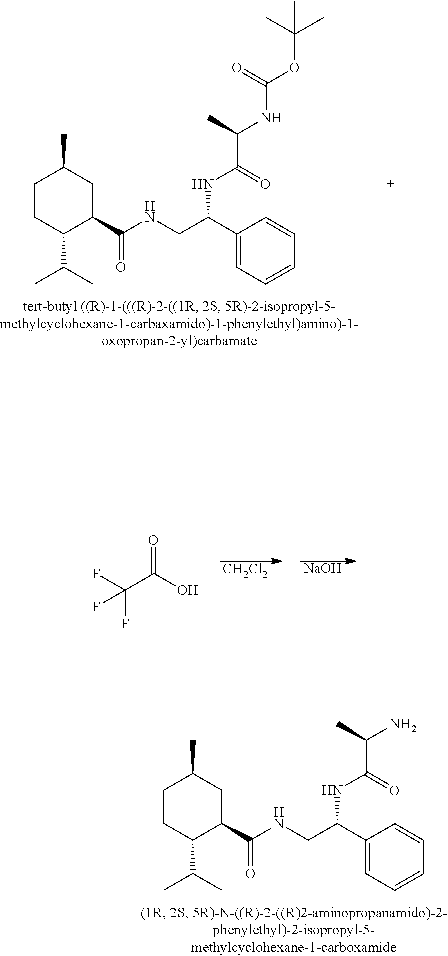

This material was prepared in the same manner as described for (1R,2S,5R)-N-((S)-2-((R)-2-Aminopropanamido)-2-phenylethyl)-2-isopropyl-5-methylcyclohexane-1-carboxamide (EXAMPLE 8) starting with tert-butyl ((R)-1-(((R)-2-((1R,2S,5R)-2-isopropyl-5-methylcyclohexane-1-carboxamido)-1-phenylethyl)amino)-1-oxopropan-2-yl)carbamate. MS(ESI) m/z 374 (MH⁺).

### Example 10

### Synthesis of (1R,2S,5R)-N-((S)-2-((S)-2-aminopropanamido)-2-phenylethyl)-2-isopropyl-5-methylcyclohexane-1-carboxamide

tert-butyl ((S)-1-(((S)-2-((1R,2S,5R)-2-isopropyl-5-methylcyclohexane-1-carboxamido)-1-phenylethyl)amino)-1-oxopropan-2-yl)carbamate

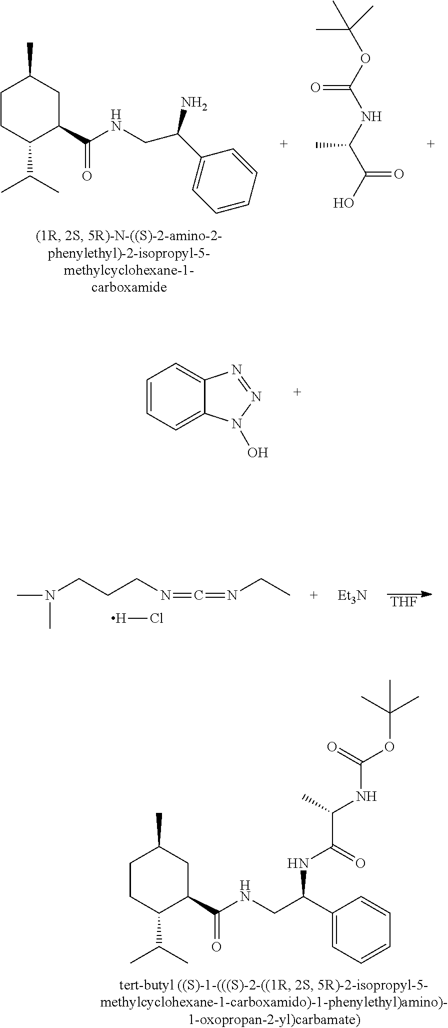

This material was prepared in the same manner as described for tert-Butyl ((R)-1-(((S)-2-((1R,2S,5R)-2-isopropyl-5-methylcyclohexane-1-carboxamido)-1-phenylethyl)amino)-1-oxopropan-2-yl)carbamate (EXAMPLE 8) using Boc-L-Ala in place of Boc-D-Ala. MS(ESI) 474.5 (MH+).

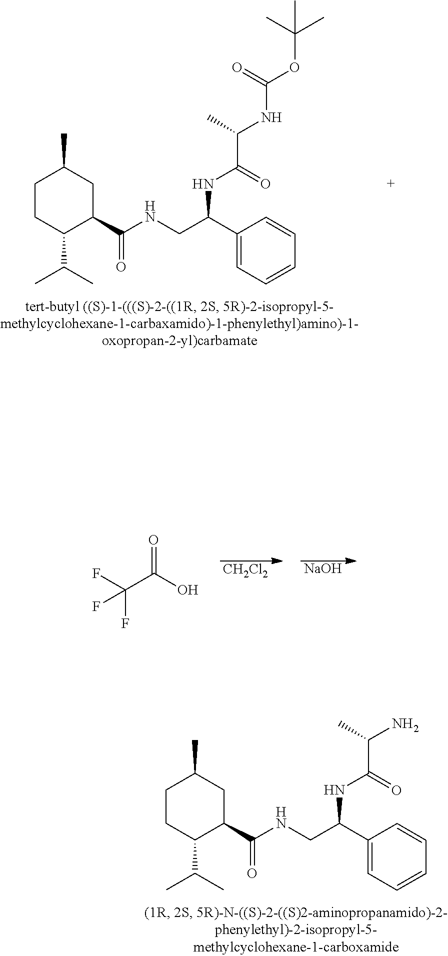

(1R,2S,5R)-N-((S)-2-((S)-2-aminopropanamido)-2-phenylethyl)-2-isopropyl-5-methylcyclohexane-1-carboxamide

This material was prepared in the same manner as described for (1R,2S,5R)-N-((S)-2-((R)-2-aminopropanamido)-2-phenylethyl)-2-isopropyl-5-methylcyclohexane-1-carboxamide starting with tert-butyl ((S)-1-(((S)-2-((1R,2S,5R)-2-isopropyl-5-methylcyclohexane-1-carboxamido)-1-phenylethyl)amino)-1-oxopropan-2-yl)carbamate. MS(ESI) m/z 374 (MH⁺).

### Example 11

### Synthesis of (1R,1′R,2S,2′S,5R,5′R)-N,N′-((S)-1-phenylethane-1,2-diyl)bis(2-isopropyl-5-methylcyclohexane-1-carboxamide)

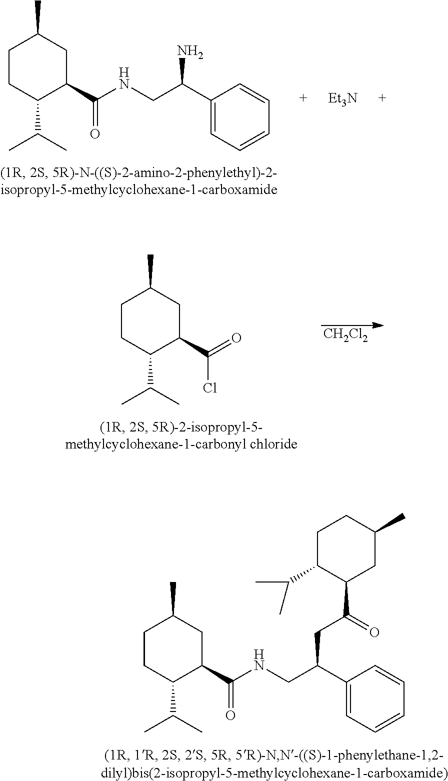 [SMILES: "Et3N" ; CC(C)[C@@H]1CC[C@@H](C)C[C@H]1C(=O)NC[C@@H](N)c1ccccc1 ; CC(C)[C@@H]1CC[C@@H](C)C[C@H]1C(=O)Cl ; "CH2Cl2" ; CC(C)[C@@H]1CC[C@@H](C)C[C@H]1C(=O)C[C@H](CNC(=O)[C@@H]1C[C@H](C)CC[C@H]1C(C)C)c1ccccc1]

A 3-neck 15 mL round bottom flask equipped with a magnetic stir bar and a condenser with outlet to a Firestone valve (positive nitrogen pressure) was charged with (1R,2S,5R)-2-isopropyl-5-methylcyclohexane-1-carbonyl chloride (0.0813 g, 0.4010 mmol) and diluted with 1 mL of CH₂Cl₂. The mixture was stirred in an ice water bath under a nitrogen atmosphere. The triethylamine (0.0429 g, 0.424 mmol) and (1R,2S,5R)-N-((S)-2-amino-2-phenylethyl)-2-isopropyl-5-methylcyclohexane-1-carboxamide (0.1213 g, 0.4010 mmol) were combined in 2-3 mL of CH₂Cl₂. This solution was slowly added to the acid chloride solution while keeping the reaction flask in the ice bath during the addition. After the addition was complete the ice bath was removed and the reaction was stirred at room temperature. The reaction mixture was diluted with CH₂Cl₂ (12 mL) to provide a homogenous solution and stirred at room temperature under nitrogen for 16 hours. The homogenous reaction solution was transferred to a 50 mL separatory funnel making a complete transfer with CH₂Cl₂ rinses (3×2 mL). The CH₂Cl₂ solution was washed with 1N HCl solution (2×25 mL), distilled water (3×20 mL), 1 N NaOH solution (2×25 mL), distilled water (2×50 mL), and then dried over anhydrous MgSO₄, vacuum filtered, and concentrated under vacuum (38° C.) to obtain the product as 0.1131 g of a white solid; MS(ESI) m/z 469 (MH⁺).

### Example 12

### Synthesis of (1R,2S,5R)-2-isopropyl-N-((R)-1-(((S)-2-((1R,2S,5R)-2-isopropyl-5-methylcyclohexane-1-carboxamido)-1-phenylethyl)amino)-1-oxopropan-2-yl)-5-methylcyclohexane-1-carboxamide

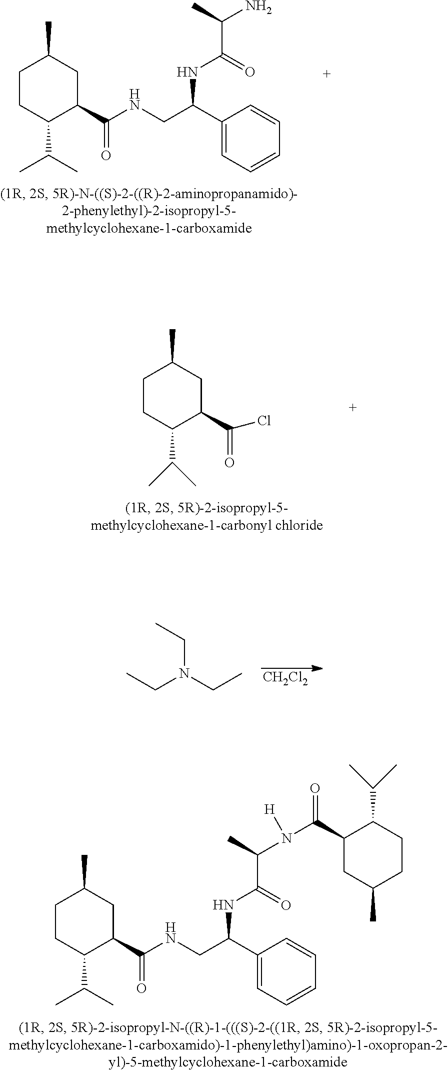

A single neck 35 mL round bottom flask equipped with a magnetic stir bar and an addition funnel with outlet to a Firestone valve (positive nitrogen pressure) was charged with 0.0488 g (0.241 mmol) of (1R,2S,5R)-2-isopropyl-5-methylcyclohexane-1-carbonyl chloride and diluted with 1.5 mL of CH₂Cl₂. The solution was stirred in an ice water bath under a nitrogen atmosphere. The addition funnel was charged with (1R,2S,5R)-N-((S)-2-((R)-2-aminopropanamido)-2-phenylethyl)-2-isopropyl-5-methylcyclohexane-1-carboxamide (0.090 g, 0.241 mmol) and triethylamine (0.0256 g, 0.253 mmol) in 3 mL of CH₂Cl₂. This solution was slowly added to the acid chloride solution while keeping the reaction flask in the ice bath during the addition. A complete transfer was made by rinsing the addition funnel (3×0.5 mL CH₂Cl₂). The ice bath was removed and the mixture stirred. An additional 2 mL CH₂Cl₂ was added to the mixture and stirring continued overnight under a nitrogen atmosphere. The mixture was transferred to a separatory funnel making a complete transfer with CH₂Cl₂ (3×5 mL). The CH₂Cl₂ solution was washed with 1N HCl soln (2×50 mL), H₂O (1×25 mL), 1 N NaOH (2×50 mL), and distilled H₂O (2×50 mL) then dried over anhydrous sodium sulfate, vacuum filtered, and concentrated under vacuum (38° C.) to obtain the product as a white powder 0.1336 g. MS(ESI) m/z 540 (MH⁺) .

### Example 13

### Synthesis of (1R, 1′R,2S,2′S,5R,5′R)-N,N’-(ethane-1,2-diyl)bis(2-isopropyl-5-methylcyclohexane-1-carboxamide)

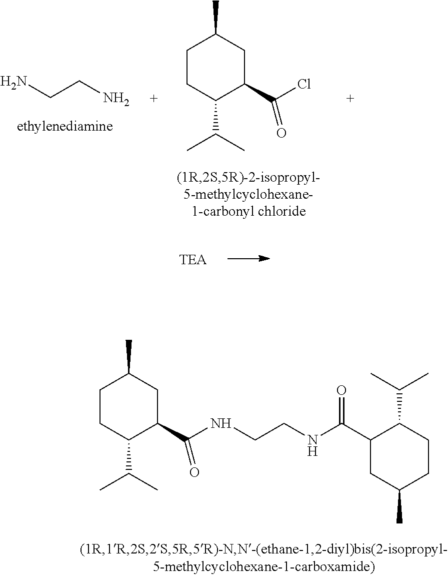

A 25 ml 3-neck round bottom flask equipped with a magnetic stir bar, a 10 mL addition funnel, and condenser were charged with (1R,2S,5R)-2-isopropyl-5-methylcyclohexane-1-carbonyl chloride (1.0001 g, 4.93 mmol), diluted with 7 mL of anhydrous dichloromethane and stirred in an ice bath under nitrogen atmosphere. The addition funnel was charged with ethylenediamine (0.1505 g, 2.49 mmol) and triethylamine (0.524 g, 5.18 mmol) in 4.5 mL of CH₂Cl₂. The combined amine solution was added dropwise to the reaction flask keeping the reaction mixture cold during the addition. The addition funnel was rinsed with 1 mL of CH₂Cl₂ and the ice bath removed. The mixture was stirred overnight at room temperature under a nitrogen atmosphere. The mixture was transferred to a separatory funnel, making a complete transfer with 4×10 ml rinses of CH₂Cl₂. The organic phase was washed with 1N HCl solution (3×50 mL), then distilled H₂O (3×50 mL) and dried over anhydrous magnesium sulfate overnight, vacuum filtered, and concentrated under vacuum to recover product as a white powder (0.7318 g); MS(ESI) m/z 393 (MH⁺).

### Example 14

### Synthesis of (1R,2S,5R)-N-((S)-2-(2-aminoacetamido)-2-phenylethyl)-2-isopropyl-5-methylcyclohexane-1-carboxamide

tert-Butyl (2-(((S)-2-((1R,2S,5R)-2-isopropyl-5-methylcyclohexane-1-carboxamido)-1-phenylethyl)amino)-2-oxoethyl)carbamate

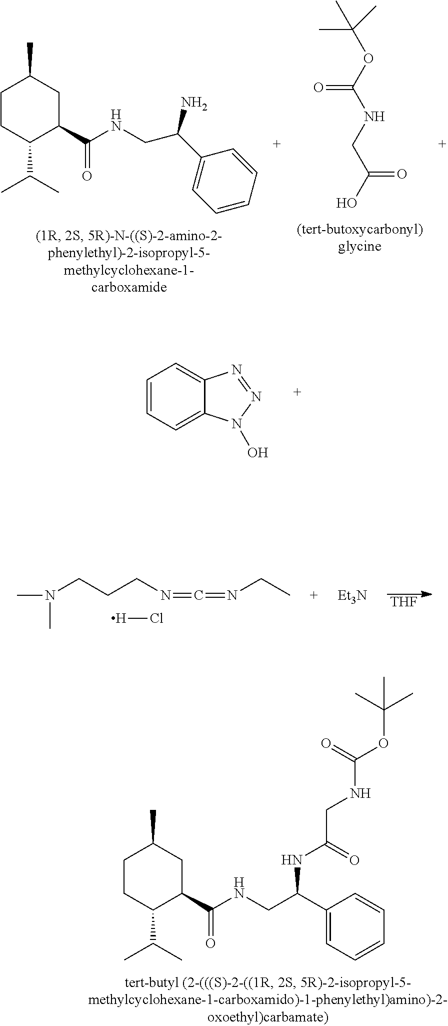

This material was prepared in the same manner as described for tert-Butyl ((R)-1-(((S)-2-((1R,2S ,5R)-2-isopropyl-5-methylcyclohexane-1-carboxamido)-1-phenylethyl)amino)-1-oxopropan-2-yl)carbamate (EXAMPLE 8) using Boc-Glycine in place of Boc-D-Ala; MS(ESI) 460 (MH⁺).

(1R,2S,5R)-N-((S)-2-(2-aminoacetamido)-2-phenylethyl)-2-isopropyl-5-methylcyclohexane-1-carboxamide

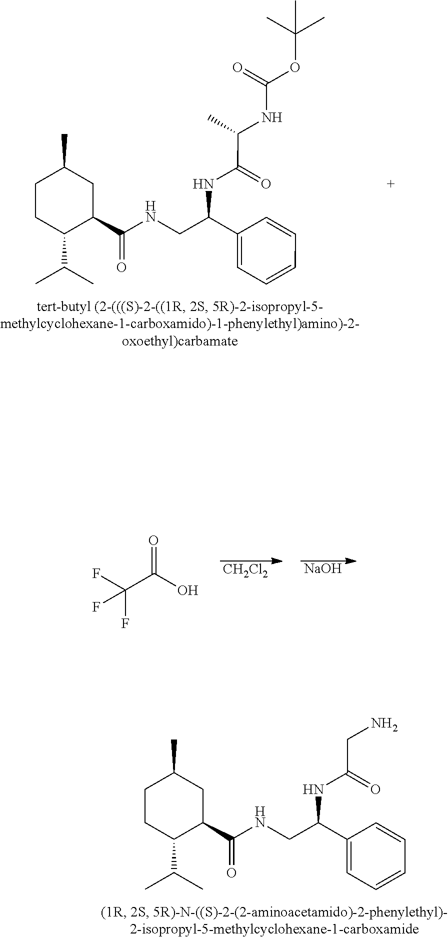

This material was prepared in the same manner as described for (1R,2S,5R)-N-((S)-2-((R)-2-aminopropanamido)-2-phenylethyl)-2-isopropyl-5-methylcyclohexane-1-carboxamide (EXAMPLE 8) starting with tert-butyl (2-(((S)-2-((1R,2S,5R)-2-isopropyl-5-methylcyclohexane-1-carboxamido)-1-phenylethyl)amino)-2-oxoethyl)carbamate; MS(ESI) 360 (MH⁺).

### Example 15

### Synthesis of (1R,2S,5R)-N-((S)-2-hydroxy-2-phenylethyl)-2-isopropyl-5-methylcyclohexane-1-carboxamide

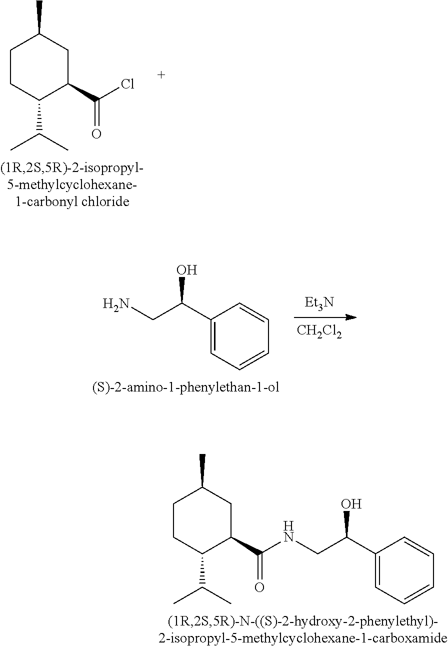

To a dry, 25 mL, round-bottom flask (Flask A) was added 0.4961 grams (3.62 mmols) of (S)-2-amino-1-phenylethan-1-ol, 0.689 grams (6.81 mmols) triethylamine and 5 ml of anhydrous methylene chloride. The reaction flask was immersed in an ice bath. To a separate dry, 25 mL, round-bottom flask (Flask B) was added 0.597 grams (2.95 mmols) of (1R,2S,5R)-2-Isopropyl-5-methylcyclohexane-1-carbonyl chloride and 5 mL of anhydrous methylene chloride. The contents of flask B were added to a dry, 25 mL, pressure-equalizing addition funnel. The addition funnel was connected to the reaction flask A and the solution of acid chloride was added slowly while mixing at 300 r.p.m. over ten minutes and while purging the apparatus head space with dry nitrogen. The addition funnel was then rinsed with 4 mL of anhydrous methylene chloride which was added to the reaction flask. The reactor contents were allowed to continue to mix for an additional 5 hours at 300 r.p.m. under melting ice bath conditions and a static, dry nitrogen atmosphere. Following the reaction period, the reaction solution was added to a 250 mL separatory funnel and diluted to a total volume of 75 mL with 60 mL of anhydrous diethyl ether. The organic layer was extracted with three 20 mL aliquots of 1.0 N HCl, two 20 mL aliquots of saturated sodium bicarbonate, one 20 mL aliquot of distilled water and one 20 mL aliquot of saturated sodium chloride. The extracted organic layer was dried over anhydrous sodium sulfate overnight, filtered through Whatman #4 filter to remove any particulates and then the solvent was removed in vacuo to yield 0.7 grams of the title compound; MS(ESI) m/z 304 (MH⁺).

### Example 16 and Example 17

### Syntheses of the isomeric materials (1R,2S,5R)-N-((R)-2-hydroxy-2-phenylethyl)-2-isopropyl-5-methylcyclohexane-1-carboxamide and (1R,2S,5R)-N-((R/S)-2-hydroxy-2-phenylethyl)-2-isopropyl-5-methylcyclohexane-1-carboxamide

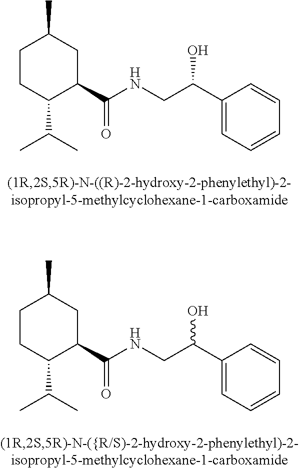 [SMILES: CC(C)[C@@H]1CC[C@@H](C)C[C@H]1C(=O)NC[C@H](O)c1ccccc1 ; CC(C)[C@@H]1CC[C@@H](C)C[C@H]1C(=O)NCC(O)c1ccccc1]

These materials were prepared in the same manner as EXAMPLE 15 starting with (R)-2-amino-1-phenylethan-1-ol and racemic (R/S)-2-amino-1-phenylethan-1-ol, respectively.

### Example 18

### Synthesis of (S)-2-((1R,2S,5R)-2-isopropyl-5-methylcyclohexane-1-carboxamido)-1-phenylethyl (tert-butoxycarbonyl)glycinate

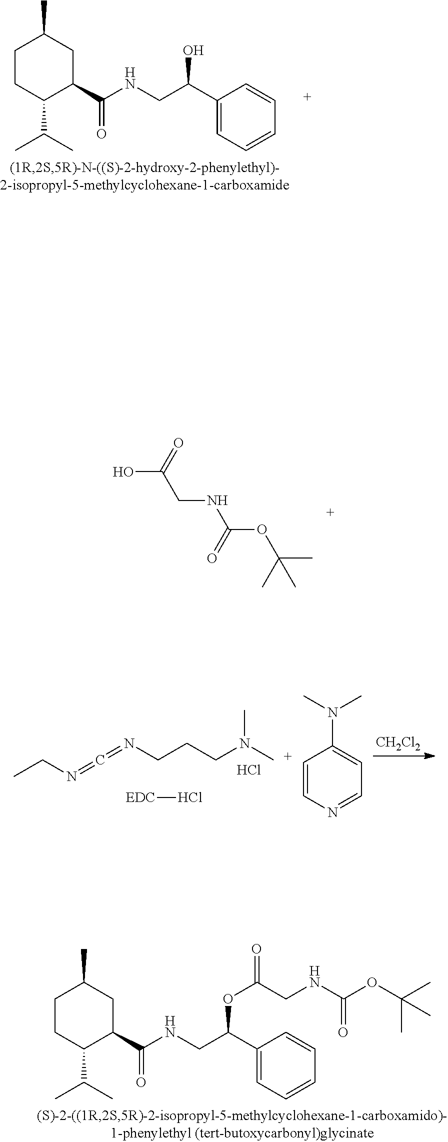

A 50 mL, round-bottom flask (Flask A) was charged with 0.260 grams (0.86 mmols) of the starting (1R,2S ,5R)-N-((S)-2-hydroxy-2-phenylethyl)-2-isopropyl-5-methylcyclohexane-1-carboxamide and 0.328 grams (1.71 mmols) of 3-(((ethylimino)methylene)amino)-N,N-dimethylpropan-1-amine hydrochloride (EDC-HCl). A 25 mL, round-bottom flask (Flask B) was charged with 0.517 grams (4.23 mmols) of 4-dimethylaminopyridine, 0.247 grams (1.41 mmols) of (tert-butoxycarbonyl) glycine, and 10 mL of methylene chloride. The contents of flask B was added to the contents of flask A while magnetically mixing at 250 r.p.m. Flask A was closed with a rubber septum and the head space was purged with dry nitrogen for 4 minutes. The reaction was allowed to continue to mix at 250 r.p.m. and 20-25° C. for 24 hours. Following the reaction period the reaction mixture and 50 mL of anhydrous diethyl ether were added to a 250 mL separatory funnel. The organic layer was extracted with three 20 mL aliquots of 1.0 N HCl, two 20 mL aliquots of saturated sodium bicarbonate, one 20 mL aliquot of distilled water and one 20 mL aliquot of saturated sodium chloride solution. The extracted organic layer was dried over anhydrous sodium sulfate overnight, filtered through Whatman #4 filter to remove any particulates and then the solvent was removed in vacuo to yield 0.30 grams of the amide ester (S)-2-((1R,2S,5R)-2-isopropyl-5-methylcyclohexane-1-carboxamido)-1-phenylethyl (tert-butoxycarbonyl) glycinate. MS(ESI) m/z 461 (MH⁺).

### Example 19 and Example 20

### Syntheses of the isomeric materials (R)-2-((1R,2S,5R)-2-isopropyl-5-methylcyclohexane-1-carboxamido)-1-phenylethyl (tert-butoxycarbonyl)glycinate and (R/S)-2-((1R,2S,5R)-2-isopropyl-5-methylcyclohexane-1-carboxamido)-1-phenylethyl (tert-butoxycarbonyl)glycinate.

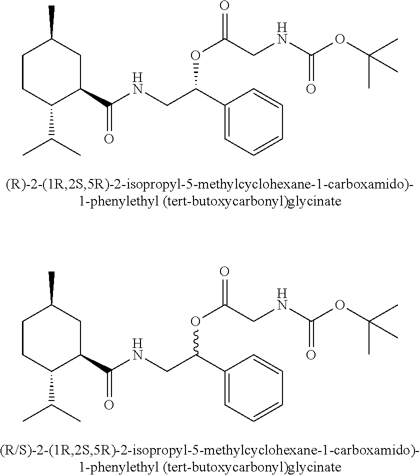 [SMILES: CC(C)[C@@H]1CC[C@@H](C)C[C@H]1C(=O)NC[C@H](OC(=O)CNC(=O)OC(C)(C)C)c1ccccc1 ; CC(C)[C@@H]1CC[C@@H](C)C[C@H]1C(=O)NCC(OC(=O)CNC(=O)OC(C)(C)C)c1ccccc1]

These materials were prepared in the same manner as EXAMPLE 18 starting from (1R,2S,5R)-N-((R)-2-hydroxy-2-phenylethyl)-2-isopropyl-5-methylcyclohexane-1-carboxamide and (1R,2S,5R)-N-((R/S)-2-hydroxy-2-phenylethyl)-2-isopropyl-5-methylcyclohexane-1-carboxamide, respectively.

### Example 21

### Synthesis of (S)-2-((1R,2S,5R)-2-isopropyl-5-methylcyclohexane-1-carboxamide)-1-phenylethyl glycinate hydrochloride

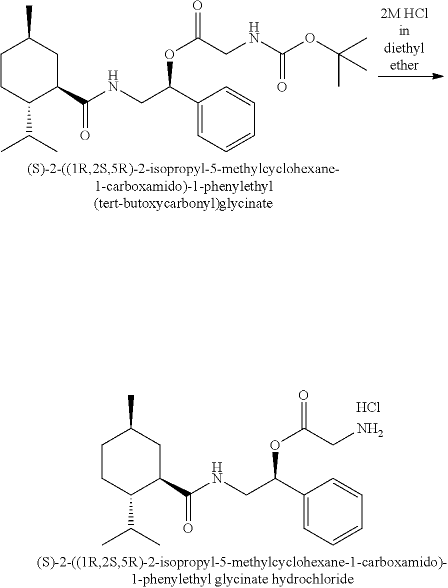

A 100 mL, round-bottom flask (Flask A) was charged with 0.175 grams (0.38 mmols) of the starting (S)-2-((1R,2S ,5R)-2-isopropyl-5-methylcyclohexane-1-carboxamido)-1-phenylethyl (tert-butoxycarbonyl) glycinate and 50 mL of 2.0 M HCl in diethyl ether while magnetically mixing at 250 r.p.m. Flask A was closed with a rubber septum and connected to an oil bubbler. The reactor contents were allowed to mix at 250 r.p.m. and 20-25° C. for 6.5 hours. Following the reaction period the solvent and hydrogen chloride volatiles were removed in vacuo to yield 129 milligrams of the title compound (S)-2-((1R,2S ,5R)-2-isopropyl-5-methylcyclohexane-1-carboxamide)-1-phenylethyl glycinate hydrochloride; MS (ESI) m/z 361 (MH⁺).

### Example 22 and Example 23

### Syntheses of the isomeric materials (R)-2-((1R,2S,5R)-2-isopropyl-5-methylcyclohexane-1-carboxamido)-1-phenylethyl glycinate hydrochloride and (R/S)-2-((1R,2S,5R)-2-isopropyl-5-methylcyclohexane-1-carboxamido)-1-phenylethyl glycinate hydrochloride

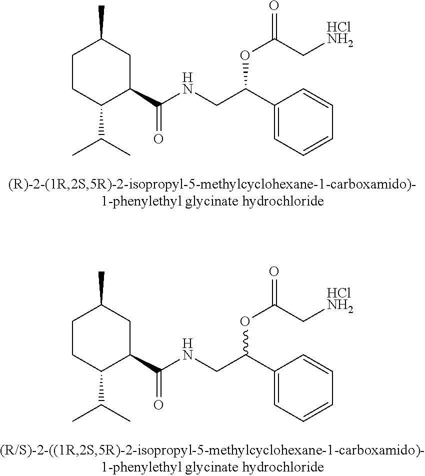

These materials were prepared in the same manner as EXAMPLE 21 starting from (R)-2-((1R,2S,5R)-2-isopropyl-5-methylcyclohexane-1-carboxamido)-1-phenylethyl (tert-butoxycarbonyl) glycinate and (R/S)-2-((1R,2S ,5R)-2-isopropyl-5-methylcyclohexane-1-carboxamido)-1-phenylethyl (tert-butoxycarbonyl) glycinate, respectively.

### Example 24

### Synthesis of (1R,2S,5R)-2-isopropyl-5-methyl-N-(2-oxo-2-phenylethyl)cyclohexane-1-carboxamide

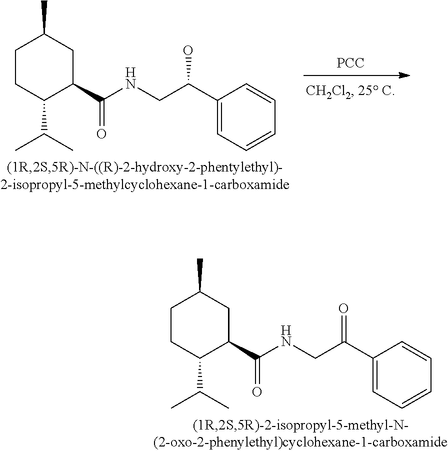 [SMILES: CC(C)[C@@H]1CC[C@@H](C)C[C@H]1C(=O)NC[C@H](O)c1ccccc1 ; "PCC" ; CC(C)[C@@H]1CC[C@@H](C)C[C@H]1C(=O)NCC(=O)c1ccccc1]

A 50 mL, round-bottom flask (Flask A) was charged with 0.499 grams (1.64 mmols) of the starting (1R,2S ,5R)-N-((R)-2-hydroxy-2-phenylethyl)-2-isopropyl-5-methylcyclohexane-1-carboxamide, and 40 mL of anhydrous methylene chloride. A separate 100 mL, round-bottom flask (Flask B) was charged with 0.560 grams (2.60 mmols) of pyridinium chlorochromate (PCC) and 20 mL of anhydrous methylene chloride. The reaction flask B was immersed in an ice bath. The solution in flask A was added to a 60 mL pressure-equalizing addition funnel which was connected to the reaction flask B. The solution of starting alcohol in the addition funnel was added slowly to the dispersion of PCC in the reaction flask B while mixing at 500 r.p.m. over eighteen (18) minutes. The reactor contents were allowed to continue to mix overnight at 250 r.p.m. while allowing the ice bath to melt. Following the reaction period the reaction mixture was added to a 250 mL separatory funnel and extracted with one 50 mL aliquot of 1.0 N HCl. The separation was poor and both layers contained fine, suspended, red/orange precipitate. The two phase system was filtered through a Whatman 934AH filter and the recovered liquid mixture was extracted with three 50 mL aliquots of 1.0 N HCl. The recovered organic layer was transferred to a separate container and shaken with 25 grams of silica gel 60 and the solids allowed to settle. The resulting clear and colorless organic solution was filtered through Whatman #4 filter and then extracted with two, 50 mL aliquots of 1.0 N NaOH and two, 50 mL aliquots of saturated sodium chloride. The extracted organic layer was dried over anhydrous sodium sulfate overnight and filtered through Whatman #4 filter to remove any particulate. The solvent was removed in vacuo to yield 170 milligrams of the title compound (1R,2S ,5R)-2-isopropyl-5-methyl-N-(2-oxo-2-phenylethyl)cyclohexane-1-carboxamide MS(ESI) m/z 302 (MH⁺).

### Example 25

### Synthesis of (1R,2S,5R)-N-((S)-2-amino-4-methylpentyl)-2-isopropyl-5-methylcyclohexane-1-carboxamide.

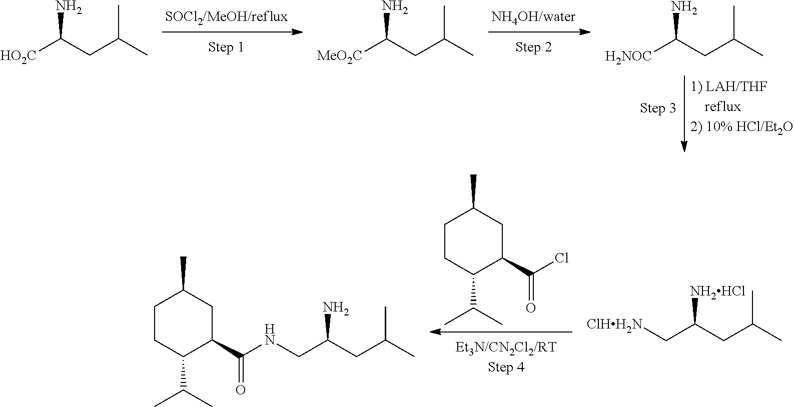

Step 1. In a 1L round-bottomed flask equipped with a reflux condenser capped with an addition funnel was added 50 g of (L)-Leucine (CAS# 61-90-5, 0.367 mol) and 500 mL MeOH. The solution was cooled to 0° C. with an ice-water bath and thionyl chloride (CAS# 7719-09-7, 65 mL) was added dropwise over 30 minutes. The reaction was warmed to room temperature and the addition funnel was removed. The reaction was then refluxed for approximately 24 h and then cooled to room temperature. The solvents were stripped off on a rotary evaporator (Buchi Rotovapor R-124, BÜCHI Labortechnik AG, Switzerland, 5-10 mm Hg) to provide an off-white solid. The solid was dissolved in CH₂Cl₂ (1 L) and washed 3×200 mL sat NaHCO₃ solution until the aqueous layer was basic (pH7-8) by pH paper. The layers were separated, the organic layer was dried (Na₂SO₄), and then the drying agent was filtered off (Buchner funnel). The mother liquor was concentrated under vacuum (5-10 mm Hg) using a rotary evaporator to provide a brownish oil of (L)-Methyl Leucinate; Yield: 18.1 grams

Step 2. Into a 250 mL 24/40 joint single neck round bottomed flask equipped with a stir bar under nitrogen sparge was added a solution of 8.1 grams of (L)-Methyl Leucinate (8.1 g, .056 mol) in 10 mL of MeOH. Aqueous NH₄OH (28-30%, 40 mL) was then added dropwise over approximately 20 minutes. The reaction was stirred 72 h under N₂ atmosphere, and then concentrated under vacuum (˜5-10 mm Hg) on a rotovap (Buchi Rotovapor R-124, BÜCHI Labortechnik AG, Switzerland). The residue was dissolved in water (250 mL) and extracted with CH₂Cl₂ (5×500 mL) using a separatory funnel. The organic layers were combined into a 4L Erlenmeyer flask, then dried (anhydrous Na₂SO₄), filtered to remove drying agent, and concentrated via rotovap under vacuum (5-10 mm Hg) to provide 5 grams of (S)-2-amino-4-methylpentanamide as a white solid.

Step 3. In a 1000 mL 24/40 joint three-neck round-bottom flask equipped with a reflux condenser, a stir bar for stirring and a nitrogen inlet port was added (S)-2-amino-4-methylpentanamide (7 g., 0.0463 mol). The reaction was sparged with N₂, then 300 mL anhydrous tetrahydrofuran (THF) was added via cannula. The reaction was stirred 10 minutes to dissolve the amide, and then solid lithium aluminum hydride (8 g., 95% powder, 0.40 mol) was added portion wise in 6 portions (1-1.5 g each) with rapid stirring over approximately 30 minutes. The reaction was then refluxed using a heating mantle until the starting material was consumed (4-6 hrs) as determined by thin layer chromatography (15% MeOH/CH₂Cl₂ as eluent). The reaction was then cooled to RT by removal of the heating mantle and waiting for one hr. The reaction was placed in an ice-water bath for 30 minutes as stirring was continued. 10% aqueous NaOH solution (50 mL) was added via additional funnel slowly over 1 hr to quench excess lithium aluminum hydride. After the reaction was quenched, as determined by the ceasing of gas evolution, Filtrol 150 clay (20 g) and Celite 545 (20 g) were added portion wise using a spatula, and the mixture was stirred using a stir bar for 2 hr. The mixture was then vacuum filtered (5-10 mm Hg) using a Buchner funnel through a pad of Celite 545 and the pad was washed thoroughly with THF (4×250 mL, 1L total volume). The filtrate was collected in a 2L filter flask and concentrated on a rotovap (Buchi Rotovapor R-124, 5-10 mm Hg) to provide a yellow oil. The oil was dissolved at RT in THF (150 mL) and 2 M HCl/Et₂O (50 mL) was added drop wise with an addition funnel over 15 minutes. The reaction was stirred using a stir bar overnight at RT and the product was filtered off under vacuum (5-10 mm Hg) using a Buchner funnel to provide (S)-4-methylpentane-1,2-diamine dihydrochloride as a white solid. 7 g. MS (ESI): 116.

Step 4. In a 500 mL 2-neck round bottom flask equipped with stir bar, N₂ inlet for inert gas and an additional funnel was dissolved (1R,2S,5R)-2-isopropyl-5-methylcyclohexane-1-carbonyl chloride (5.0 g, 24.75 mmol) in anhydrous CH₂Cl₂ (350 mL) under N₂ atmosphere. The reaction was cooled to 0° C. with an ice-water bath and solid (S)-4-methylpentane-1,2-diamine dihydrochloride salt was added in a single portion via spatula (5.2 g, 27.96 mmol) while Et₃N (20 mL) simultaneously was added via the addition funnel. The reaction was maintained at 0° C. (ice bath) for 1 hr, then the bath was removed and the reaction was warmed to RT and stirred with a stir bar for 72 h. The reaction was quenched by pouring into a 1000 mL separatory funnel containing saturated NaCl solution (300 mL). The layers were separated, and the aqueous layer was extracted with CH₂Cl₂ (3×100 mL). The organic layers were combined in a 1L Erlenmeyer flask and then dried over anhydrous Na₂SO₄ for 1 hr. The drying agent was removed by gravity filtration and the filtrate was concentrated under vacuum (Buchi Rotovapor R-124, 5-10 mm Hg) to provide a yellow foam. The foam was chromatographed (SiO₂, 10% MeOH-CH₂Cl₂) to provide (1R,2S,5R)-N-((S)-2-amino-4-methylpentyl)-2-isopropyl-5-methylcyclohexane-1-carboxamide as a yellow oil. 2.70 g; MS: (ESI) 283 (M⁺H⁺).

### Example 26

### Synthesis of (1R,2S,5R)-N-((S)-2-(((R)-3-aminobut-1-en-2-yl)amino)-4-methylpentyl)-2-isopropyl-5-methylcyclohexane-1-carboxamide hydrochloride.

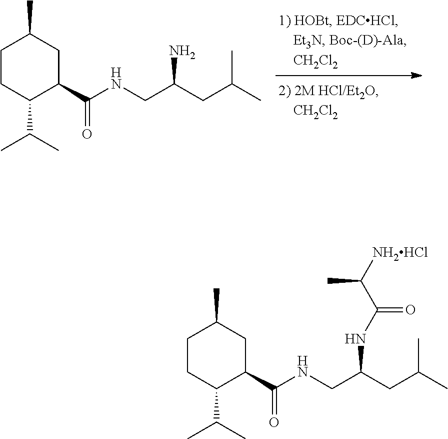

A 250 mL 3-neck round bottom flask equipped with a condenser with an outlet to a Firestone valve (positive nitrogen pressure) and a magnetic stir bar was charged with Boc-D-Ala (0.210 g, 1.10 mmol), HOBt (0.150 g, 1.10 mmol), EDC-HCl (0.211 g, 1.10 mmol), and 85 mL anhydrous methylene chloride. The solution was stirred at room temperature under nitrogen and triethylamine (310 μL, 2.20 mmol) was added. To the heterogeneous mixture was added (1R,2S,5R)-N-((S)-2-amino-2-phenylethyl)-2-isopropyl-5-methylcyclohexane-1-carboxamide (0.282 g, 1 mmol) dissolved in 40 mL of CH₂Cl₂. The heterogeneous mixture was stirred at room temperature under a nitrogen atmosphere for 3 hours. The reaction mixture was transferred to a 1L separatory funnel containing methylene chloride (100 mL) and water (100 mL). The aqueous layer was separated and extracted again with 2×80 mL of methylene chloride. The combined organic phases were washed with 1N HCl solution (2×50 mL), H₂O (1×50 mL), saturated sodium bicarbonate solution (3×50 mL), and brine (1×50 mL). The solution was dried over anhydrous sodium sulfate, filtered, and concentrated under vacuum at 38° C. to give N-Boc protected carboxamide.

The carboxamide was then dissolved in CH₂Cl₂ (20 mL) and 2M HCl/Et₂O solution (5 mL) was added dropwise over 15 minutes. The reaction was stirred 24 h at room temperature and then the solvent was removed via rotary evaporator (Buchi Rotovapor R-124, BÜCHI Labortechnik AG, Switzerland) under reduced pressure (5-10 mm Hg). The residue was triturated with Et₂O (3×100 mL) and the product dried under reduced vacuum (5-10 mm Hg) for 24 h to provide (1R,2S,5R)-N-((S)-2-(((R)-3-aminobut-1-en-2-yl)amino)-4-methylpentyl)-2-isopropyl-5-methylcyclohexane-1-carboxamide hydrochloride; 263 mg. MS: (ESI) 354.

By using a procedure similar to that in EXAMPLE 26 and by modifying the amino acid capping group, the following compounds can be synthesized by those skilled in the art:
(1R,2S,5R)-N-((S)-2-(((S)-3-aminobut-1-en-2-yeamino)-4-methylpentyl)-2-isopropyl-5-methylcyclohexane-1-carboxamide hydrochloride.
(1R,2S,5R)-N-((S)-2-((3-aminoprop-1-en-2-yl)amino)-4-methylpentyl)-2-isopropyl-5-methylcyclohexane-1-carboxamide hydrochloride.
(1R,2S,5R)-N-((S)-2-((S)-2-amino-4-(methylthio)butanamido)-4-methylpentyl)-2-isopropyl-5-methylcyclohexane-1-carboxamide hydrochloride.

### Example 27

### Synthesis of (1R,2S,5R)-N-((S)-2-amino-3-phenylpropyl)-2-isopropyl-5-methylcyclohexane-1-carboxamide

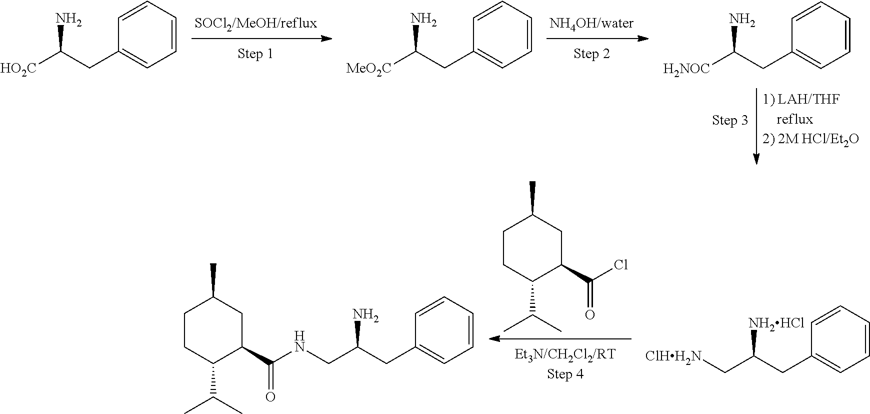

Step 1. In a 1L round-bottomed flask equipped with a reflux condenser capped with an addition funnel was added 50 g (S)-Phenylalanine (0.302 mol) and 500 mL MeOH. The solution was cooled to 0° C. with an ice-water bath and thionyl chloride (CAS# 7719-09-7, 75 mL) was added dropwise over 30 minutes. The reaction was warmed to RT and the addition funnel was removed. The reaction was then refluxed for approximately 24 h, and then cooled to RT. The solvents were stripped off on a rotary evaporator (Buchi Rotovapor R-124, BÜCHI Labortechnik AG, Switzerland, 5-10 mm Hg) to provide an off-white solid. The solid was dissolved in CH₂Cl₂ (1 L) and washed 3×200 mL sat NaHCO₃ solution until the aqueous layer was basic (pH7-8) by pH paper. The layers were separated and the organic layer was dried (Na₂SO₄) and then the drying agent was filtered off (Buchner funnel). The mother liquor was concentrated under vacuum (5-10 mm Hg) using a rotary evaporator to provide a brownish oil; Yield: 45.7 grams.

Step 2. Into a 1000 mL 24/40 joint single neck round bottomed flask equipped with a stir bar under nitrogen sparge was added a solution comprising 45.7 grams of (S)-Phenylalanine methyl ester (0.255 mol) in 200 mL MeOH. Aqueous NH₄OH (28-30%, 200 mL) was then added dropwise over approximately 20 minutes. The reaction was stirred 72 h under N₂ atmosphere, and then concentrated under vacuum (˜5-10 mm Hg) on a rotovap (Buchi Rotovapor R-124, BÜCHI Labortechnik AG, Switzerland). The residue was dissolved in water (250 mL) at RT and extracted with CH₂Cl₂ (5×500 mL) using a separatory funnel. The organic layers were combined into a 4L Erlenmeyer flask, dried (anhydrous Na₂SO₄), filtered to remove drying agent and concentrated via rotovap under vacuum (5-10 mm Hg) to provide a white solid; 48 grams.

Step 3. In a 1000 mL 24/40 joint three-neck round-bottom flask equipped with a reflux condenser, a stir bar for stirring and a nitrogen inlet port was added (S)-2-amino-3-phenylpropanamide (25 g, 0.152 mol). The reaction was sparged with N₂, then 300 mL anhydrous tetrahydrofuran (THF) was added via cannula. The reaction was stirred 10 minutes to dissolve the amide, and then solid lithium aluminum hydride (18 g, 95% powder, 0.45 mol) was added portion wise in 6×3 g portions with rapid stirring over approximately 30 minutes. The reaction was then refluxed using a heating mantle until the starting material was consumed (4 hrs) as determined by thin layer chromatography (15% MeOH/CH2Cl₂ as eluent). The reaction was then cooled to RT by removal of the heating mantle and waiting for one hr. The reaction was placed in an ice-water bath for 30 minutes as stirring was continued. 10% aqueous NaOH solution (50 mL) was added slowly via additional funnel slowly over 1 hr to quench excess lithium aluminum hydride. After the reaction was quenched, as determined by the ceasing of gas evolution, Filtrol 150 clay (20 g) and Celite 545 (50 g) were added portion wise using a spatula and the mixture was stirred using a stir bar for 2 hr. The mixture was then vacuum filtered (5-10 mm Hg) using a Buchner funnel through a pad of Celite 545 and the pad was washed thoroughly with THF (4×250 mL, 1L total volume). The filtrate was collected in a 2L filter flask and concentrated on a rotovap (Buchi Rotovapor R-124, 5-10 mm Hg) to provide a pale green oil. The oil was dissolved at RT in MeOH (150 mL) and 2 M HCl/Et₂O (100 mL) was added drop wise with an addition funnel over 15 minutes. The reaction was stirred using a stir bar overnight and the product was filtered off under vacuum (5-10 mm Hg) using a Buchner funnel to provide 20 g of (S)-3-phenylpropane-1,2-diamine dihydrochloride as an off-white solid.

Step 4. In a 500 mL 2-neck round bottom flask equipped with stir bar, N₂ inlet for inert gas and an additional funnel was dissolved (1R,2S,5R)-2-isopropyl-5-methylcyclohexane-1-carbonyl chloride (5.0 g, 24.75 mmol) in anhydrous CH₂Cl₂ (350 mL) under N₂ atmosphere. The reaction was cooled to 0° C. with an ice-water bath and solid (S)-3-phenylpropane-1,2-diamine dihydrochloride salt was added in a single portion via spatula (4.1 g, 27.22 mmol) while Et₃N (20 mL) simultaneously was added via the addition funnel. The reaction was maintained at 0° C. (ice bath) for 1 hr, then the bath was removed and the reaction was warmed to RT and stirred with a stir bar for 72 h. The reaction was quenched by pouring into a 1000 mL separatory funnel containing saturated NaCl solution (300 mL). The layers were separated, and the aqueous layer was extracted with CH₂Cl₂ (3×100 mL). The organic layers were combined in a 1L Erlenmeyer flask and then dried over anhydrous Na₂SO₄ for 1 hr. The drying agent was removed by gravity filtration and the filtrate was concentrated under vacuum (Buchi Rotovapor R-124, 5-10 mm Hg) to provide a yellow oil. The oil was chromatographed (SiO₂, 10%MeOH-CH₂Cl₂) to provide 3.5 grams of (1R,2S,5R)-N-((S)-2-amino-3-phenylpropyl)-2-isopropyl-5-methylcyclohexane-1-carboxamide as a waxy solid. MS: (ESI) 317 (M+H).

### Example 28

### Synthesis of (1R,2S, 5R)-N-((S)-2-((R)-2-aminopropanamido)-3-phenylpropyl)-2-isopropyl-5-methylcyclohexane-1-carboxamide hydrochloride

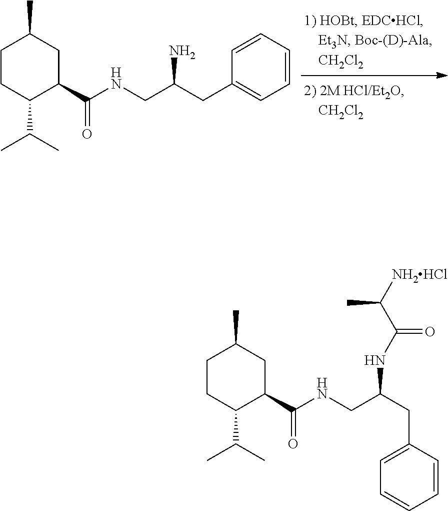

A 250 mL 3-neck round bottom flask equipped with a condenser with an outlet to a Firestone valve (positive nitrogen pressure) and a magnetic stir bar was charged with Boc-D-Ala (0.360 g, 1.75 mmol), HOBt (0.258 g, 1.90 mmol), EDC-HCl (0.364 g, 1.90 mmol), and 100 mL anhydrous methylene chloride. The solution was stirred at room temperature under nitrogen and triethylamine (530 μL, 7.20 mmol) was added. To the heterogeneous mixture was added (1R,2S,5R)-N-((S)-2-amino-3-phenylpropyl)-2-isopropyl-5-methylcyclohexane-1-carboxamide (500 mg, 1.58 mmol) dissolved in 20 mL of CH₂Cl₂. The heterogeneous mixture was stirred under a nitrogen atmosphere for 3 hours. The reaction mixture was transferred to a 1L separatory funnel containing methylene chloride (100 mL) and water (100 mL). The aqueous layer was separated and extracted again with 2×80 mL of methylene chloride. The combined organic phases were washed with 1N HCl solution (2×50 mL), H₂O (1×50 mL), saturated sodium bicarbonate solution (3×50 mL), and brine (1×50 mL). The solution was dried over anhydrous sodium sulfate, filtered, and concentrated under vacuum at 38° C. to give N-Boc protected carboxamide.

The carboxamide was then dissolved in CH₂Cl₂ (20 mL) and 2M HCl/Et₂O solution was added dropwise over 15 minutes (20 mL). The reaction was stirred 24 h and then the solvent was removed via rotary evaporator (Buchi Rotovapor R-124, BÜCHI Labortechnik AG, Switzerland) under reduced pressure (5-10 mm Hg). The residue was triturated with Et₂O (3×100 mL) and the product dried under reduced vacuum (5-10 mm Hg) for 24 h to provide (1R,2S,5R)-N-((S)-2-((R)-2-aminopropanamido)-3-phenylpropyl)-2-isopropyl-5-methylcyclohexane-1-carboxamide hydrochloride as a white solid. 574 mg; MS: (ESI) (M-HCl) 390.

By using a procedure similar to that in EXAMPLE 28 and by modifying the amino acid capping group, the following compounds can be synthesized by those skilled in the art:
(1R,2S, 5R)-N-((S)-2-((S)-2-aminopropanamido)-3-phenylpropyl)-2-isopropyl-5-methylcyclohexane-1-carboxamide hydrochloride.
(1R,2S, 5R)-N-((S)-2-(2-aminoacetamido)-3-phenylpropyl)-2-isopropyl-5-methylcyclohexane-1-carboxamide hydrochloride.
(1R,2S,5R)-N-((S)-2-((S)-2-amino-4-(methylthio)butanamido)-3-phenylpropyl)-2-isopropyl-5-methylcyclohexane-1-carboxamide hydrochloride.

### Example 29 Synthesis of (1R,2S, 5R)-N-((S)-2-amino-2-(chlorophenyl)ethyl)-2-isopropyl-5-methylcyclohexane-1-carboxamide.

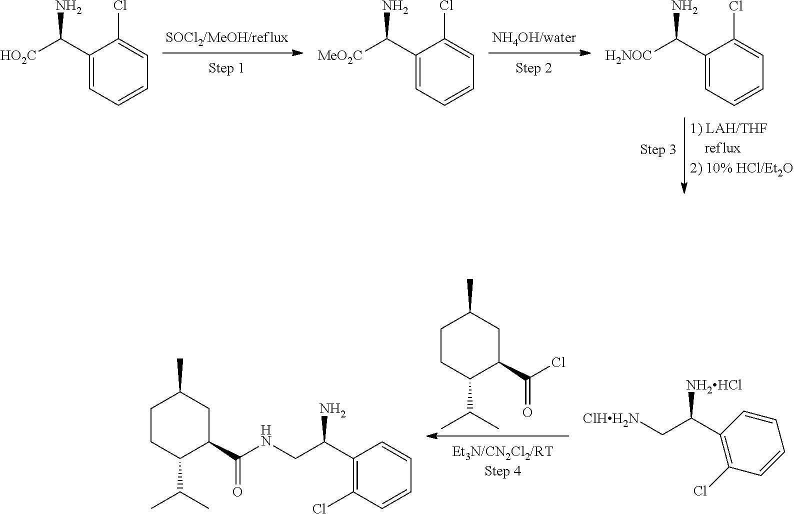

Step 1. In a 500 mL round-bottomed flask equipped with a reflux condenser capped with an addition funnel was added 5.0 g (S)-2-chlorophenylglycine (CAS# 141315-50-6, 27.02 mol) and 200 mL MeOH. The solution was cooled to 0° C. with an ice-water bath and thionyl chloride (CAS# 7719-09-7, 25 mL) was added dropwise over 60 minutes. The reaction was warmed to RT for 24 hr. The solvents were stripped off on a rotary evaporator (Buchi Rotovapor R-124, BÜCHI Labortechnik AG, Switzerland, 5-10 mm Hg) to provide an off-white solid. The solid was dissolved in CH₂Cl₂ (500 mL) and washed 3×200 mL sat NaHCO₃ solution until the aqueous layer was basic (pH7-8) by pH paper. The layers were separated and the organic layer was dried (Na₂SO₄) and then the drying agent was filtered off (Buchner funnel). The mother liquor was concentrated under vacuum (5-10 mm Hg) using a rotary evaporator to provide the methyl ester as a white solid. 5 grams.

Step 2. Into a 500 mL 24/40 joint single neck round bottomed flask equipped with a stir bar under nitrogen sparge was added a solution 5 grams of (S)-chlorophenylglycine methyl ester (0.025 mol) in 200 mL MeOH. Aqueous NH₄OH (28-30% in water, 150 mL) was then added dropwise over approximately 20 minutes. The reaction was stirred 72 h under N₂ atmosphere, and then concentrated under vacuum (˜5-10 mm Hg) on a rotovap (Buchi Rotovapor R-124, BÜCHI Labortechnik AG, Switzerland). The residue was dissolved in water (250 mL), and extracted with CH₂Cl₂ (5×500 mL) using a separatory funnel. The organic layers were combined into a 2L Erlenmeyer flask, then dried (anhydrous Na₂SO₄), filtered to remove drying agent and concentrated via rotovap under vacuum (5-10 mm Hg) to provide 3 grams of amide as a white solid.

Step 3. In a 300 mL 24/40 joint two-neck round-bottom flask equipped with a reflux condenser, a stir bar for stirring and a nitrogen inlet port was added (S)-2-amino-2-(2-chlorophenyl) acetamide (3 g., 0.016 mol). The reaction was sparged with N₂, then 100 mL anhydrous tetrahydrofuran (THF) was added via cannula. The reaction was stirred 10 minutes to dissolve the amide, and then solid lithium aluminum hydride (1.82 g., 95% powder, 0.047 mol) was added portion wise in 3x 600 milligram portions with rapid stirring over approximately 30 minutes. The reaction was then refluxed using a heating mantle until the starting material was consumed (4 hrs) as determined by thin layer chromatography (15% MeOH/CH₂Cl₂ as eluent). The reaction was then cooled to RT by removal of the heating mantle and waiting for one hr. The reaction was placed in an ice-water bath for 30 minutes, as stirring was continued. 10% aqueous NaOH solution (50 mL) was added slowly via additional funnel slowly over 1 hr to quench excess lithium aluminum hydride. After the reaction was quenched, as determined by the ceasing of gas evolution, Filtrol 150 clay (20 g) and Celite 545 (50 g) were added portion wise using a spatula and the mixture was stirred using a stir bar for 2 hr. The mixture was then vacuum filtered (5-10 mm Hg) using a Buchner funnel through a pad of Celite 545 and the pad was washed thoroughly with THF (4×250 mL, 1L total volume). The filtrate was collected in a 2L filter flask and concentrated on a rotovap (Buchi Rotovapor R-124, 5-10 mm Hg) to provide a pale green oil. The oil was dissolved in MeOH (150 mL) and 2 M HCl/Et₂O (100 mL) was added drop wise with an addition funnel over 15 minutes. The reaction was stirred using a stir bar overnight and the product was filtered off under vacuum (5-10 mm Hg) using a Buchner funnel to provide 2.0 g of (S)-1-(2-chlorophenyl)ethane-1,2-diamine dihydrochloride as an off-white solid; MS: (ESI) M+ (173).

Step 4. In a 500 mL 2-neck round bottom flask equipped with stir bar, N₂ inlet for inert gas and an additional funnel was dissolved (1R,2S ,5R)-2-isopropyl-5-methylcyclohexane-1-carbonyl chloride (1.57 g, 0.0083 mmol) in anhydrous CH₂Cl₂ (100 mL) under N₂ atmosphere. The reaction was cooled to 0° C. with an ice-water bath and solid (S)-1-(2-chlorophenyl)ethane-1,2-diamine dihydrochloride salt was added in a single portion via spatula (2.0 g, 0.0083 mmol) while Et₃N (6 mL) simultaneously was added via the addition funnel. The reaction was maintained at 0° C. (ice bath) for 1 hr, then the bath was removed and the reaction was warmed to RT and stirred with a stir bar for 72 h. The reaction was quenched by pouring into a 500 mL separatory funnel containing saturated NaCl solution (300 mL). The layers were separated, and the aqueous layer was extracted with CH₂Cl₂ (3×100 mL). The organic layers were combined in a 1L Erlenmeyer flask and then dried over anhydrous Na₂SO₄ for 1 hr. The drying agent was removed by gravity filtration and the filtrate was concentrated under vacuum (Buchi Rotovapor R-124, 5-10 mm Hg), to provide crude product. The product was chromatographed (SiO₂, 7% MeOH-CH₂Cl₂) to provide 443 mg of (1R,2S, 5R)-N-((S)-2-amino-2-(chlorophenyl)ethyl)-2-isopropyl-5-methylcyclohexane-1-carboxamide as a white foam; MS: (ESI) M+337.

### Example 30

### Synthesis of (1R,2S,5R)-N-((S)-2-(2-aminoacetamido)-2-(2-chlorophenyl)ethyl)-2-isopropyl-5-methylcyclohexane-1-carboxamide hydrochloride

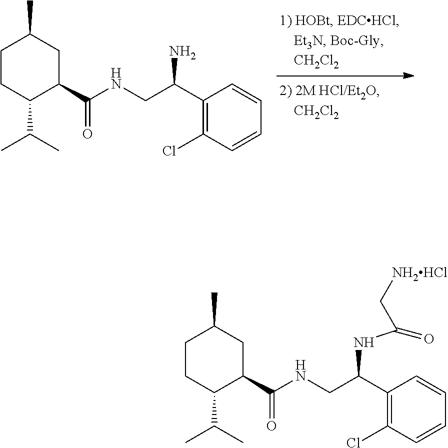

To a dry 50 mL 3-neck round bottom flask equipped with a condenser with an outlet to a Firestone valve (positive nitrogen pressure) and a magnetic stir bar was charged with Boc-Gly (0.0910 g, 0.522 mmol), HOBt (0.070 g, 0.522 mmol), EDC-HCl (0.100 g, 0.522 mmol), and 30 mL anhydrous tetrahydrofuran. The solution was stirred at room temperature under nitrogen and triethylamine (72 μL, 0.055 g, 0.542 mmol) was added. To the heterogeneous mixture was added (1R,2S ,5R)-N-((S)-2-amino-2-(2-chlorophenyl)ethyl)-2-isopropyl-5-methylcyclohexane-1-carboxamide (0.1686 g, 0.501 mmol). The heterogeneous mixture was stirred under a nitrogen atmosphere overnight. The reaction mixture was transferred to a 1 L separatory funnel containing ethyl acetate (70 mL) and distilled water (70 mL). The aqueous layer was separated and extracted with 2×20 mL of ethyl acetate. The combined organic layers were washed with 1N HCl solution (2×50 mL), H₂O (1×50 mL), saturated sodium bicarbonate solution (3×50 mL), and brine (1×50 mL). The solution was dried over anhydrous sodium sulfate, filtered, and concentrated under vacuum at 42° C. to give 0.1936 grams of a white solid. MS (ESI) m/z 394 (MH⁺)

By using a procedure similar to that in EXAMPLE 30 and by modifying the amino acid capping group, the following compounds can be synthesized by those skilled in the art:
(1R,2S,5R)-N-((S)-2-amino-2-(2-chlorophenyl)ethyl)-2-isopropyl-5-methylcyclohexane-1-carboxamide hydrochloride
(1R,2S, 5R)-N-((S)-2-((R)-2-aminopropanamido)-2-(2-chlorophenyl)ethyl)-2-isopropyl-5-methylcyclohexane-1-carboxamide hydrochloride
(1R,2S, 5R)-N-((S)-2-((S)-2-aminopropanamido)-2-(2-chlorophenyl)ethyl)-2-isopropyl-5-methylcyclohexane-1-carboxamide hydrochloride

### Example 31

### (1R,2S,5R)-N-((S)-2-amino-2-cyclohexylethyl)-2-isopropyl-5-methylcyclohexane-1-carboxamide hydrochloride

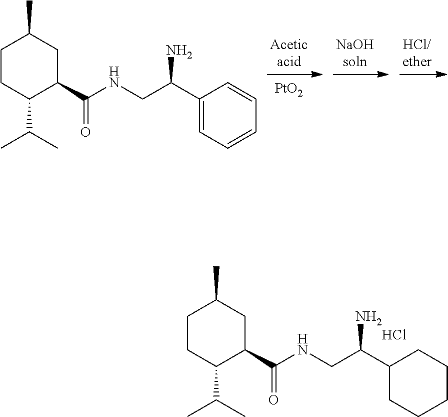

A special order mini-reactor was used for this work that is made by the Parr Instrument Company of Moline, Ill. It is a Parr Model Number NS4703 that is approximately 8 milliliters in internal volume that has wetted materials constructed of T316 stainless steel and PTFE. Agitation was by a magnetically coupled stir bar on a stirring plate. Heated using an oil bath and temperature controlled by an I²R Thermowatch Model Number L7-1100SA/28T.

The Parr reactor (described above) was charged with 58 mg (0.192 mmol) of (1R,2S,5R)-N-((S)-2-amino-2-phenylethyl)-2-isopropyl-5-methylcyclohexane-1-carboxamide. Added 58 mg (0.255 mmol) of platinum oxide and 1.20 ml of glacial acetic acid. Secured the PARR assembly and pressurize to 370 psig hydrogen. Slowly vented to ˜0 psig and repeated 3 times. After the third cycle, the reactor was held at 370 psig hydrogen. The Parr reactor was heated to 50° C. for 60 minutes then allowed to cool to RT; and stirred magnetically for an additional 16 hours. The reactor was slowly vented and the mixture transferred to a syringe filter with chloroform rinses and filtered. The filtrate was washed 2×25 mL 1N NaOH solution, then with 2×25 mL saturated sodium chloride solution. The organic layer was dried over anhydrous sodium sulfate, vacuum filtered and concentrated in vacuo. Recovered 0.0382 g (1R,2S,5R)-N-((S)-2-amino-2-cyclohexylethyl)-2-isopropyl-5-methylcyclohexane-1-carboxamide.

(1R,2S ,5R)-N-((S)-2-amino-2-cyclohexylethyl)-2-isopropyl-5-methylcyclohexane-1-carboxamide (0.0200 g, 0.065 mmol) was dissolved in 1 ml of anhydrous diethyl ether and 1 ml of 2 M hydrogen chloride in diethyl ether and stirred for 30 minutes. The mixture was concentrated in vacuo to yield (1R,2S,5R)-N-n((S)-2-amino-2-cyclohexylethyl)-2-isopropyl-5-methylcyclohexane-1-carboxamide hydrochloride (0.0201 g) as a white powder; MS (ESI) m/z 309 (MH⁺).

### Example 32

### Synthesis of (1R,2S,5R)-N-((S)-2-(2-aminoacetamido)-2-cyclohexylethyl)-2-isopropyl-5-methylcyclohexane-1-carboxamide hydrochloride

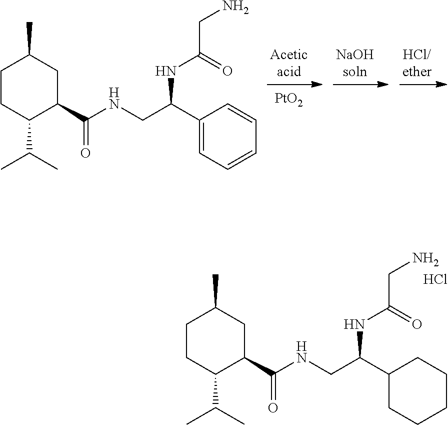

Prepared under the same conditions as (1R,2S,5R)-N-((S)-2-amino-2-cyclohexylethyl)-2-isopropyl-5-methylcyclohexane-1-carboxamide hydrochloride (EXAMPLE 31).

### Example 33

### Synthesis of (1R,2S,5R)-N-((S)-2-((R) -2-aminoacetamido)-2-cyclohexylethyl)-2-isopropyl-5-methylcyclohexane-1-carboxamide hydrochloride

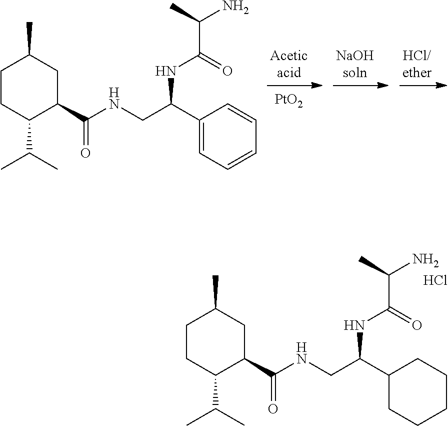

Prepared under the same conditions as (1R,2S,5R)-N-((S)-2-amino-2-cyclohexylethyl)-2-isopropyl-5-methylcyclohexane-1-carboxamide hydrochloride (EXAMPLE 31).

### Example 34

### Synthesis of (1R,2S,5R)-N-((S)-2-((S) -2-aminopropanamido)-2-cyclohexylethyl)-2-isopropyl-5-methylcyclohexane-1-carboxamide hydrochloride

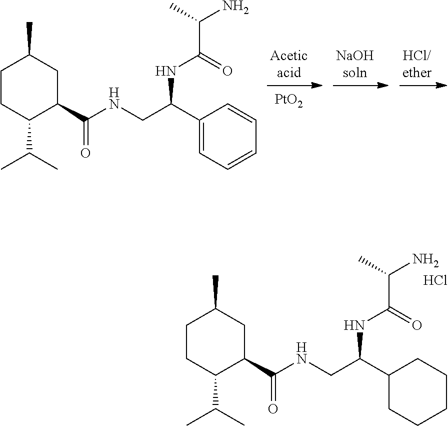

Prepared under the same conditions as (1R,2S,5R)-N-((S)-2-amino-2-cyclohexylethyl)-2-isopropyl-5-methylcyclohexane-1-carboxamide hydrochloride (EXAMPLE 31).

### Example 35

### Synthesis of (1R,2S,5R)-N-((S)-2-amino-2-(2-chlorophenyl)ethyl)-2-isopropyl-5-methylcyclohexane-1-carboxamide hydrochloride

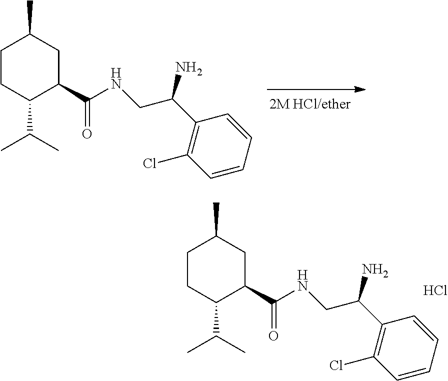

To a scintillation flask containing 0.0261 grams (0.077 mmol) of sample (1R,2S,5R)-N-((S)-2-amino-2-(2-chlorophenyl)ethyl)-2-isopropyl-5-methylcyclohexane-1-carboxamide was added 2 ml of 2 M hydrogen chloride solution in diethyl ether. The mixture was stirred for 30 minutes then concentrated by sweeping with nitrogen followed by placing the vial in a vacuum desiccator for 16 hours at RT. Recovered 0.0287 g of an off-white powder.

### Example 36

### Synthesis of (1R,2S,5R)-N-((S)-2-(2-aminoacetamido)-2-(2-chlorophenyl)ethyl)-2-isopropyl-5-methylcyclohexane-1-carboxamide hydrochloride

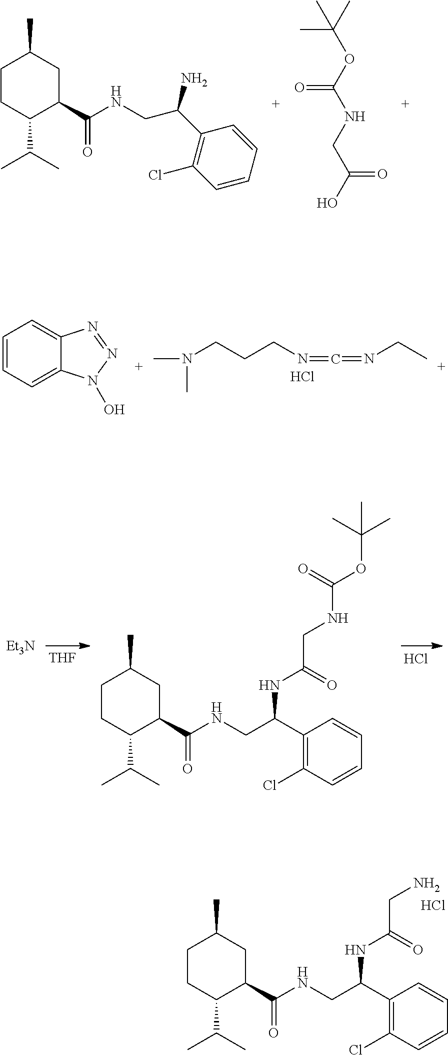

A dry 50 mL 3-neck round bottom flask equipped with a condenser with an outlet to a Firestone valve (positive nitrogen pressure) and a magnetic stir bar was charged with Boc-Gly (0.0910 g, 0.522 mmol), HOBt (0.070 g, 0.522 mmol), EDC-HCl (0.100 g, 0.522 mmol), and 30 mL anhydrous tetrahydrofuran. The solution was stirred under nitrogen and triethylamine (72 μL, 0.055 g, 0.542 mmol) was added. To the heterogeneous mixture was added (1R,2S,5R)-N-((S)-2-amino-2-(2-chlorophenyl)ethyl)-2-isopropyl-5-methylcyclohexane-1-carboxamide (0.1686 g, 0.501 mmol). The heterogeneous mixture was stirred under a nitrogen atmosphere overnight. The reaction mixture was transferred to a 1 L separatory funnel containing ethyl acetate (70 mL) and distilled water (70 mL). The aqueous layer was separated and extracted with 2×20 mL of ethyl acetate. The combined organic layers were washed with1N HCl solution (2×50 mL), H₂O (1×50 mL), saturated sodium bicarbonate solution (3×50 mL), and brine (1×50 mL). The solution was dried over anhydrous sodium sulfate, filtered, and concentrated under vacuum at 42° C. to give 0.1936 grams of a white solid; MS (ESI) m/z 394 (MH⁺).

### Example 37

### Synthesis of (1R,2S,5R)-N-((S)-2-aminopropanamido)-2-(2-chlorophenyl)ethyl)-2-isopropyl-5-methylcyclohexane-1-carboxamide hydrochloride

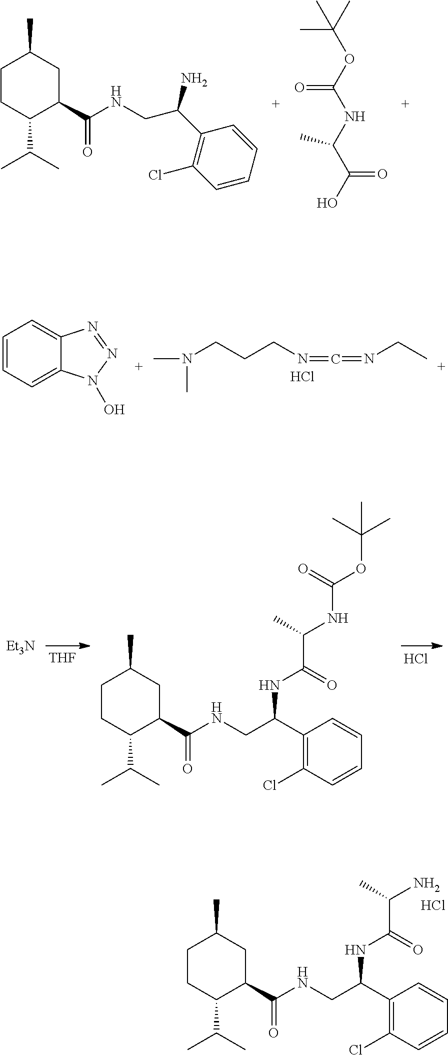

Prepared the same as (1R,2S,5R)-N-((S)-2-(2-aminoacetamido)-2-(2-chlorophenyl)ethyl)-2-isopropyl-5-methylcyclohexane-1-carboxamide hydrochloride (EXAMPLE 36) except Boc-L-Ala was used in place of Boc-Gly.

### Example 38

### Synthesis of (1R,2S,5R)-N-((S)-2-((S)-2-amino-4-methylpentanamido)-2-phenylethyl)-2-isopropyl-5-methylcyclohexane-1-carboxamide hydrochloride

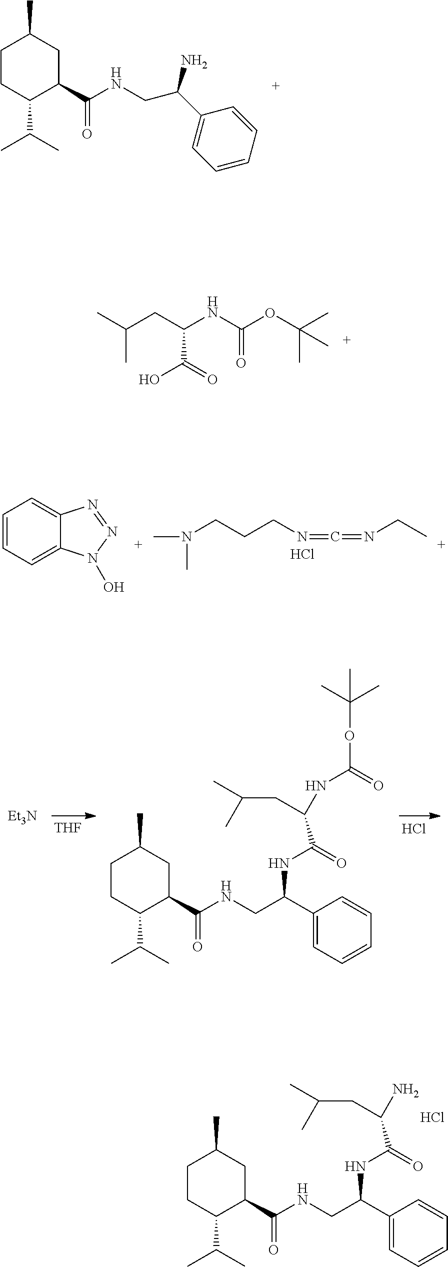

Prepared the same as (1R,2S,5R)-N-((S)-2-(2-aminoacetamido)-2-(2-chlorophenyl)ethyl)-2-isopropyl-5-methylcyclohexane-1-carboxamide hydrochloride (EXAMPLE 36) except Boc-L-Leu was used in place of Boc-Gly.

### Example 39

### Synthesis of (1R,2S,5R)-N-((S)-2-((R)-2-amino-4-methylpentanamido)-2-phenylethyl)-2-isopropyl-5-methylcyclohexane-1-carboxamide hydrochloride

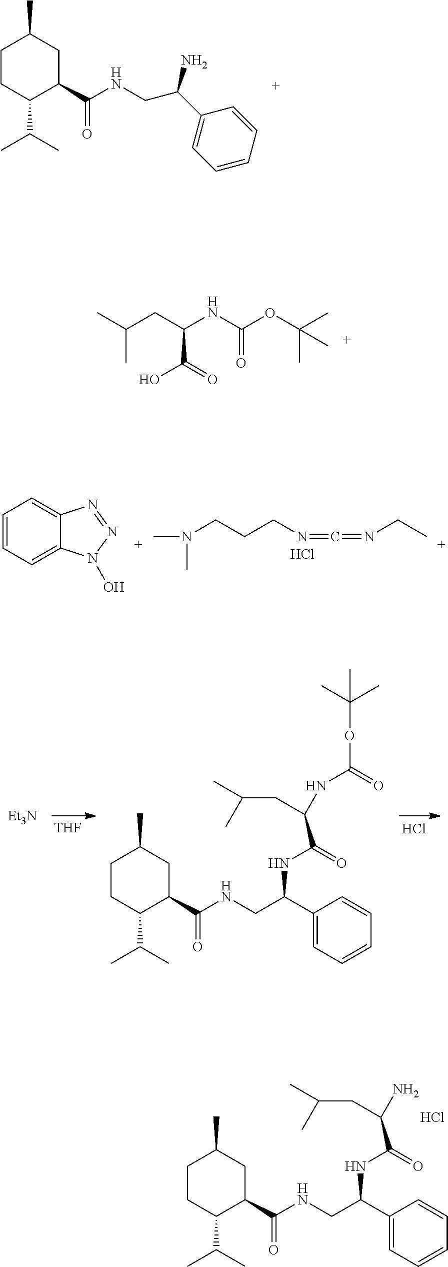

Prepared the same as (1R,2S,5R)-N-((S)-2-(2-aminoacetamido)-2-(2-chlorophenyl)ethyl)-2-isopropyl-5-methylcyclohexane-1-carboxamide hydrochloride (EXAMPLE 36) except Boc-D-Leu was used in place of Boc-Gly.

### Example 40

### Synthesis of (1R,2S,5R)-N-((S)-2-((S)-2-amino-3-(1H-indol-3-yl)propanamido)-2-phenylethyl)-2-isopropyl-5-methylcyclohexane-1-carboxamide hydrochloride

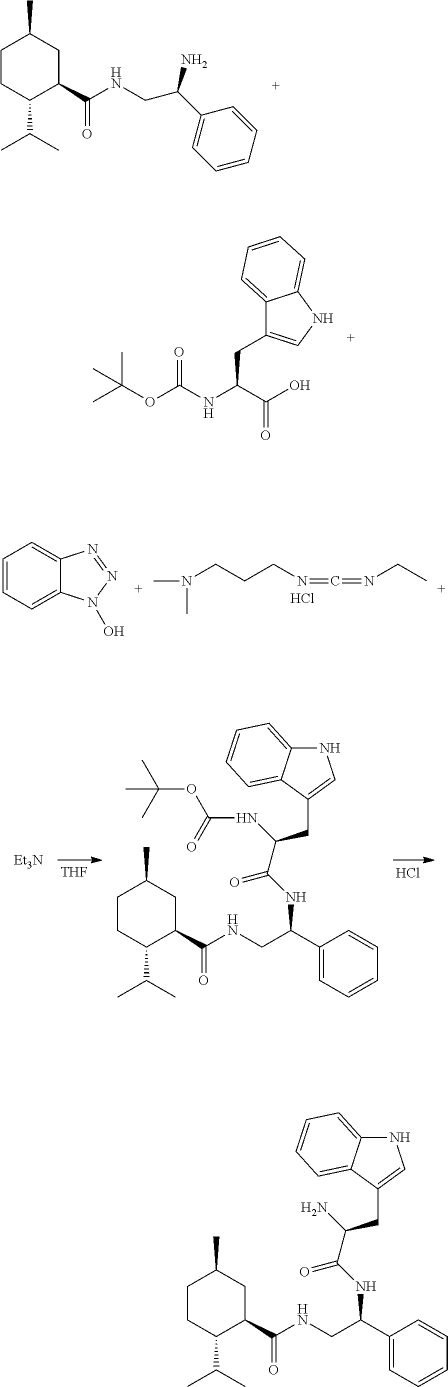

Prepared the same as (1R,2S,5R)-N-((S)-2-(2-aminoacetamido)-2-(2-chlorophenyl)ethyl)-2-isopropyl-5-methylcyclohexane-1-carboxamide hydrochloride (EXAMPLE 36) except Boc-Trp was used in place of Boc-Gly.

### Example 41

### Synthesis of (1R,2S,5R)-N-((S)-2-((S)-2-amino-3-phenylpropanamido)-2-phenylethyl)-2-isopropyl-5-methylcyclohexane-1-carboxamide hydrochloride

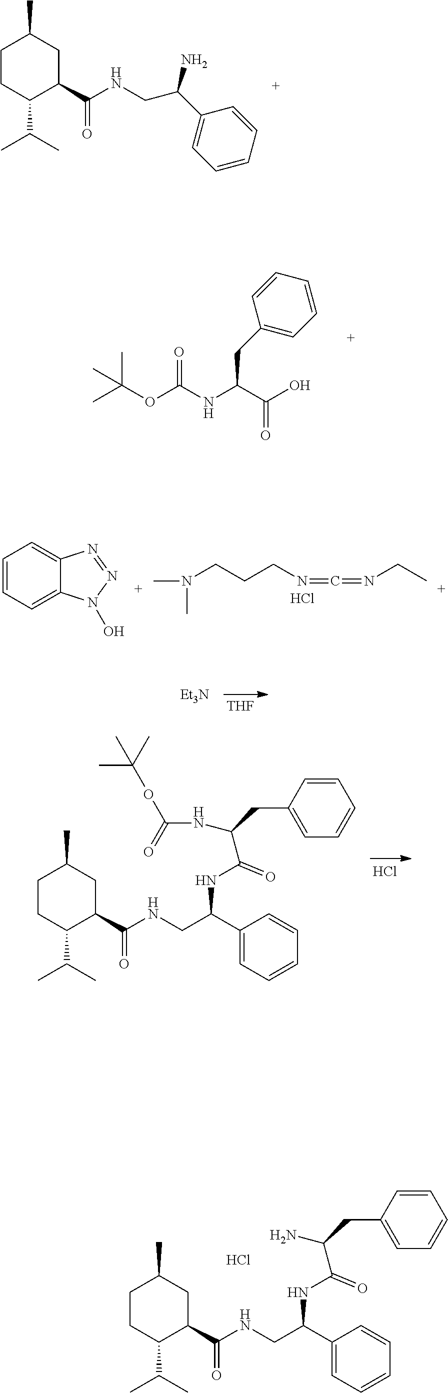

Prepared the same as (1R,2S,5R)-N-((S)-2-(2-aminoacetamido)-2-(2-chlorophenyl)ethyl)-2-isopropyl-5-methylcyclohexane-1-carboxamide hydrochloride (EXAMPLE 36) except Boc-Phe was used in place of Boc-Gly.

### Example 42

### Synthesis of (1R,2S,5R)-N-((R)-2-((S)-2-amino-3-phenylpropanamido)-2-phenylethyl)-2-isopropyl-5-methylcyclohexane-1-carboxamide hydrochloride

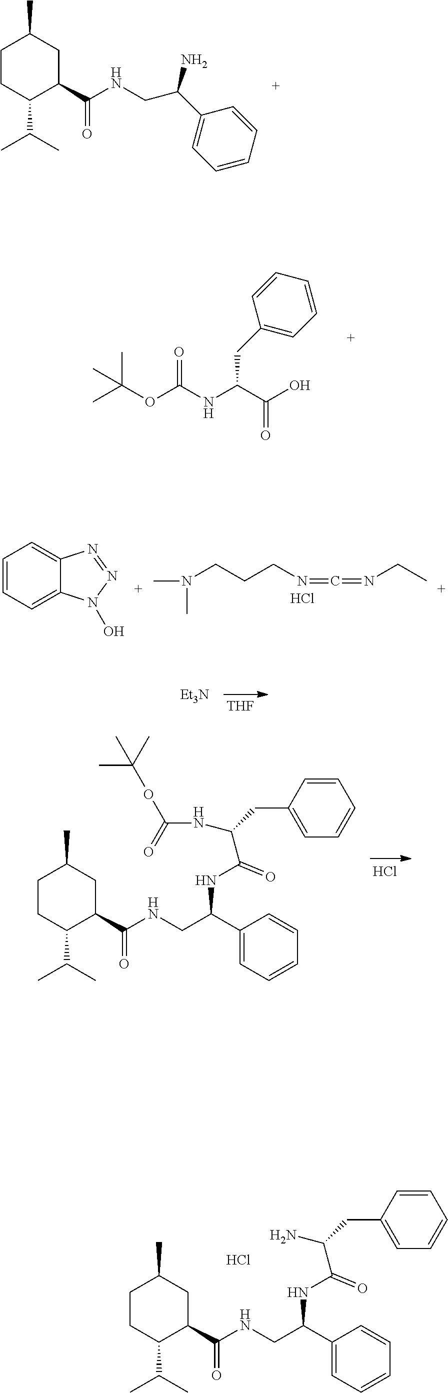

Prepared the same as (1R,2S,5R)-N-((S)-2-(2-aminoacetamido)-2-(2-chlorophenyl)ethyl)-2-isopropyl-5-methylcyclohexane-1-carboxamide hydrochloride (EXAMPLE 36) except Boc-D-Phe was used in place of Boc-Gly.

### Example 43

### Synthesis of (1R,2S,5R)-N-((S)-2-((S)-2,6-diaminohexanamido)-2-phenylethyl)-2-isopropyl-5-methylcyclohexane-1-carboxamide dihydrochloride

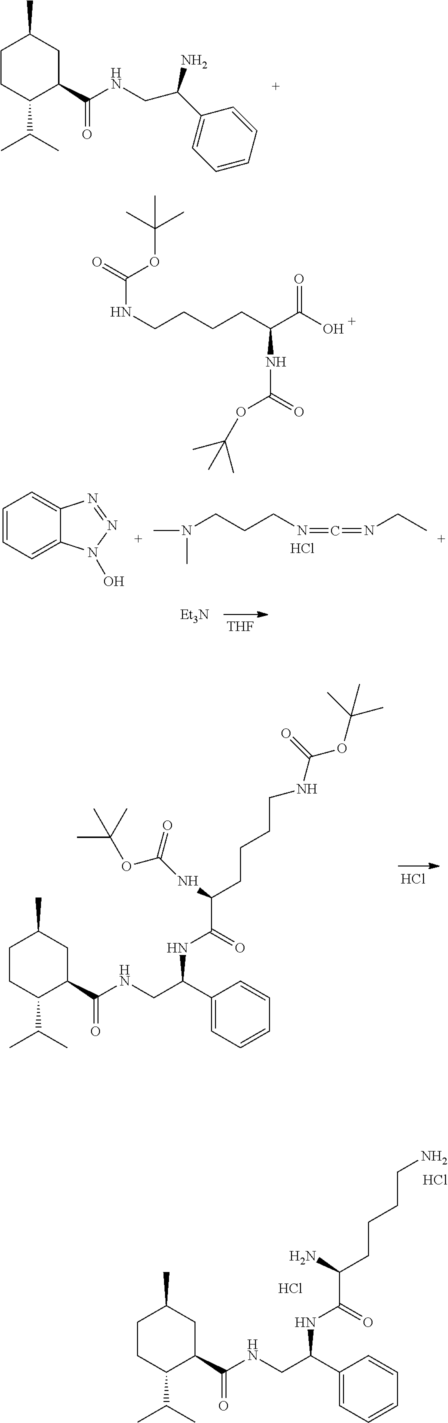

Prepared the same as (1R,2S,5R)-N-((S)-2-(2-aminoacetamido)-2-(2-chlorophenyl)ethyl)-2-isopropyl-5-methylcyclohexane-1-carboxamide hydrochloride (EXAMPLE 36) except Boc-Lys-Boc was used in place of Boc-Gly.

### Example 44

### Synthesis of (1R,2S,5R)-N-((S)-2-(2-(dimethylamino)acetamido)-2-phenylethyl)-2-isopropyl-5-methylcyclohexane-1-carboxamide

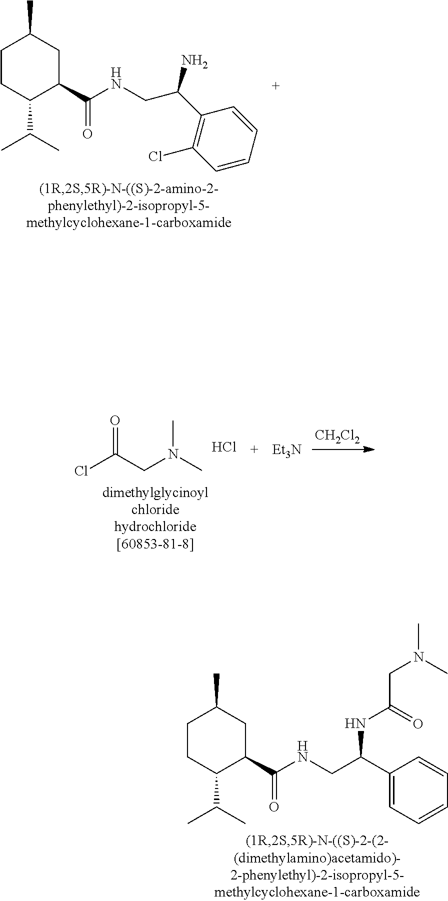

Dimethylaminoacetyl chloride hydrochloride (0.094 g, 0.596 mmol, [60853-81-8], technical grade, “85%”) was weighed out in a 10 mL RB flask with stir bar. A vial was charged with 0.1502 g (0.497 mmol) of (1R,2S ,5R)-N-((S)-2-amino-2-phenylethyl)-2-isopropyl-5-methylcyclohexane-1-carboxamide dissolved in 2 mL of CH₂Cl₂ along with 0.20 mL (1.44 mmol) of Et₃N. The contents of the vial were transferred into the acid chloride suspension with a pipette. The suspension quickly became a homogenous amber solution (acid chloride was brown). The vial was rinsed with an additional 1 mL of CH₂Cl₂ and added to the reaction flask. The flask was capped and stirred and the presence of starting material and product was occasionally monitored by LC/MS analysis. After 6 h an additional 0.055 g (0.348 mmol) of the acid chloride and 0.20 mL (1.44 mmol) of Et₃N were added and stirring at RT was continued. After a total of 24 h the reaction mixture was cloudy with precipitate, and an additional 0.104 g (0.658 mmol) of acid chloride was added. The mixture was stirred an additional 5.5 h. Then 3 mL of 1 M NaOH was added and the mixture was stirred vigorously for 40 min before the mixture was diluted with 10 mL of Et₂O, extracted successively with 10 mL of 1 M NaOH, 10 mL of water, and 10 mL of saturated NaCl solution.

The organic phase was dried over Na₂SO₄, filtered, and solvent was removed via a rotary evaporator under reduced pressure (5-10 mm Hg) at 50° C. to give 0.165 g of the product as an off-white powder. MS (ESI) 385.5 (MH⁺).

### Example 45

### Synthesis of 2-(((S)-2-((1R,2S,5R)-2-isopropyl-5-methylcyclohexane-1-carboxamido)-1-phenylethyl)amino)-N,N,N-trimethyl-2-oxoethan-1-aminium iodide

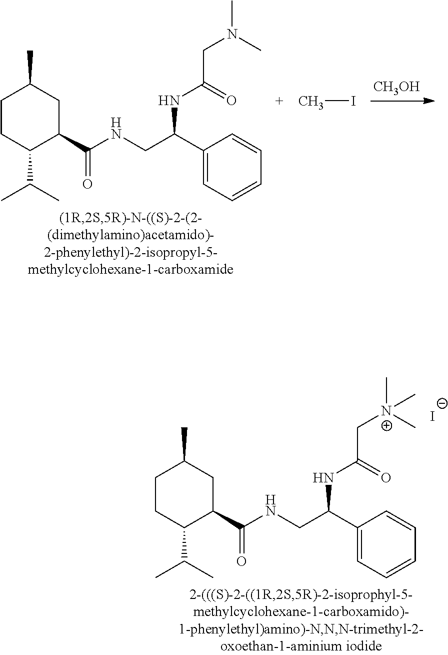

A 0.110 g (0.283 mmol) sample of (1R,2S,5R)-N-((S)-2-(2-(dimethylamino)acetamido)-2-phenylethyl)-2-isopropyl-5-methylcyclohexane-1-carboxamide was dissolved in 1 mL of methanol in a 2 mL vial. After the addition of 50 μL (0.803 mmol) of methyl iodide the solution was shaken and allowed to stand at room temperature for 3.3 h. The volatiles were removed via a rotary evaporator under reduced pressure (5-10 mm Hg) at 40° C. and then placed under vacuum @ 0.08 mm Hg for 44 h to yield the product as a tan solid. MS (ESI) 402.5 (C₂₄H₄₀N₃O₂+=402.31).

### Example 46

### Synthesis of N-((S)-2-((1R,2S,5R)-2-isopropyl-5-methylcyclohexane-1-carboxamido)-1-phenylethyl)picolinamide

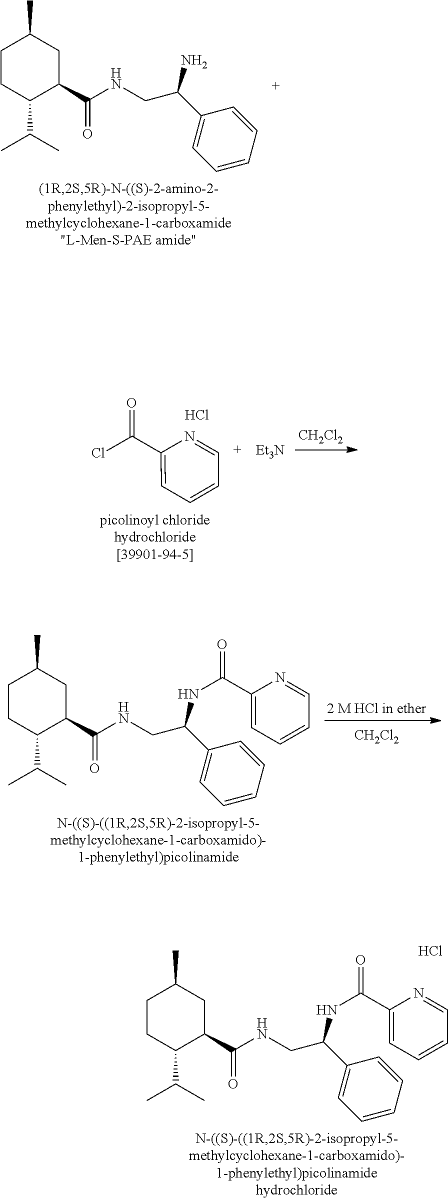

A 100 mL RB flask was charged with 0.1480 g (0.489 mmol) of (1R,2S,5R)-N-((S)-2-amino-2-phenylethyl)-2-isopropyl-5-methylcyclohexane-1-carboxamide and 200 μL (1.44 mmol) of triethylamine dissolved in 5 mL of anhydrous CH₂Cl₂. The mixture was magnetically stirred while picolinoyl chloride hydrochloride [39901-94-5] was added as a solid in one portion. After 24 h 10 mL of 1.0 M NaOH was added and stirred rapidly for 1 h to hydrolyze any of the acid chloride remaining. Then the mixture was rinsed into a separatory funnel with an additional 5 mL of CH₂Cl₂, the organic layer was separated and extracted with another 10 mL of 1.0 M NaOH followed by 10 mL of saturated NaCl solution. The organic phase was dried over Na₂SO₄, filtered, and solvent was removed via a rotary evaporator under reduced pressure (5-10 mm Hg) at 40° C. to give 0.189 g of product as a white solid; MS (ESI) 408 (MH⁺).

### Example 47

### Synthesis of N-((S)-2-((1R,2S,5R)-2-isopropyl-5-methylcyclohexane-1-carboxamido)-1-phenylethyl)picolinamide hydrochloride

N-((S)-2-((1R,2S,5R)-2-isopropyl-5-methylcyclohexane-1-carboxamido)-1-phenylethyl)picolinamide (0.068 g, 0.167 mmol) was placed in a 50 mL conical flask and dissolved in 10 mL of CH₂Cl₂. Upon the addition of 2 mL of 2 M HCl in ether the solution clouds up a little and a sticky residue forms on the walls of the flask. The flask was swirled occasionally for 1 h and then volatiles were removed via a rotary evaporator under reduced pressure (5-10 mm Hg) at 40° C. to give 0.090 g of the hydrochloride as a white solid.

### Example 48

### Synthesis of (1R,2S,5R)-N-(2-aminoethyl)-2-isopropyl-5-methylcyclohexane-1-carboxamide hydrochloride

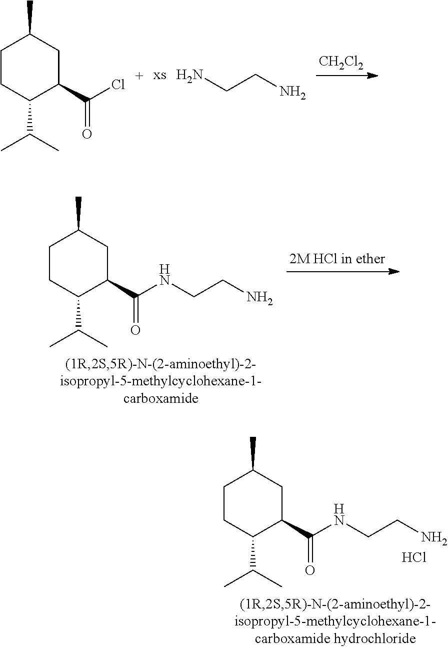

A 25 mL RB flask was charged with 3.0 mL (2.7 g, 44.9 mmol) of ethylene diamine and 1 mL of anhydrous CH₂Cl₂ along with a magnetic stir bar and fitted with a capped 10 mL pressure-equalizing addition funnel containing 0.203 g (1.00 mmol) of (1R,2S,5R)-2-isopropyl-5-methylcyclohexane-1-carbonyl chloride and 2 mL of anhydrous CH₂Cl₂. The flask was chilled in an ice bath, the acid chloride/CH₂Cl₂ mixture was added dropwise over 1 min to the stirring ethylene diamine/CH₂Cl₂ mixture, and the addition funnel was rinsed with 1/2 mL of CH₂Cl₂. After 5 min the ice bath was removed and the reaction was stirred at RT for 110 min. After this time 10 mL of 1 M sodium hydroxide solution was added, the layers were separated, and the aqueous layer was extracted with another 10 mL of CH₂Cl₂. The combined CH₂Cl₂ layers were washed with 5 mL of water, then with 10 mL of saturated NaCl solution, dried over Na₂SO₄, filtered to remove the Na₂SO₄, and then volatiles were removed via a rotary evaporator under reduced pressure (5-10 mm Hg) at 40° C. to give 0.189 g of (1R,2S,5R)-N-(2-aminoethyl)-2-isopropyl-5-methylcyclohexane-1-carboxamide as a sticky residue; MS (ESI) 227 (MH⁺). A portion (0.015 g) of this material in a vial was treated with 1 mL of 2.0 M HCl in diethyl ether to make the hydrochloride salt. After 5 min this volatiles were removed via a rotary evaporator under reduced pressure (5-10 mm Hg) at 40° C. to give (1R,2S,5R)-N-(2-aminoethyl)-2-isopropyl-5-methylcyclohexane-1-carboxamide hydrochloride.

### Example 49

### Synthesis of tert-butyl (2-((2-((1R,2S,5R)-2-isopropyl-5-methylcyclohexane-1-carboxamido)ethyl)amino)-2-oxoethyl)carbamate

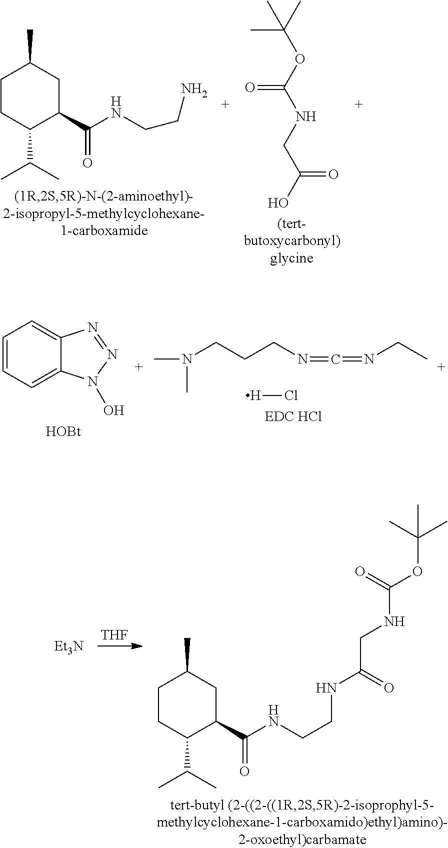

A 2-neck 50 mL RB flask fitted with an vacuum-argon inlet, stopper, and a magnetic stir bar was charged with 0.156 g (0.890 mmol) of (tert-butoxycarbonyl)glycine, 0.123 g (0.910 mmol) of HOBt, and 0.178 g (0.929 mmol) of EDC HCl. Then 5 mL of HPLC grade THF was added through side arm with Ar flowing through, 124 μL (0.891 mmol) of triethylamine was added, and the system was vacuum-argon cycled 5 times and left under an argon atmosphere. A solution of 0.178 g (0.786 mmol) of (1R,2S ,5R)-N-(2-aminoethyl)-2-isopropyl-5-methylcyclohexane-1-carboxamide in 15 mL of HPLC grade THF was added through the side arm with argon flowing through. The system was again cycled 5 times between vacuum and argon, and the cloudy mixture was stirred under an argon atmosphere. After 185 min the reaction mixture was transferred into a separatory funnel along with 25 mL of ethyl acetate and 25 mL of water. The aqueous phase was separated and re-extracted with ethyl acetate (2×20 mL). The organic phase was extracted with 1N HCl (2×10 mL), H₂O (1×10 mL), saturated NaHCO₃ solution (3×10 mL), and saturated NaCl solution (10 mL). This solution was dried over Na₂SO₄ overnight, filtered, and solvent was removed via a rotary evaporator under reduced pressure (5-10 mm Hg). The residue was further vacuum dried at 0.1 mm Hg overnight to provide 0.213 g of the product as an off-white solid residue; MS (ESI) 384 (MH⁺, 100%), 328 (75%), 284 (30%).

### Example 50

### Synthesis of (1R,2S,5R)-N-(2-(2-aminoacetamido)ethyl)-2-isopropyl-5-methylcyclohexane-1-carboxamide hydrochloride

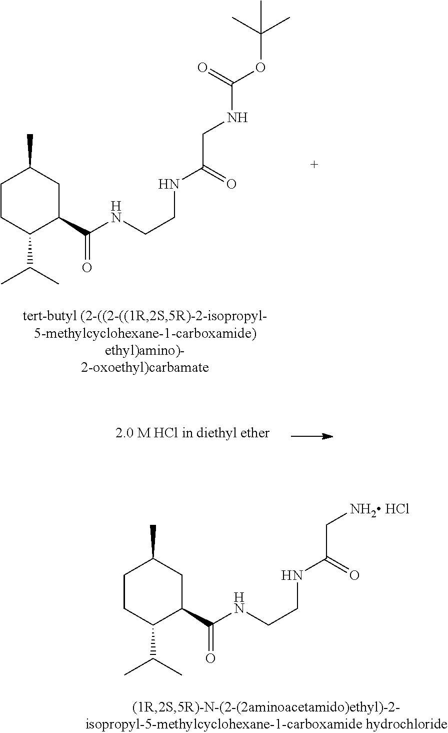

A magnetic stir bar was added to a 100 mL conical flask which contained 0.167 g (0.435 mmol) of tert-butyl (2-((2-((1R,2S ,5R)-2-isopropyl-5-methylcyclohexane-1-carboxamido)ethyl)amino)-2-oxoethyl)carbamate. The addition of 10 mL of 2.0 M HCl/ether (20 mmol) led to a lot of undissolved (softened solid) material on walls of the flask. The addition of 10 mL of CH₂Cl₂ helped put the solution in contact with this material, but undissolved material remained. After stirring for 25 h the stir bar was removed and the volatiles were removed via a rotary evaporator under reduced pressure (5-10 mm Hg) to give 0.148 g of the hydrochloride as a white solid. MS (ESI) 284 (C₁₅H₃₀N₃O₂⁺=284).

### Example 51

### Synthesis of (1R,2S,5R)-N-(5-aminopentyl)-2-isopropyl-5-methylcyclohexane-1-carboxamide hydrochloride

Step 1.

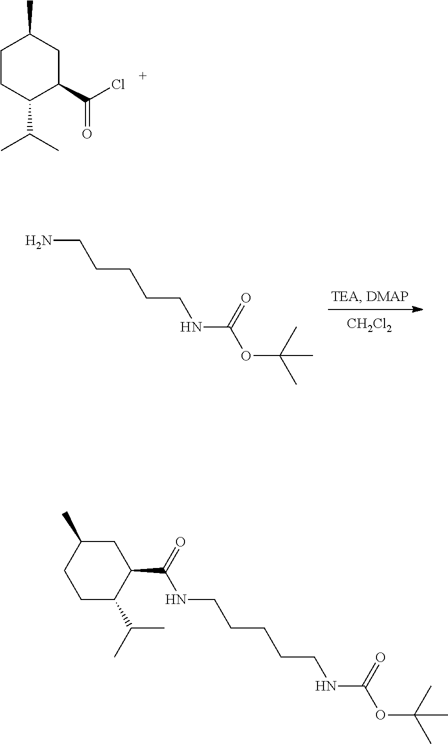

Step 2.

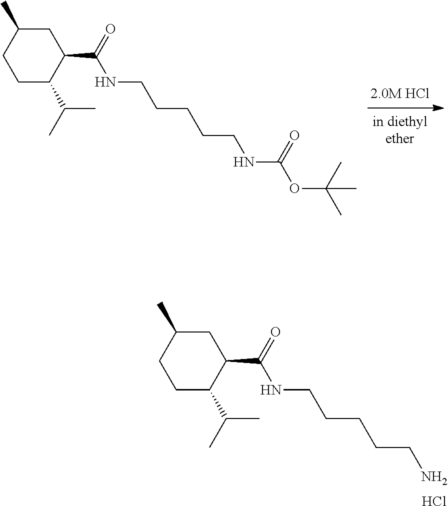 [SMILES: CC(C)[C@@H]1CC[C@@H](C)C[C@H]1C(=O)NCCCCCNC(=O)OC(C)(C)C ; "in diethylether" ; CC(C)[C@@H]1CC[C@@H](C)C[C@H]1C(=O)NCCCCCN ; Cl]

Step 1. To a 250 mL, round-bottom flask (Flask A) was added 1.0 gram (4.9 mmols) of the starting tert-butyl (5-aminopentyl)carbamate and 30 ml of anhydrous methylene chloride. The reaction flask A was immersed in an ice bath. To a separate vial B was added 2.21 grams (21.8 mmols) of triethylamine (TEA) and 10 ml of anhydrous methylene chloride. To a separate vial C was added 0.118 grams (1.0 mmols) of 4-(dimethylaminopyridine) (DMAP) and 10 mL of anhydrous methylene chloride. To a separate vial D was added 0.925 grams (4.6 mmols) of (1R,2S,5R)-2-isopropyl-5-methylcyclohexane-1-carbonyl chloride and 10 mL of anhydrous methylene chloride. The contents of vial B, then C and then D were added to the reaction flask A with magnetic mixing at 200 r.p.m. and while purging the flask headspace with dry nitrogen. The reaction flask A was closed with a rubber septum and the contents allowed to mix in the melting ice bath and under static, dry nitrogen atmosphere for 24 hours. Following the reaction period the reaction mixture was added to a 250 mL separatory funnel and extracted with three 50 ml aliquots of 1.0 N HCl, two 50 mL aliquots of 1.0 N NaOH, one 50 mL aliquot of saturated sodium bicarbonate, and one 50 mL aliquot of saturated potassium chloride. The extracted organic layer was dried over anhydrous sodium sulfate overnight. The dried solution was filtered through Whatman #4 filter, and the solvent was removed in vacuo to give 1.48 grams (4.0 mmols) of tert-butyl (5-((1R,2S,5R)-2-isopropyl-5-methylcyclohexane-1-carboxamido)pentyl)carbamate. MS(ESI) m/z 369 (MH⁺).

Step 2. To a 250 mL, round-bottom flask (Flask B) was added 1.34 grams (3.6 mmols) of the intermediate tert-butyl (5-((1R,2S ,5R)-2-isopropyl-5-methylcyclohexane-1-carboxamido)pentyl)carbamate and 100 mL of a 2.0 M hydrogen chloride solution in diethyl ether while purging the reaction headspace with dry nitrogen and magnetically mixing at 200 r.p.m. The reaction flask was closed with a rubber septum and allowed to react for 24 hours. Following the reaction period the white, precipitated solids were filtered and washed with two 20 ml aliquots of diethyl ether. The recovered solids were dissolved in 10 mL of ethanol and re-precipitated into 100 mL of diethyl ether. The solids were filtered and washed with two additional aliquots of diethyl ether. Solvent was removed from the residue in vacuo to yield the title compound (1R,2S ,5R)-N-(5-aminopentyl)-2-isopropyl-5-methylcyclohexane-1-carboxamide hydrochloride, as an off-white solid. MS(ESI) m/z 269 (MH⁺).

Using the procedure described above (steps 1 and 2) and the appropriate Boc protected diamine, the following carboxamides may be prepared by one skilled in the art:
(1R,2S ,5R)-N-(3-aminopropyl)-2-isopropyl-5-methylcyclohexane-1-carboxamide hydrochloride
(1R,2S ,5R)-N-(4-aminobutyl)-2-isopropyl-5-methylcyclohexane-1-carboxamide hydrochloride
(1R,2S ,5R)-N-(6-aminohexyl)-2-isopropyl-5-methylcyclohexane-1-carboxamide hydrochloride

Purity of isomers produced via the outlined synthetic routes:

TABLE 6 contains the percent peak areas from a chiral supercritical fluid chromatographic (SFC) separation of isomeric species from a non-stereospecific synthesis that produced isomers of molecules 2, 17, and 33, whose structures are shown on TABLE 7. In contrast, the isomeric purity measured by SFC resulting from the stereospecific synthetic routes described within is excellent, as demonstrated for molecule 17 (DSL), molecule 33 (DRL), and molecule 2 (LSL), whose structures are provided on TABLE 7.

TABLE 6

| | | % of Total Peak Area Per Sample | | | |
|---|---|---|---|---|---|
| | | Non- | DSL | DRL | LSL |
| | Retention | Stereo- | Stereo- | Stereo- | Stereo- |
| Molecule 17 | Time | specific | specific | specific | specific |
| Isomer | (min) | Synthesis | Synthesis | Synthesis | Synthesis |
| A | 34.0 | 0 | 0 | 0 | 100 |
| B | 35.6 | 2 | 0 | 0 | 0 |
| C | 37.0 | 43 | 99 | 0 | 0 |
| D | 39.8 | 43 | 0 | 99 | 0 |
| E | 41.4 | 3 | 0 | 0 | 0 |
| F | 42.4 | 2 | 0 | 0 | 0 |
| G | 47.7 | 2 | 0 | 0 | 0 |
| H | 50.5 | 1 | 0 | 0 | 0 |
| I | 55.9 | 3 | 0 | 0 | 0 |
| J | 61.1 | 1 | 0 | 0 | 0 |
| K | 66.7 | 1 | 0 | 0 | 0 |

Table 6 outlines the synthesized isomers and their relative purity, highlighting the importance of controlling the stereochemistry during synthesis. The molecules synthesized via the outlined routes had purities of 99% or more. These pure molecules were then further tested in the TRPM8 cell line for activity on the TRPM8 receptor and formulated into a dentifrice for evaluation in vivo.

### Example 52

### EC50 Analysis of Sensate Analogs Using TRPM8 Activation

It is now well established that sensations such as cool or cold can be attributed to activation of receptors at peripheral nerve fibers by a stimulus such as low temperature or a chemical coolant, which produces electrochemical signals that travel to the brain, which then interprets, organizes and integrates the incoming signals into a perception or sensation. Different classes of receptors have been implicated in sensing cold temperatures or chemical coolant stimuli at mammalian sensory nerve fibers. Among these receptors, a major candidate involved in sensing cold has been identified and designated as cold- and menthol-sensitive receptor (CMR1) or TRPM8. The TRPM8 nomenclature for the receptor comes from its characterization as a non-selective cation channel of the transient receptor potential (TRP) family, which is activated by stimuli including low temperatures, menthol and other chemical coolants. However, the precise mechanisms underlying the perception of a pleasant cooling sensation on skin or oral surfaces are presently not clearly understood. While it has been demonstrated that the TRPM8 receptor is activated by menthol and other coolants, it is not fully understood what other receptors may be involved, and to what extent these receptors need to be stimulated or perhaps suppressed in order for the overall perceived sensation to be pleasant, cooling and refreshing. For example, menthol is widely used as a cooling agent, but menthol can also produce other sensations including tingling, burning, prickling and stinging as well as a minty smell and bitter taste. Thus, it can be inferred that menthol acts on many different receptors, including cold, warm, pain and taste receptors.

The cooling receptor conventionally known as TRPM8 or the menthol receptor has been demonstrated as a means to differentiate intensity and duration of organic molecules that initiate and propagate the non-thermal cooling perception (D. D. Mckemy, The Open Drug Discovery Journal 2:81-88 2010). McKemy reported the EC50 values of many agonists to TRPM8 which span the range of 100 nM to 19 mM, thus showing the channel can be activated across a wide range of structures at varying concentrations. This channel also has the nomenclature of CRM1 and TRPP8. The later was designated as such due to its identification with prostate cells, where it was employed as a means to identify molecules targeted towards prostate cancer.

The term “TRPM8” or “TRPM8 receptor”, as used herein, refers to cold- and menthol-sensitive receptor (CMR1) or TRPM8. The TRPM8 nomenclature for the receptor comes from its characterization as a non-selective cation channel of the transient receptor potential (TRP) family that is activated by stimuli including low temperatures, menthol and other chemical coolants. The TRPM8 receptor is provided as SEQ ID NO: 1.

The term “TRPM8 agonist”, as used herein, refers to any compound, which when added to a TRPM8 receptor, according to the FLIPR method, as discussed herein, produces any increase in fluorescence over background.

| SEQ ID NO | Sequence |
|---|---|
| 1 | Human TRPM8 DNA sequence |

A sequence listing that sets forth the nucleotide sequences for SEQ ID NO: 1 herein is being filed concurrently with the present application as an ASCII text file titled “14076M_Nucleotide_Sequence_Listing_ST25.” The ASCII text file was created on 18 Oct. 2016 and is 5 Kbytes in size. In accordance with MPEP § 605.08 and 37 CFR § 1.52(e), the subject matter in the ASCII text file is incorporated herein by reference.

To determine what effect, if any, test compounds (shown in TABLE 7) had on TRPM8 (SEQ ID NO: 1), activation the protocol listed below was used.

TRPM8 Protocol-FLIPR Assay

To determine whether TRPM8 is activated, the intracellular calcium ion (Ca²⁺) level was measured from transfected cells with the TRPM8 receptor sequence (SEQ ID NO:). HEK-293 (human embryonic kidney) cells stably transfected with human TRPM8 were grown in 15 ml growth medium (high glucose DMEM (Dulbecco's Modification of Eagle's Medium) supplemented with 10% FBS (fetal bovine serum), 100 ug/ml penicillin/streptomycin, 5 μg/ml blasticindin, and 100 μg/ml zeocin) in a 75 cm² flask for 3 days at 37° C. in a mammalian cell culture incubator set at 5% CO₂ Cells were detached with addition of 2 ml of trypsin-EDTA buffer (GIBCO® 25200, Invitrogen, Grand Island, N.Y.) for about 2-3 min. Trypsin was inactivated by addition of 8 ml growth medium. Cells were transferred to a 50 ml tube and centrifuged at 850 rpm for 3 minutes to remove medium. After centrifugation, a pellet of cells was formed in the bottom of the tube separating them from the supernatant solution. The supernatant was discarded and the cell pellet was suspended in 1 ml of fresh growth medium to which 5 μl (12.5 μg) of Fluo-4 AM (Molecular Probes, Inc., Eugene, Oreg.) calcium indicator was added and incubated for 30 min with gentle shaking. Fluo-4 AM is a fluorescent dye used for quantifying cellular Ca²⁺ concentrations in the 100 nM to 1 microM range. At the end of 30 minutes, 45 ml of assay buffer (1× HBSS (Hank's Balanced Salt Solution), 20 mM HEPES (4-(2-Hydroxyethyl)-1-piperazineethanesulfonic acid)) was added to wash cells and the resulting mixture was then centrifuged at 850 rpm for 3 minutes to remove excess buffer and Fluo-4 AM calcium indicator.

The pelleted cells were re-suspended in 10 ml assay buffer and 90 μl aliquots (50,000 cells) per well delivered to a 96-well assay plate containing 10 μl of test compounds (1 mM in assay buffer, final concentration 100 μM) or buffer control and incubated at room temperature for 30 minutes. After 30 minutes, a plate was placed into a fluorometric imaging plate reader (FLIPR384 from Molecular Devices, Sunnyvale, Calif.) and basal fluorescence recorded (excitation wave length 488 nm and emission wave length 510 nm). Then 20 μl of 100 mM of TRPM8 agonist WS5 coolant in the assay buffer was added and fluorescence recorded. For determining the direct effect of test compounds on TRPM8, fluorescence was measured immediately after addition of each compound (TABLE 7). Additional discussion of the FLIPR method can be found in Smart et al., Characterization using FLIPR of human vanilloid VR1 receptor pharmacology, European Journal of Pharmacology 417, 51-58 (2001) and Liu et al., Development and validation of a platelet calcium flux assay using a fluorescent imaging plate reader, Analytical Biochemistry 357, 216-224 (2006).

TABLE 7

| | | | Stereo |
|---|---|---|---|
| # | Molecule | TRPM8 | configuration |
| 1 | 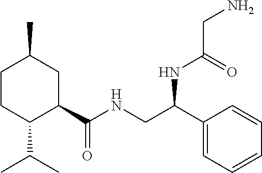 [SMILES: CC(C)[C@@H]1CC[C@@H](C)C[C@H]1C(=O)NC[C@@H](NC(=O)CN)c1ccccc1] | <0.002 | . . . SL |
| 2 | 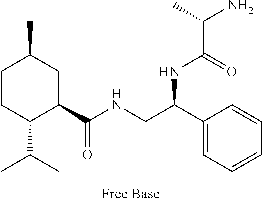 [SMILES: CC(C)[C@@H]1CC[C@@H](C)C[C@H]1C(=O)NC[C@@H](NC(=O)[C@H](C)N)c1ccccc1] | 0.0027 micro Molar | LSL |
| 3 | 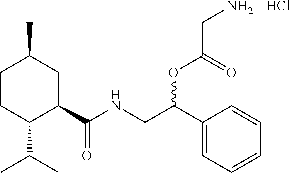 [SMILES: CC(C)[C@@H]1CC[C@@H](C)C[C@H]1C(=O)NCC(OC(=O)CN)c1ccccc1 ; Cl] | 0.00503 micro Molar | . . . R/SL |
| 4 | 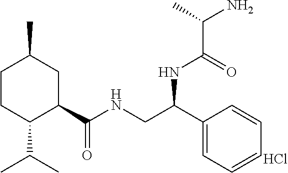 [SMILES: CC(C)[C@@H]1CC[C@@H](C)C[C@H]1C(=O)NC[C@@H](NC(=O)[C@H](C)N)c1ccccc1 ; Cl] | 0.0053 micro Molar | LSL |
| 5 | 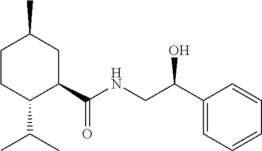 [SMILES: CC(C)[C@@H]1CC[C@@H](C)C[C@H]1C(=O)NC[C@@H](O)c1ccccc1] | 0.00674 micro Molar | . . . SL |
| 6 | 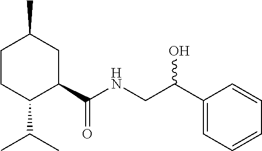 [SMILES: CC(C)[C@@H]1CC[C@@H](C)C[C@H]1C(=O)NCC(O)c1ccccc1] | 0.0072 micro Molar | . . . R/SL |
| 7 | 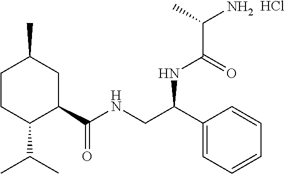 [SMILES: Cl ; CC(C)[C@@H]1CC[C@@H](C)C[C@H]1C(=O)NC[C@@H](NC(=O)[C@H](C)N)c1ccccc1] | 0.009 micro Molar | LSL |
| 8 | 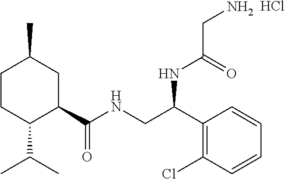 [SMILES: Cl ; CC(C)[C@@H]1CC[C@@H](C)C[C@H]1C(=O)NC[C@@H](NC(=O)CN)c1ccccc1Cl] | 0.02 micro Molar | . . . SL |
| 9 | 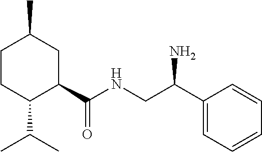 [SMILES: CC(C)[C@@H]1CC[C@@H](C)C[C@H]1C(=O)NC[C@@H](N)c1ccccc1] | 0.021 micro Molar | . . . SL |
| 10 | 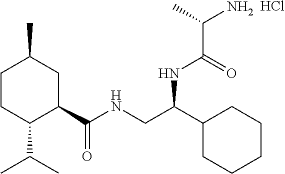 [SMILES: Cl ; CC(C)[C@@H]1CC[C@@H](C)C[C@H]1C(=O)NC[C@@H](NC(=O)[C@H](C)N)C1CCCCC1] | 0.02833 micro Molar | LSL |
| 11 | 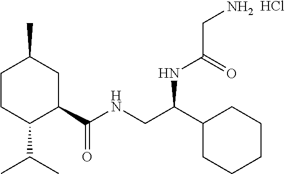 [SMILES: Cl ; CC(C)[C@@H]1CC[C@@H](C)C[C@H]1C(=O)NC[C@@H](NC(=O)CN)C1CCCCC1] | 0.03209 micro Molar | . . . SL |
| 12 | 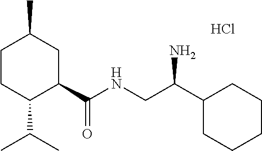 [SMILES: CC(C)[C@@H]1CC[C@@H](C)C[C@H]1C(=O)NC[C@@H](N)C1CCCCC1 ; Cl] | 0.0471 micro Molar | . . . SL |
| 13 | 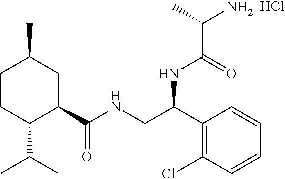 [SMILES: Cl ; CC(C)[C@@H]1CC[C@@H](C)C[C@H]1C(=O)NC[C@@H](NC(=O)[C@H](C)N)c1ccccc1Cl] | 0.052 micro Molar | LSL |
| 14 | 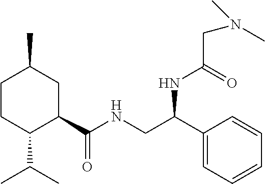 [SMILES: CC(C)[C@@H]1CC[C@@H](C)C[C@H]1C(=O)NC[C@@H](NC(=O)CN(C)C)c1ccccc1] | 0.053 micro Molar | . . . SL |
| 15 | 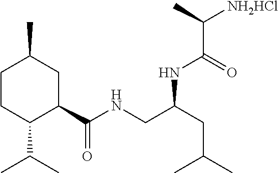 | 0.05362 micro Molar | DSL |
| 16 | 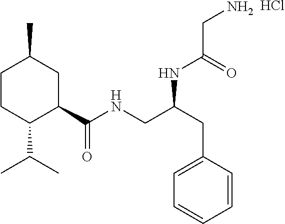 [SMILES: Cl ; CC(C)[C@@H]1CC[C@@H](C)C[C@H]1C(=O)NC[C@H](Cc1ccccc1)NC(=O)CN] | 0.058 micro Molar | . . . SL |
| 17 | 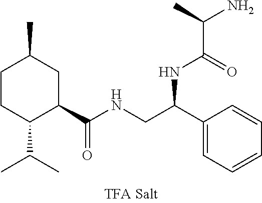 [SMILES: CC(C)[C@@H]1CC[C@@H](C)C[C@H]1C(=O)NC[C@@H](NC(=O)[C@@H](C)N)c1ccccc1] | 0.0663 micro Molar | DSL |
| 18 | 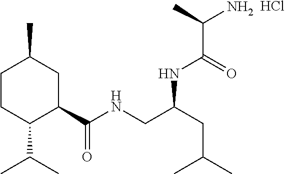 [SMILES: Cl ; CC(C)C[C@@H](CNC(=O)[C@@H]1C[C@H](C)CC[C@H]1C(C)C)NC(=O)[C@@H](C)N] | 0.0806 micro Molar | DSL |
| 19 | 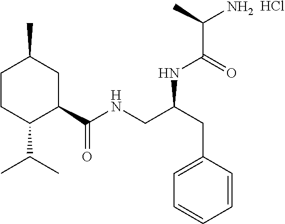 [SMILES: Cl ; CC(C)[C@@H]1CC[C@@H](C)C[C@H]1C(=O)NC[C@H](Cc1ccccc1)NC(=O)[C@@H](C)N] | 0.092 micro Molar | DSL |
| 20 | 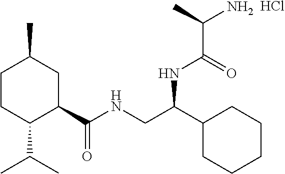 [SMILES: Cl ; CC(C)[C@@H]1CC[C@@H](C)C[C@H]1C(=O)NC[C@@H](NC(=O)[C@@H](C)N)C1CCCCC1] | 0.09519 micro Molar | DSL |
| 21 | 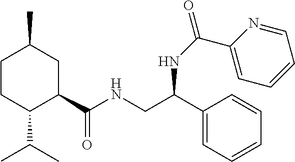 [SMILES: CC(C)[C@@H]1CC[C@@H](C)C[C@H]1C(=O)NC[C@@H](NC(=O)c1ccccn1)c1ccccc1] | 0.192 micro Molar | . . . SL |
| 22 | 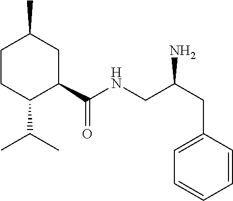 [SMILES: CC(C)[C@@H]1CC[C@@H](C)C[C@H]1C(=O)NC[C@@H](N)Cc1ccccc1] | 0.1977 micro Molar | . . . SL |
| 23 | 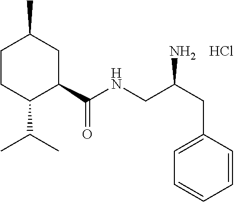 [SMILES: CC(C)[C@@H]1CC[C@@H](C)C[C@H]1C(=O)NC[C@@H](N)Cc1ccccc1 ; Cl] | 0.2598 micro Molar | . . . SL |
| 24 | 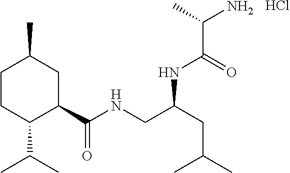 [SMILES: Cl ; CC(C)C[C@@H](CNC(=O)[C@@H]1C[C@H](C)CC[C@H]1C(C)C)NC(=O)[C@H](C)N] | 0.266 micro Molar | LSL |
| 25 | 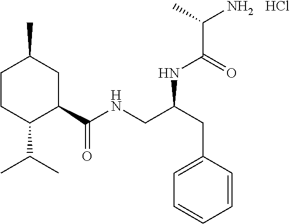 [SMILES: Cl ; CC(C)[C@@H]1CC[C@@H](C)C[C@H]1C(=O)NC[C@H](Cc1ccccc1)NC(=O)[C@H](C)N] | 0.3 micro Molar | LSL |
| 26 | 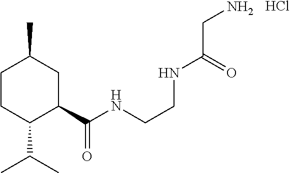 [SMILES: Cl ; CC(C)[C@@H]1CC[C@@H](C)C[C@H]1C(=O)NCCNC(=O)CN] | 0.3 micro Molar | . . . / . . . /L |
| 27 | 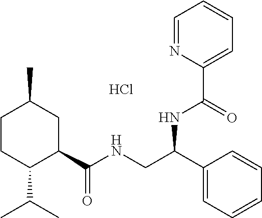 [SMILES: CC(C)[C@@H]1CC[C@@H](C)C[C@H]1C(=O)NC[C@@H](NC(=O)c1ccccn1)c1ccccc1 ; Cl] | 0.31 micro Molar | . . . SL |
| 28 | 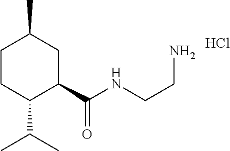 [SMILES: CC(C)[C@@H]1CC[C@@H](C)C[C@H]1C(=O)NCCN ; Cl] | 0.35 micro Molar | . . . / . . . /L |
| 29 | 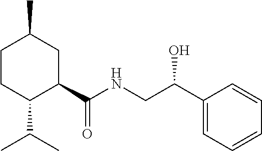 [SMILES: CC(C)[C@@H]1CC[C@@H](C)C[C@H]1C(=O)NC[C@H](O)c1ccccc1] | 0.3737 micro Molar | . . .RL |
| 30 | 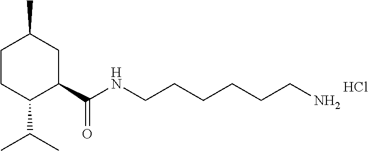 [SMILES: CC(C)[C@@H]1CC[C@@H](C)C[C@H]1C(=O)NCCCCCCN ; Cl] | 0.4055 micro Molar | . . . / . . . /L |
| 31 | 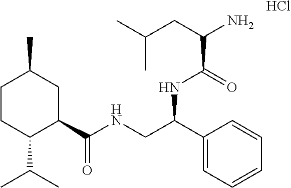 | 0.426 micro Molar | DSL |
| 32 | 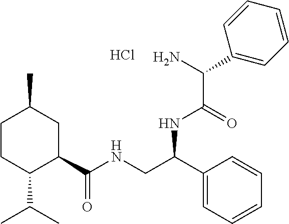 [SMILES: CC(C)[C@@H]1CC[C@@H](C)C[C@H]1C(=O)NC[C@@H](NC(=O)[C@H](N)c1ccccc1)c1ccccc1 ; Cl] | 0.46 micro Molar | LSL |
| 33 | 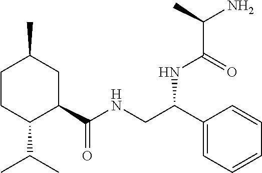 [SMILES: CC(C)[C@@H]1CC[C@@H](C)C[C@H]1C(=O)NC[C@H](NC(=O)[C@@H](C)N)c1ccccc1] | 0.4859 micro Molar | DRL |
| 34 | 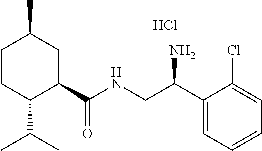 [SMILES: CC(C)[C@@H]1CC[C@@H](C)C[C@H]1C(=O)NC[C@@H](N)c1ccccc1Cl ; Cl] | 0.77 micro Molar | . . . SL |
| 35 | 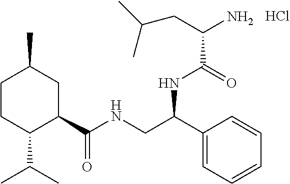 [SMILES: CC(C)C[C@H](N)C(=O)N[C@H](CNC(=O)[C@@H]1C[C@H](C)CC[C@H]1C(C)C)c1ccccc1 ; Cl] | 0.9215 micro Molar | LSL |
| 36 | 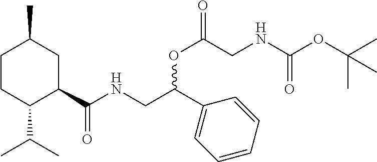 [SMILES: CC(C)[C@@H]1CC[C@@H](C)C[C@H]1C(=O)NCC(OC(=O)CNC(=O)OC(C)(C)C)c1ccccc1] | 0.99 micro Molar | . . . R/SL |
| 37 | 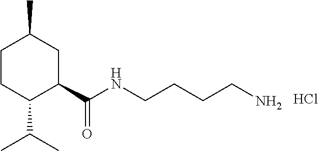 [SMILES: CC(C)[C@@H]1CC[C@@H](C)C[C@H]1C(=O)NCCCCN ; Cl] | 1.095 micro Molar | . . . / . . . /L |
| 38 | 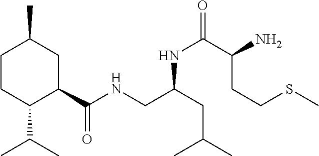 [SMILES: CSCC[C@H](N)C(=O)N[C@H](CNC(=O)[C@@H]1C[C@H](C)CC[C@H]1C(C)C)CC(C)C] | 1.1 micro Molar | DSL |
| 39 | 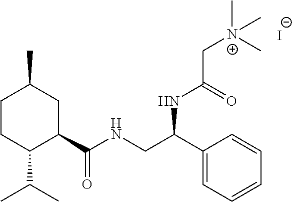 [SMILES: CC(C)[C@@H]1CC[C@@H](C)C[C@H]1C(=O)NC[C@@H](NC(=O)C[N+](C)(C)C)c1ccccc1 ; [I-]] | 1.24 micro Molar | . . . SL |
| 40 | 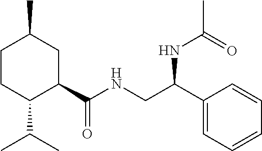 [SMILES: CC(=O)N[C@H](CNC(=O)[C@@H]1C[C@H](C)CC[C@H]1C(C)C)c1ccccc1] | 1.694 micro Molar | . . . SL |
| 41 | 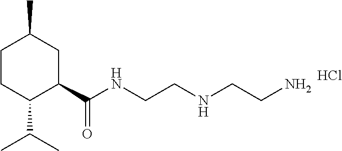 [SMILES: CC(C)[C@@H]1CC[C@@H](C)C[C@H]1C(=O)NCCNCCN ; Cl] | 2.3 micro Molar | . . . / . . . L |
| 42 | 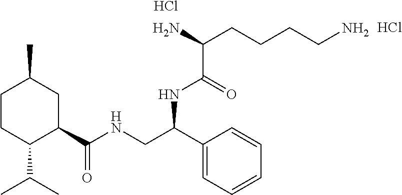 [SMILES: Cl ; Cl ; CC(C)[C@@H]1CC[C@@H](C)C[C@H]1C(=O)NC[C@@H](NC(=O)[C@@H](N)CCCCN)c1ccccc1] | 2.8 micro Molar | |
| 43 | 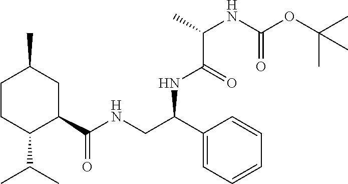 [SMILES: CC(C)[C@@H]1CC[C@@H](C)C[C@H]1C(=O)NC[C@@H](NC(=O)[C@H](C)NC(=O)OC(C)(C)C)c1ccccc1] | 3.0 | LSL |
| 44 | 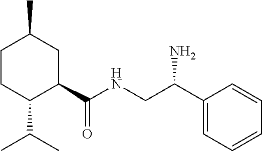 [SMILES: CC(C)[C@@H]1CC[C@@H](C)C[C@H]1C(=O)NC[C@H](N)c1ccccc1] | 3.434 micro Molar | . . .RL |
| 45 | 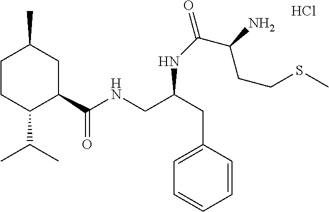 [SMILES: CSCC[C@H](N)C(=O)N[C@H](CNC(=O)[C@@H]1C[C@H](C)CC[C@H]1C(C)C)Cc1ccccc1 ; Cl] | 3.52 micro Molar | DSL |
| 46 | 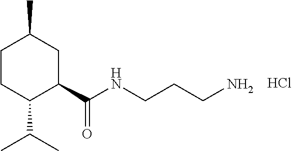 [SMILES: CC(C)[C@@H]1CC[C@@H](C)C[C@H]1C(=O)NCCCN ; Cl] | 4.338 micro Molar | . . . / . . . /L |
| 47 | 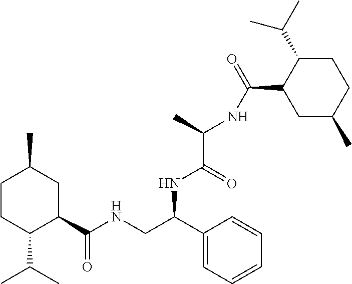 | 11.69 micro Molar | LDSL |
| 48 | 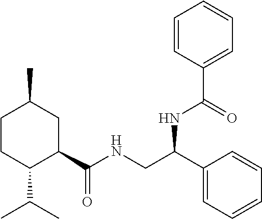 [SMILES: CC(C)[C@@H]1CC[C@@H](C)C[C@H]1C(=O)NC[C@@H](NC(=O)c1ccccc1)c1ccccc1] | 21.46 micro Molar | . . . SL |
| 49 | 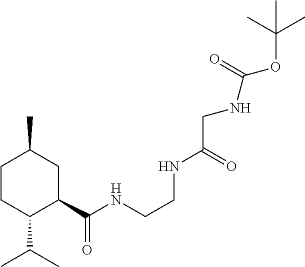 [SMILES: CC(C)[C@@H]1CC[C@@H](C)C[C@H]1C(=O)NCCNC(=O)CNC(=O)OC(C)(C)C] | 40 micro Molar | . . . / . . . /L |
| 50 | 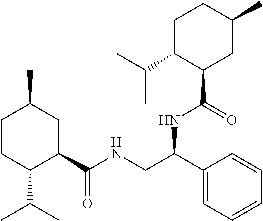 [SMILES: CC(C)[C@@H]1CC[C@@H](C)C[C@H]1C(=O)NC[C@@H](NC(=O)[C@@H]1C[C@H](C)CC[C@H]1C(C)C)c1ccccc1] | 50 micro Molar | LSL |
| 51 | 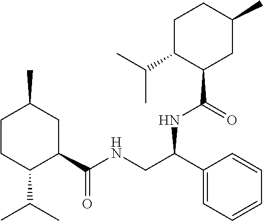 [SMILES: CC(C)[C@@H]1CC[C@@H](C)C[C@H]1C(=O)NC[C@@H](NC(=O)[C@@H]1C[C@H](C)CC[C@H]1C(C)C)c1ccccc1] | 50 micro Molar | LSL |
| 52 | 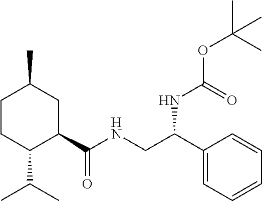 [SMILES: CC(C)[C@@H]1CC[C@@H](C)C[C@H]1C(=O)NC[C@H](NC(=O)OC(C)(C)C)c1ccccc1] | Too high to measure | . . . RL |
| 53 | 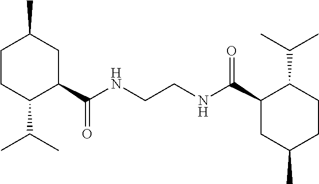 [SMILES: CC(C)[C@@H]1CC[C@@H](C)C[C@H]1C(=O)NCCNC(=O)[C@@H]1C[C@H](C)CC[C@H]1C(C)C] | Too high to measure | L . . . L |
| 54 | 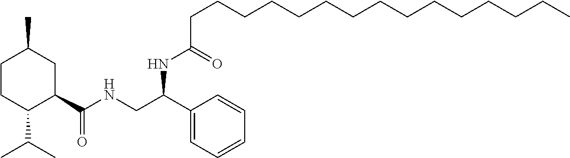 [SMILES: CCCCCCCCCCCCCCCC(=O)N[C@H](CNC(=O)[C@@H]1C[C@H](C)CC[C@H]1C(C)C)c1ccccc1] | Too high to measure | . . . SL |
| 55 | 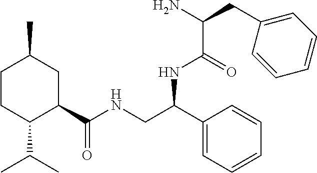 [SMILES: CC(C)[C@@H]1CC[C@@H](C)C[C@H]1C(=O)NC[C@@H](NC(=O)[C@@H](N)Cc1ccccc1)c1ccccc1] | Too high to measure | DSL |
| 56 | 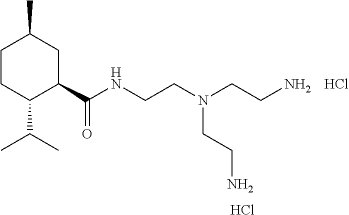 [SMILES: CC(C)[C@@H]1CC[C@@H](C)C[C@H]1C(=O)NCCN(CCN)CCN ; Cl ; Cl] | Too high to measure | . . . / . . . /L |
| 57 | 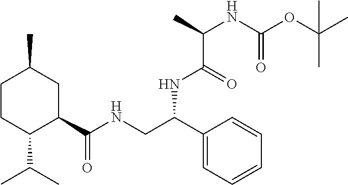 [SMILES: CC(C)[C@@H]1CC[C@@H](C)C[C@H]1C(=O)NC[C@H](NC(=O)[C@@H](C)NC(=O)OC(C)(C)C)c1ccccc1] | Too high to measure | DRL |
| 58 | 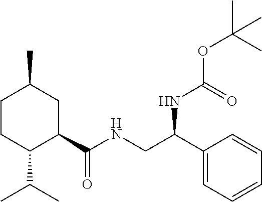 [SMILES: CC(C)[C@@H]1CC[C@@H](C)C[C@H]1C(=O)NC[C@@H](NC(=O)OC(C)(C)C)c1ccccc1] | Too high to measure | . . . SL |
| 59 | 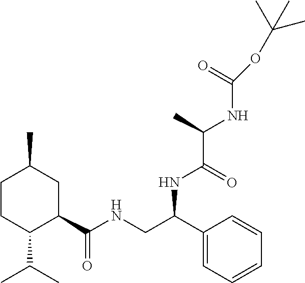 [SMILES: CC(C)[C@@H]1CC[C@@H](C)C[C@H]1C(=O)NC[C@@H](NC(=O)[C@@H](C)NC(=O)OC(C)(C)C)c1ccccc1] | Too high to measure | DSL |
| 60 | 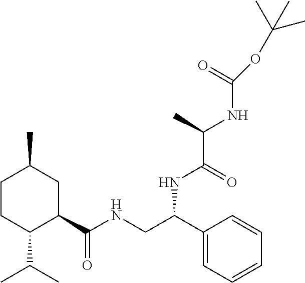 [SMILES: CC(C)[C@@H]1CC[C@@H](C)C[C@H]1C(=O)NC[C@H](NC(=O)[C@@H](C)NC(=O)OC(C)(C)C)c1ccccc1] | Too high to measure | DRL |
| 61 | 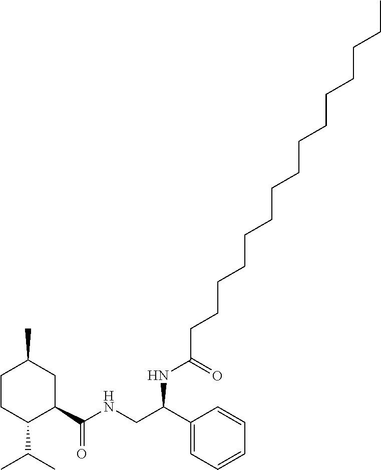 [SMILES: CCCCCCCCCCCCCCCC(=O)N[C@H](CNC(=O)[C@@H]1C[C@H](C)CC[C@H]1C(C)C)c1ccccc1] | Too high to measure | . . . SL |

The EC50 values shown in TABLE 7 provided examples of the unique sensates that were synthesized via the economical synthetic route outlined in previous examples.

### Example 53

### Cooling Dentifrice Formulation

Dentifrices were prepared using conventional methods, such as the protocols described in U.S. Pat. No. 8,747,814, which contained no coolant (SAMPLE A) or having a coolant from TABLE 7 (SAMPLES B and C), in a flavor (peppermint) at 10 parts per million (ppm).

TABLE 8
Dentifrice formulations containing the compounds from TABLE 7

| | Samples | | |
|---|---|---|---|
| | A | | |
| Ingredient | (Control) | B | C |
| FD&C Blue #1 Color Solution | 0.045% | 0.045% | 0.045% |
| Sodium Fluoride | 0.243% | 0.243% | 0.243% |
| CARBOMER 956 | 0.300% | 0.300% | 0.300% |
| Sodium Saccharin | 0.300% | 0.300% | 0.300% |
| Sodium Phosphate, Monobasic, | 0.419% | 0.419% | 0.419% |
| Monohydrate | | | |
| Titanium Dioxide | 0.525% | 0.525% | 0.525% |
| Carboxymethycellulose Sodium | 0.800% | 0.800% | 0.800% |
| Wintergreen Flavor | 1.000% | 1.000% | 1.000% |
| Coolant | 0% | — | — |
| Coolant Molecule #17 (TABLE 7) | — | 0.001% | — |
| Coolant Molecule #33 (TABLE 7) | — | — | 0.001% |
| Tribasic Sodium Phosphate Dodeca- | 1.100% | 1.100% | 1.100% |
| hydrate | | | |
| Sodium Lauryl Sulfate 28% Solution | 4.000% | 4.000% | 4.000% |
| Silica, Dental Type, NF (Zeodent | 15.000% | 15.000% | 15.000% |
| 119) | | | |
| SORBITOL SOLUTION LRS USP | 54.673% | 54.673% | 54.673% |
| Water Purified, USP, PhEur, JP, JSCI | QS* | QS* | QS* |

*QS refers to the term quantum sufficit, meaning as much as suffices, where the remainder of the formula hole is filled with this substance.

Sensory evaluation studies of coolant activity were conducted using a methodology patterned after the techniques described in M.C. Meilgaard, et al., Sensory Evaluation Techniques, 4th Ed. (2007). Five panelists brushed with a dentifrice from TABLE 8, having no coolant (SAMPLE A) or having a coolant from TABLE 7 (SAMPLES B and C), for two minutes, in a flavor (peppermint) at 10 parts per million (ppm). After brush expectoration, panelists then rinsed their mouth with 15 ml of an aqueous rinse and expectorated. As shown in TABLE 9, panelists then evaluated cooling intensity, assigning a number between 0, which is no cooling sensation, to 90, which is a sensation as cold as ice.

TABLE 9
Panelists evaluated cooling properties (n = 3)

| | | Time | | | | | | |
|---|---|---|---|---|---|---|---|---|
| | | Initial 0 | | 1 | 2 | 3 | 6 | 12 |
| SAMPLE | | minutes | 30 min. | hour | hour | hour | hour | hour |
| Dentifrice A | Sensory | Low | None | None | None | None | None | None |
| (Control) | measures - | | | | | | | |
| Dentifrice B: | None, | Medium | High | High | High | High | High | High |
| D(S)L (Molecule | Low, | | | | | | | |
| #17 Table 7) | Medium, | | | | | | | |
| Dentifrice C: | High | Medium | Low | None | None | None | None | None |
| D(R)L (Molecule | | | | | | | | |
| #33 Table 7) | | | | | | | | |

As noted from TABLE 9, the D(S)L isomer from the reaction product outlined in EXAMPLE 1 exhibited high cooling over the course of 12 hours after brushing. Whereas, the D(R)L isomer trailed off after 30 minutes. This data indicated that the D(S)L isomer contributed to the cooling response noted by the panelists.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm ”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for preparing an N-menthanecarboxamide derivative of the following formula (I) comprising: 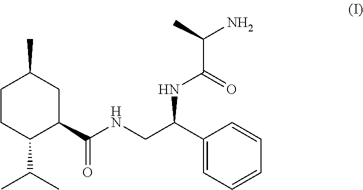 [SMILES: CC(C)[C@@H]1CC[C@@H](C)C[C@H]1C(=O)NC[C@@H](NC(=O)[C@@H](C)N)c1ccccc1]
and pharmaceutically acceptable salts thereof;
A) a coupling reaction between an activated derivative of the p-menthane-3-carboxylic acid of Formula (II): 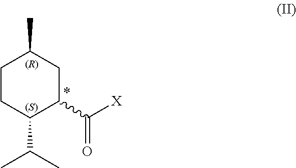
wherein X=a suitable leaving group; and a primary amine of the general formula (III): 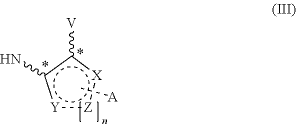
to produce a compound of the general formula (IV):

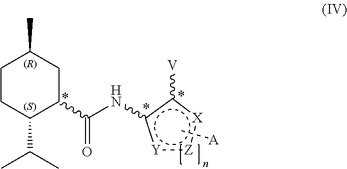

2. A method for preparing an N-menthanecarboxamide derivative of the following formula (V) comprising: 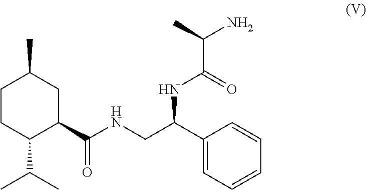 [SMILES: CC(C)[C@@H]1CC[C@@H](C)C[C@H]1C(=O)NC[C@@H](NC(=O)[C@@H](C)N)c1ccccc1]
and pharmaceutically acceptable salts thereof;
A) a coupling reaction between an activated derivative of the p-menthane-3-carboxylic acid of Formula (VI): 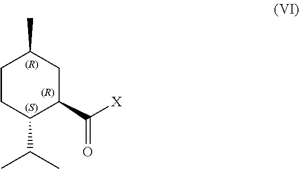
wherein X=a suitable leaving group; and a primary amine of the general formula (VII): 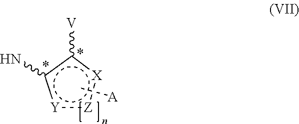
to produce a compound of the general formula (VIII): [chemical structure]
